(19) United States
(12) Patent Application Publication
(10) Pub. No.: US 2021/0220357 A1
(43) Pub. Date: Jul. 22, 2021

(54) 1,3-THIAZOL-2-YL SUBSTITUTED BENZAMIDES FOR THE TREATMENT OF DISEASES ASSOCIATED WITH NERVE FIBER SENSITIZATION

(71) Applicants: Bayer Aktiengesellschaft, Leverkusen (DE); Bayer Pharma Aktiengesellschaft, Berlin (DE)

(72) Inventors: Lucas Hudson HOFMEISTER, Wuppertal (DE); Oliver Martin FISCHER, Berlin (DE)

(73) Assignees: Bayer Aktiengesellschaft, Leverkusen (DE); Bayer Pharma Aktiengesellschaft, Berlin (DE)

(21) Appl. No.: 17/055,482

(22) PCT Filed: May 14, 2019
(86) PCT No.: PCT/EP2019/062329

§ 371 Date: Nov. 13, 2020

(30) Foreign Application Priority Data
May 15, 2018 (EP) 18172405.5

(51) Int. Cl. A61K 31/506; A61K 31/5377; A61P 11/00; A61P 9/12; A61K 9/00

(52) U.S. Cl. CPC A61K 31/506; A61K 31/5377; A61K 9/0053; A61P 9/12; A61P 11/00

(57) ABSTRACT

The present invention relates to use 1,3-thiazol-2-yl substituted benzamide compounds of general formula (I) as described and defined herein, to pharmaceutical compositions and combinations comprising said compounds for the treatment or prophylaxis of diseases which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

The present invention relates to the use of 1,3-thiazol-2-yl substituted benzamide compounds of general formula (I) as a sole agent or in combination with other active ingredients as well as to the use of pharmaceutical compositions and combinations comprising said compounds for the treatment or prophylaxis of diseases which are associated with nerve fiber sensitization and/or autonomic imbalance, in particular cardiovascular diseases, heart failure and hypertension.

### BACKGROUND OF THE INVENTION

The present invention relates to the use of chemical compounds that inhibit P2X3 receptor for the treatment of diseases associated with nerve fiber sensitization P2X purinoceptor 3 is a protein that in humans is encoded by the P2RX3 gene (Garcia-Guzman M, Stuehmer W, Soto F, 1997, Brain Res Mol Brain Res 47 (1-2): 59-66). The product of this gene belongs to the family of purinoceptors for ATP. This receptor functions as a ligand-gated ion channel and transduces ATP-evoked nociceptor activation.

P2X purinoreceptors are a family of ligand-gated ion channels that are activated by ATP. To date, seven members of this family have been cloned, comprising P2X1-7 (Burnstock 2013, front Cell Neurosci 7:227). These channels can exist as homomers and heteromers (Saul 2013, front Cell Neurosci 7:250). Purines, such as ATP, have been recognized as important neurotransmitters and by acting via their respective receptors they have been implicated in various physiological and pathophysiological roles (Burnstock 1993, Drug Dev Res 28:196-206; Burnstock 2011, Prog Neurobiol 95:229-274; Jiang 2012, Cell Health Cytoskeleton 4:83-101).

Among the P2X family members, in particular the P2X3 receptor has been recognized as an important mediator of nociception (Burnstock 2013, Eur J Pharmacol 716:24-40; North 2003, J Phyiol 554:301-308; Chizh 2000, Pharmacol Rev 53:553-568). It is mainly expressed in dorsal root ganglia in a subset of nociceptive sensory neurons. During inflammation the expression of the P2X3 receptor is increased, and activation of P2X3 receptor has been described to sensitize peripheral nerves (Fabretti 2013, front Cell Neurosci 7:236).

The prominent role of the P2X3 receptor in nociception has been described in various animal models, including mouse and rat models for acute, chronic and inflammatory pain. P2X3 receptor knock-out mice show a reduced pain response (Cockayne 2000, Nature 407:1011-1015; Souslova 2000, Nature 407:1015-1017). P2X3 receptor antagonists have been shown to act anti-nociceptive in different models of pain and inflammatory pain (Ford 2012, Purin Signal 8 (Suppl 1):S3-S26). The P2X3 receptor has also been shown to integrate different nociceptive stimuli. Hyperalgesia induced by PGE2, ET-1 and dopamine have all been shown to be mediated via release of ATP and activation of the P2X3 receptor (Prado 2013, Neuropharm 67:252-258; Joseph 2013, Neurosci 232C: 83-89).

Besides its prominent role in nociception and in pain-related diseases involving both chronic and acute pain, the P2X3 receptor has been shown to be involved in genitourinary, gastrointestinal, cardiovascular and respiratory conditions and disorders, including overactive bladder, chronic cough, heart failure and hypertension (Ford 2013, front Cell Neurosci 7:267; Burnstock 2014, Purin Signal 10(1):3-50; Pijacka et al, Nat Med. 2016. 22(10): 1151-1159). In these examples, ATP release is involved in activation of reflex pathways including contraction of bladder and lung muscles, and the peripheral chemoreflex.

P2X3 subunits do not only form homotrimers but also heterotrimers with P2X2 subunits. P2X3 subunits and P2X2 subunits are also expressed on nerve fibers innervating the tongue, therein taste buds (Kinnamon 2013, front Cell Neurosci 7:264). In a physiological setting, receptors containing P2X3 and/or P2X2 subunits are involved in the transmission of taste from the tongue (bitter, sweet, salty, umami and sour). Recent data show that while blocking the P2X3 homomeric receptor alone is important to achieve anti-nociceptive efficacy, non-selective blockade of both the P2X3 homomeric receptor and the P2X2/3 heteromeric receptor leads to changes in taste perception which might limit the therapeutic use of non-selective P2X3 and P2X2/3 receptor antagonists (Ford 2014, purines 2014, abstract book p15). Therefore, compounds that differentiate between P2X3 and P2X2/3 receptors are highly desirable.

Compounds blocking both the exclusively P2X3 subunit containing ion channel (P2X3 homomer) as well as the ion channel composed of P2X2 and P2X3 subunit (P2X2/3 heterotrimer) are called P2X3 and P2X2/3 nonselective receptor antagonists (Ford, Pain Manag 2012, 2(3), 267-77). Clinical Phase II trials demonstrated that AF-219, a P2X3 antagonist, leads to taste disturbances in treated subjects by affecting taste sensation via the tongue (e.g. Abdulqawi et al, Lancet 2015, 385 (9974), 1198-1205; Strand et al, 2015 ACR/ARMP Annual Meeting, Abstract 2240). This side effect has been attributed to the blockade of P2X2/3 channels, i.e. the heterotrimer (A. Ford, London 2015 Pain Therapeutics Conference, congress report). Both P2X2 and P2X3 subunits are expressed on sensory nerve fibers innervating the tongue. Knock-out animals deficient for P2X2 and P2X3 subunits show reduced taste sensation and even taste loss (Finger et al, Science 2005, 310 (5753), 1495-99), whereas P2X3 subunit single knock-outs exhibit a mild or no change in phenotype with respect to taste. Moreover, two distinct populations of neurons have been described in the geniculate ganglion expressing either P2X2 and P2X3 subunits or P2X3 subunit alone (Vandenbeuch et al, J Physiol, 2015, 593(Pt 5): 1113-1125). The population expressing P2X2/P2X3 heterotrimers has been described as being less sensitive to a non-selective P2X2/P2X3 antagonist compared to the population expressing P2X3 homomers, i.e. requiring a higher concentration of this antagonist to be inhibited. In an in vivo setting assessing taste preference towards an artificial sweetener via a lickometer, only at very high free plasma levels (>100 μM) effects on taste were observed, indicating that the less sensitive P2X2 and P2X3 subunit expressing population plays a major role in taste sensation than the P2X3 subunit expressing population (Vandenbeuch et al, J Physiol, 2015, 593(Pt 5): 1113-1125). Hence, as a modified taste perception has profound effects on the quality of life of patients, P2X3-homomeric receptor-selective antagonists are deemed to be superior towards non-selective receptor antagonists and are considered to represent a solution towards the problem of insufficient patient compliance during chronic treatment as indicated by increased drop-out rates during Phil trials (Strand et al, 2015 ACR/ARMP Annual Meeting, Abstract 2240 and A. Ford, London 2015 Pain Therapeutics Conference, congress report).

Increased sympathetic nervous system (SNS) activity and sympathetic neural factors such as norepinephrine (NE, also known as noradrenaline) are involved in the genesis of cardiovascular disease (CVD) in general (Grassi et al, Circ Res, 2015, 116(6):976-990). Common comorbidities with heart failure (HF) and CVD are also associated with increased sympathetic tone and decreased parasympathetic tone, termed autonomic imbalance. Taken together, clinical studies indicate that patients suffering from autonomic imbalance have decreased exercise tolerance, higher incidence of central sleep apneas, higher incidence of arrhythmias, and increased mortality (Joyner, J Physiol, 2016, 549(14): 4009-4013). Autonomic imbalance is an independent predictor of mortality in HF and CVD patients regardless of the etiology of the condition and is caused by chronic pathological over-activation of afferent inputs such as peripheral chemoreceptors.

Recent preclinical and clinical studies have demonstrated that the carotid body peripheral chemoreflex should be considered as a target for cardiovascular diseases associated with autonomic imbalance (Del Rio et al, J Am Coll Cardiol, 2013, 62(25):2422-2430; McBryde et al, Nat Commun, 2013, 4:2395; Niewinsky et al, Int J Cardiol, 2013, 168(3):2506-2509; Paton et al, Hypertension, 2013, 61(1):5-13; Marcus et al, J Physiol, 2014, 592(2):391-408; Del Rio et al, Exp Physiol, 2015, 100(2):136-142). Chemoreflex hypersensitivity has been demonstrated in animal models of CVD with different etiology including: genetic modifications, chronic intermittent hypoxia, myocardial infarction, rapid ventricular pacing, genetic cardiomyopathy, and pressure overload.

Increased chemoreflex sensitivity is observed in 40-60% of optimally treated HF patients (Giannoni et al, J Am Coll Cardiol, 2009, 53(21):1975-1980; Niewinski et al, J Card Fail, 2013, 19(6):408-415). Chemoreflex hypersensitivity is also associated with a higher prevalence of unstable ventilatory control during wakefulness, ventilatory insufficiency during exercise, sleep related breathing disorders, Cheyne-Stokes respiration, persistent atrial fibrillation, and paroxysmal ventricular tachycardia, and impaired baroreflex control of blood pressure (Ponikowski et al, Circulation. 2001. 104(5):544-549; Corra et al, Circulation, 2006, 113(1):44-50; Giannoni et al, Clin Sci (Lond). 2008. 114(7):489-497; Despas et al, J Hypertens, 2012, 30(4):753-760; Dempsey and Smith, Adv Exp Med Biol. 2014. 758:343-349; Andrade et al, Biomed Res Int. 2015. 467597; Floras and Ponikowski, Eur Heart J, 2015, 36(30):1974-1982b; Grassi et al, Circ Res, 2015, 116(6):976-990).

In the case of CVD, neurotransmitter release, including ATP release from Type I and Type II glumus cells of the carotid body (glomus caroticum) is involved in the the physiological response to hypoxia. Recent studies (Pijacka et al, Nat Med, 2016, 22(10): 1151-1159) demonstrate that overexpression of P2X3 in the carotid body of spontaneously hypertensive rats increases tonic activation of the peripheral chemoreflex leading to increased sympathetic nervous system activity and autonomic imbalance (Pijacka et al, Nat Med, 2016, 22(10): 1151-1159). Therefore blockade of P2X3 could be considered as a treatment option for CVD associated with tonically active or hypersensitive peripheral chemoreflex.

WO2015/027212 (Afferent Pharmaceutical Inc.) discloses new diaminopyrimidine compounds having activity as antagonists of P2X purinergic receptors, and methods for treatment of diseases associated with P2X receptors comprising administering an effective amount of a diaminopyrimidine compound. More particularly, methods are provided for using P2X3 and/or P2X2/3 antagonists in the treatment of cough, chronic cough and urge to cough in respiratory conditions and disorders.

Afferent Pharmaceuticals is developing AF-219 (5-(2,4-diamino-pyrimidin-5-yloxy)-4-isopropyl-2-methoxy-benzenesulfonamide), which is an oral, small molecule P2X3 antagonist, for the potential treatment of chronic cough and pain, including chronic bladder pain syndrome and osteoarthritis pain, and asthma. Several clinical trials are ongoing, amongst them for example an US phase II trial in patients with idiopathic pulmonary fibrosis with persistent cough and breathlessness (ClinicalTrials.gov Identifier: NCT02502097) as well as a phase IIb cough trial in patients with refractory chronic cough (NCT02349425) which are completed.

With regard to CVD and hypertension, chemoreflex hypersensitivity persists in patients that are treated under the current standard of care (Neurohumoral blockade), including beta adrenergic antagonism, aldosterone receptor antagonism, angiotensin converting enzyme inhibition, and/or angiotensin receptor block (Ponikowski et al, Circulation, 2001, 104(5):544-549; Soares Barreto-Filho et al, Circulation, 2001, 104(15):1792-1798; Giannoni et al, Clin Sci (Lond), 2008, 114(7):489-497; Niewinski et al, Exp Physiol, 2014, 99(3):552-561; Mirizzi et al, PLoS One, 2016, 11(4):e0153510). In these studies, patients that demonstrate chemoreflex hypersensitivity have worsened outcomes compared to patients with normal chemoreflex sensitivity. Current standard of care therapies do not pharmacologically inhibit the peripheral chemoreflex. Therefore, a significant residual risk exists in these patients in spite of optimal treatment with standard of care therapies. Because P2X3 overexpression in the type I glomus cell of the carotid body is associated with chemoreflex hypersensitivity and cardiovascular disease, P2X3 inhibitor compounds could be used to attenuate chemoreflex hypersensitivity as a treatment for cardiovascular disease.

Thus, there is an urgent need for medicaments which are effective in the treatment of diseases which are associated with nerve fiber sensitization, and/or other pathological conditions associated with autonomic imbalance, for example caused by increased chemoreceptor sensitivity like cardiovascular diseases, heart failure and hypertension, which do not have the disadvantages of the prior art. These disadvantages include not directly targeting chemoreflex hyper-sensitivity and dysguesia associated with dual P2X2/3 blockade.

The underlying problem of the present invention therefore lies in the provision of medication for long-term oral treatment of diseases associated with nerve fiber sensitization and/or other pathological conditions associated with autonomic imbalance caused by increased chemoreceptor sensitivity like cardiovascular diseases, heart failure and hypertension, which are related to increased activity of P2X3 receptors.

### SUMMARY OF THE INVENTION

It has now been found, and this constitutes the basis of the present invention, that compounds of the general formula (I)

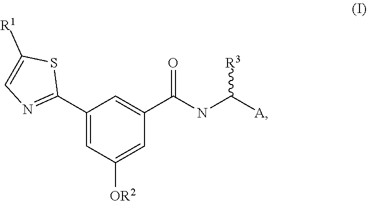

in which
R¹ represents a halogen atom, C₁-C₄-alkyl or C₃-C₆-cycloalkyl, wherein C₁-C₄-alkyl is optionally substituted with 1-5 halogen atoms which are the same or different;
R² represents —C₂-C₆-alkyl-OR⁴, —(CH₂)_{q}—(C₃-C₇-cycloalkyl), —(CH₂)_{q}-(6- to 12-membered heterobicycloalkyl), —(CH₂)_{q}-(4- to 7-membered heterocycloalkyl), —(CH₂)_{q}-(5- to 10-membered heteroaryl) or —C₂-C₆-alkynyl; and
   wherein said —(CH₂)_{q}—(C₃-C₇-cycloalkyl), —(CH₂)_{q}-(6- to 12-membered heterobicycloalkyl) and —(CH₂)_{q}-(4- to 7-membered heterocycloalkyl) are optionally substituted with one or more substituents which are the same or different, at any ring carbon atom and selected from the group consisting of
   C₁-C₄ alkyl, optionally substituted with 1-5 halogen atoms which are the same or different, a halogen atom, —NR^{a}R^{b}, COOR⁵ and oxo (═O); and
   wherein independently any ring nitrogen atom, if present in said —(CH₂)_{q}-(6- to 12-membered heterobicycloalkyl) and —(CH₂)_{q}-(4- to 7-membered heterocycloalkyl) is substituted with R^{c}; and
   wherein said —(CH₂)_{q}-(5- to 10-membered heteroaryl) is optionally substituted with one or more substituents which are the same or different, and selected from the group consisting of C₁-C₄-alkyl, optionally substituted with 1-5 halogen atoms which are the same or different, a halogen atom, —NR^{a}R^{b} and —COOR⁵;
R³ represents hydrogen or C₁-C₄-alkyl, which is optionally substituted with 1-5 halogen atoms which are the same or different;
R⁴ and R⁵ represent hydrogen or C₁-C₄-alkyl;
R^{a} and R^{b} represent hydrogen or C₁-C₄-alkyl;
R^{c} represents hydrogen, C₁-C₄-alkyl, optionally substituted with 1-5 halogen atoms which are the same or different, —C(O)O—C₁-C₄-alkyl, or —C(O)—C₁-C₄-alkyl;
A represents 5- to 10-membered heteroaryl which is optionally substituted with one or more substituents, which are the same or different, and selected from the group consisting of a halogen atom, C₁-C₃-alkyl, and C₁-C₃-alkoxy, wherein C₁-C₃-alkyl and C₁-C₃-alkoxy are optionally substituted with 1-5 halogen atoms which are the same or different;
q represents an integer of 0, 1, or 2;
   or an isomer, enantiomer, diastereomer, racemate, hydrate, solvate, or a salt thereof, or a mixture of same can be used for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

By providing said treatment options, it is possible to solve the problem of significant side effects known from present SoC (standard of care) therapies for cardiovascular diseases (CVD), and hypertension.

The prevention of additional significant side effects to important physiological functions, i.e. taste, wakefulness, or heart rate, that may weaken the potential clinical effectiveness of medicaments, is an advantage of this invention.

That means, for example, the avoidance of negatively effecting important physiological functions, like taste sensation, the avoidance of physical dependency, increased heart rate, xerostomia, constipation, nausea, drowsiness or sedation, which all have severe impact to the quality of life of patients. This makes it possible to provide a for the treatment of breathing disorders, Cheyne Stokes respiration, Central and obstructive sleep apnea, cardiovascular disease, hypertension, resistant hypertension, and heart failure which is usable for a chronic treatment of the mentioned diseases. Furthermore, the oral treatment of breathing disorders, Cheyne Stokes respiration, Central and obstructive sleep apnea, cardiovascular disease, hypertension, resistant hypertension, and heart failure, is possible with the provided treatment approach.

The present invention is based on the discovery that compounds of general formula (I) are highly potent and sufficient selective at the P2X3 receptor. Therefore the subject matter of the present invention is directed to the use of compounds of general formula (I) for the treatment or prophylaxis of diseases or disorders, which are associated with nerve fiber sensitization and/or associated with autonomic imbalance. The autonomic imbalance can be caused by increased chemoreceptor sensitivity.

In accordance with a first aspect, the present invention covers the use of compounds of general formula (I) for the treatment or prophylaxis of breathing disorders, Cheyne Stokes respiration, Central and obstructive sleep apnea, cardiovascular disease, hypertension, resistant hypertension, and heart failure.

In accordance with a second aspect, the present invention relates to the use of compounds of general formula (I) for long-term treatment of breathing disorders, Cheyne Stokes respiration, Central and obstructive sleep apnea, cardiovascular disease, hypertension, resistant hypertension, and heart failure.

In accordance with a third aspect, the present invention relates to the use of compounds of general formula (I) for oral treatment of breathing disorders, Cheyne Stokes respiration, Central and obstructive sleep apnea, cardiovascular disease, hypertension, resistant hypertension, and heart failure.

In accordance with a fourth aspect, the present invention relates to the use of compounds of general formula (I) for long-term and oral treatment of breathing disorders, Cheyne Stokes respiration, Central and obstructive sleep apnea, cardiovascular disease, hypertension, resistant hypertension, and heart failure.

In accordance with a fifth aspect, the present invention relates to the use of compounds of general formula (I) for long-term and oral treatment of breathing disorders, Cheyne Stokes respiration, Central and obstructive sleep apnea, cardiovascular disease, hypertension, resistant hypertension, and heart failure.

In accordance with a seventh aspect, the present invention covers a method of treatment or prophylaxis of breathing disorders, Cheyne Stokes respiration, Central and obstructive sleep apnea, cardiovascular disease, hypertension, resistant hypertension, and heart failure in a subject in need thereof.

In accordance with an eighth aspect, the present invention covers a method of long-term treatment of breathing disorders, Cheyne Stokes respiration, Central and obstructive sleep apnea, cardiovascular disease, hypertension, resistant hypertension, and heart failure in a subject in need thereof.

In accordance with a ninth aspect, the present invention covers a method of oral treatment of breathing disorders, Cheyne Stokes respiration, Central and obstructive sleep apnea, cardiovascular disease, hypertension, resistant hypertension, and heart failure in a subject in need thereof.

In accordance with a tenth aspect, the present invention covers a method of long-term and oral treatment of breathing disorders, Cheyne Stokes respiration, Central and obstructive sleep apnea, cardiovascular disease, hypertension, resistant hypertension, and heart failure in a subject in need thereof.

In accordance with an eleventh aspect, the present invention covers a method of long-term and oral treatment of breathing disorders, Cheyne Stokes respiration, Central and obstructive sleep apnea, cardiovascular disease, hypertension, resistant hypertension, and heart failure in a subject in need thereof.

A method in accordance with the invention comprises administering to the subject in need thereof an effective amount of a compound of formula (I) or a pharmaceutically acceptable salt thereof. The method comprises administering an effective amount of a compound of Formula (I).

The present invention further relates to the use of compounds of general formula (Ia),

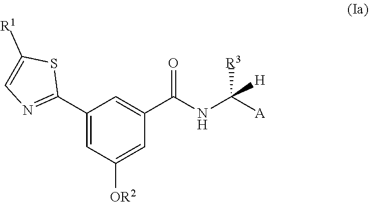

in which A, R¹, R² and R³ have the meanings as defined in formula (I), preferably R³ represents C₁-C₄-alkyl, more preferably methyl;
or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

The present invention further relates to the use of pharmaceutical compositions and combinations comprising the compounds of general formula (I) for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

The present invention further relates to the use of pharmaceutical compositions and combinations comprising the compounds of general formula (I) for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

### DESCRIPTION OF FIGURES

FIG. 1 shows the breathing rate response of anesthetized adult male Sprague Dawley rats to acute hypocapnic hypoxia by compounds of general formula (I), i.e. patent example 348 as described in WO2016/091776 in comparison with Afferent's AF-219. Pictured here is the breathing rate of anesthetized male Sprague Dawley rats as measured by esophageal catheter under normoxia (21% Oxygen) and during hypoxic (12% Oxygen) challenge. Compound causes a lowering of baseline breathing rate and a blunted response to hypoxia are observed in rats treated with P2X3 inhibitor, i.e. compounds of general formula (I), i.e. patent example 348 as described in WO2016/091776 in comparison with Afferent's AF-219.

FIG. 2 shows the baseline ventilation in conscious animals by whole-body plethysmography (respiration measured by whole body plethysmography in SHR). Animals were treated with compounds of general formula (I), i.e. patent example 11 as described in WO2016/091776 p.o. before being placed into plethysmography chambers. The data shown are the average of 30 minutes of continuous minute ventilation measurements from 1.5 to 2 hours after the start of the measurement. Data shown mean±SE. **, p<0.01.

FIG. 3 shows the ventilatory response in conscious animals by whole-body plethysmography in Sprague dawley rats. Animals were treated with compounds of general formula (I), i.e. patent example 348 as described in WO2016/091776 p.o. before being placed into plethysmography chambers. Compound was administered 3 hours before initiation of a 10 minute hypoxic challenge (10% O₂ balanced with N₂). The data shown are the area under the curve during the last 5 minutes of hypoxic challenge 95-100 minutes after the start of the measurement. Data shown are mean±SE. *, p<0.05; **, p<0.01

FIG. 4 shows the blood pressure monitoring in conscious animals by radiotelemetry (percent deviation of mean arterial pressure (MAP) in SHR). Compound or vehicle was given p.o. at time zero. Data shown are 30 minute averages over a 24 hour period. Lower MAP is observed in SHR treated with P2X3 inhibitors i.e. compounds of general formula (I), i.e. patent example 348 as described in WO2016/091776.

### DETAILED DESCRIPTION OF THE INVENTION

The terms as mentioned in the present text have preferably the following meanings:

The term “halogen atom”, “halo-” or “Hal-” is to be understood as meaning a fluorine, chlorine, bromine or iodine atom, preferably a fluorine or a chlorine atom.

The term “alkyl” is to be understood as meaning a linear or branched, saturated, monovalent hydrocarbon group with the number of carbon atoms as specified and having as a rule, 2 to 6 in case of R², and 1 to 4 for all other alkyl substituents, preferably 1 to 3, carbon atoms, by way of example and by preference a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, n-propyl, n-butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group, and even more particularly 1 or 2 carbon atoms (“C₁-C₂-alkyl”), e.g. a methyl or ethyl group.

The term “C₁-C₄-alkyl, optionally substituted with 1-5 halogen atoms”, or in analogy “C₁-C₃-alkyl, optionally substituted with 1-5 halogen atoms” or “C₁-C₂-alkyl which are optionally substituted with 1-5 halogen atoms”, is to be understood as meaning a linear or branched, saturated, monovalent hydrocarbon group in which the term “C₁-C₄-alkyl”, “C₁-C₃-alkyl” or “C₁-C₂-alkyl” is defined supra, and in which one or more hydrogen atoms is replaced by a halogen atom, which are the same or different, i.e. one halogen atom being independent from another. In particular, halogen is fluorine or chlorine.

The term “C₁-C₄-alkyl, optionally substituted with 1-5 fluorine atoms”, or in analogy “C₁-C₃-alkyl, optionally substituted with 1-5 fluorine atoms” or “C₁-C₂-alkyl, optionally substituted with 1-5 fluorine atoms”, is to be understood as meaning a linear or branched, saturated, monovalent hydrocarbon group in which the term “C₁-C₄-alkyl”, “C₁-C₃-alkyl” or “C₁-C₂-alkyl” is defined supra, and in which one or more hydrogen atoms is replaced by a fluorine atom.

Said “C₁-C₄-alkyl, optionally substituted with 1-5 fluorine atoms” or “C₁-C₄-alkyl group, optionally substituted with 1-5 halogen atoms” is, for example,

—CH₂CH₂CH₂CF₃.

Similarly, the above-mentioned applies to “C₁-C₃-alkyl, optionally substituted with 1-5 halogen atoms”, or “C₁-C₂-alkyl, optionally substituted with 1-5 halogen atoms”, or “C₁-C₃-alkyl, optionally substituted with 1-5 fluorine atoms”, or “C₁-C₂-alkyl, optionally substituted with 1-5 fluorine atoms”. Thus said “C₁-C₃-alkyl optionally substituted with 1-5 halogen atoms” or “C₁-C₃-alkyl optionally substituted with 1-5 fluorine atoms” is, for example,

—CH₂CH₂CF₃.

Said “C₁-C₂-alkyl optionally substituted with 1-5 halogen atoms” or “C₁-C₂-alkyl optionally substituted with 1-5 fluorine atoms” is, for example, —CF₃, —CHF₂, —CH₂F, —CF₂CF₃, —CH₂CHF₂, or —CH₂CF₃.

Under the proviso, that R² in formula (I) or (a) is —C₂-C₆-alkyl-OR⁴, “C₂-C₆-alkyl” is to be understood as C₁-C₅-alkylene which is bound to the phenolic oxygen via —CH₂— group. For example C₁-C₅-alkylene is methylene, ethylene, propylene, butylene, pentylene, iso-propylene, iso-butylene, sec-butylene, tert-butylene, iso-pentylene, 2-methylbutylene, 1-methylbutylene, 1-ethylpropylene, 1,2-dimethylpropylene, neo-pentylene, 1,1-dimethylpropylene.

Under the proviso, that R² in formula (I) or (a) is —C₂-C₆-alkyl-OR⁴, “C₂-C₆-alkyl” is also to be understood as C₁-C₄-alkylene which is bound to the phenolic oxygen via —CH—CH₃ group.

Under the proviso, that R² in formula (I) or (a) is —C₂-C₄-alkyl-OR⁴, “C₂-C₄-alkyl” is to be understood as C₁-C₃-alkylene which is bound to the phenolic oxygen via —CH₂— group. Under the proviso that R² in formula (I) or (a) is —C₂-C₄-alkyl-OR⁴, “C₂-C₄-alkyl” is also to be understood as C₁-C₂-alkylene which is bound to the phenolic oxygen via —CH—CH₃ group.

Under the proviso, that R² in formula (I) or (a) is —C₂-C₄-alkyl-OH, “C₂-C₄-alkyl” is to be understood as C₁-C₃-alkylene which is bound to the phenolic oxygen via —CH₂— group. Under the proviso that R² in formula (I) or (a) is —C₂-C₄-alkyl-OH, “C₂-C₄-alkyl” is also to be understood as C₁-C₂-alkylene which is bound to the phenolic oxygen via —CH—CH₃ group.

Under the proviso, that R² in formula (I) or (a) is —C₂-C₆-alkyl-OR⁴, “—OR⁴” is either at a tertiary, secondary or primary carbon atom of the —C₂-C₆-alkyl chain.

Under the proviso, that R² in formula (I) or (a) is —C₂-C₄-alkyl-OR⁴, “—OR⁴” is either at a tertiary, secondary or primary carbon atom of the —C₂-C₄-alkyl chain.

Under the proviso, that R² in formula (I) or (a) is —C₂-C₄-alkyl-OH, “—OH” is either at a tertiary, secondary or primary carbon atom of the —C₂-C₄-alkyl chain.

For example, said —C₂-C₆-alkyl-OR⁴ is 3-hydroxybutan-2-yl, (2R,3R)-3-hydroxybutan-2-yl, (2S,3S)-3-hydroxybutan-2-yl, (2R,3S)-3-hydroxybutan-2-yl, (2S,3R)-3-hydroxybutan-2-yl, (2R,3R)-3-methoxybutan-2-yl, (2S,3S)-3-methoxybutan-2-yl, (2R,3S)-3-methoxybutan-2-yl, (2S,3R)-3-methoxybutan-2-yl, 3-methoxybutan-2-yl, 2-hydroxy-2-methylpropan-1-yl, 2-methoxy-2-methylpropan-1-yl, 3-hydroxypropan1-yl, 3-hydroxybutan-1-yl, 3-hydroxy-3-methylbutan-1-yl, 3-hydroxy-2-methylbutan-1-yl, 3-hydroxy-2,2-dimethylpropan-1-yl, 4-hydroxy-3-methylbutan-2-yl, 4-hydroxy-3-methylpent-1-yl, 4-hydroxy-4-methylpent-1-yl, 2-hydroxy-2-methylpropan-1-yl, 2-methoxy-2-methyl-propan-1-yl, 2-methoxyethan-1-yl, 3-methoxypropan-1-yl, 4-methoxybutan-1-yl, 2-ethoxyethan-1-yl, 3-ethoxypropan-1-yl, 4-ethoxybutan-1-yl, 2-iso-propoxyethan-1-yl, 3-iso-propoxypropan-1-yl, 4-iso-propoxybutan-1-yl, 2-hydroxyethan-1-yl, 3-hydroxy-propan-1-yl, 4-hydroxybutan-1-yl, preferably 3-hydroxybutan-2-yl, (2R,3R)-3-hydroxybutan-2-yl, (2S,3S)-3-hydroxybutan-2-yl, (2R,3S)-3-hydroxybutan-2-yl, (2S,3R)-3-hydroxybutan-2-yl, more preferably (2R,3R)-3-hydroxybutan-2-yl, (2S,3S)-3-hydroxybutan-2-yl.

For example, said —C₂-C₄-alkyl-OR⁴ or —C₂-C₄-alkyl-OH is preferably 3-hydroxybutan-2-yl, (2R,3R)-3-hydroxybutan-2-yl, (2S,3S)-3-hydroxybutan-2-yl, (2R,3S)-3-hydroxybutan-2-yl, (2S,3R)-3-hydroxybutan-2-yl, more preferably (2R,3R)-3-hydroxybutan-2-yl, (2S,3S)-3-hydroxybutan-2-yl.

The term “alkoxy” is to be understood as meaning a linear or branched, saturated, monovalent, hydrocarbon group of formula —O-alkyl, in which the term “alkyl” is defined as meaning a linear or branched, saturated, monovalent hydrocarbon group with the number of carbon atoms as specified and having as a rule, 1 to 3, preferably 1 to 2 alkyl substituents, especially preferably 1, carbon atoms. Particularly, said group has 1, 2 or 3 carbon atoms (“C₁-C₃-alkoxy”), e.g. a methoxy, ethoxy, n-propoxy or iso-propoxy group, and even more particularly 1 or 2 carbon atoms (“C₁-C₂-alkoxy”), e.g. a methoxy or ethoxy group.

The term “C₁-C₃-alkoxy optionally substituted with 1-5 halogen atoms” is to be understood as meaning a linear or branched, saturated, monovalent hydrocarbon group in which the term “C₁-C₃-alkoxy” is defined supra, and in which one or more hydrogen atoms is replaced by a halogen atom, which are the same or different, i.e. one halogen atom being independent from another. In particular, halogen is fluorine or chlorine.

Said “C₁-C₃-alkoxy” group is optionally substituted with 1 to 5 fluorine atoms, for example, —OCF₃, —OCHF₂, —OCH₂F, —OCF₂CF₃, —OCH₂CHF₂, —OCH₂CF₃, —OCH₂CH₂CF₃, or —OCH₂CF₂CF₃. In particular, said “C₁-C₃-alkoxy” group optionally substituted with fluorine is —OCF₃.

The term “C₂-C₆-alkynyl” is to be understood as meaning a linear or branched, monovalent hydrocarbon group which contains one or more triple bonds, preferably one triple bond, and which contains 2, 3, 4, 5 or 6 carbon atoms, particularly 3 or 4 carbon atoms (“C₃-C₄-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent2-ynyl, 1-methyl-pent-2-ynyl, 4-methylpent-1-ynyl, 3-methylpent-1-ynyl, 2-ethylbut-3-ynyl, 1-ethyl-but-3-ynyl, 1-ethylbut-2-ynyl, 1-propylprop-2-ynyl, 1-isopropylprop-2-ynyl, 2,2-di-methylbut-3-ynyl, 1,1-dimethylbut-3-ynyl, 1,1-dimethylbut-2-ynyl, or 3,3-dimethyl-but-1-ynyl group. Particularly, said alkynyl group is prop-1-ynyl or prop-2-ynyl.

The term “cycloalkyl” is to be understood as meaning a saturated, monovalent, monocyclic hydrocarbon ring with the number of carbon atoms as specified and having as a rule, 3 to 7 or 3 to 6 ring carbon atoms, preferably 3 to 4 ring carbon atoms.

“C₃-C₇-cycloalkyl” is to be understood as meaning a saturated, monovalent, monocyclic hydrocarbon ring which contains 3, 4, 5, 6 or 7 carbon atoms. Said C₃-C₇-cycloalkyl group is for example a cyclopropyl, cyclobutyl, cyclopentyl, cyclohexyl or cycloheptyl ring. Each hydrogen of a cycloalkyl carbon may be replaced by a substituent as further specified. Particularly, said ring contains 3, 4, 5 or 6 carbon atoms (“C₃-C₆-cycloalkyl”), preferably 3 or 4 carbon atoms (“C₃-C₄-cycloalkyl”).

In case of R² in formula (I) or (Ia), said “C₃-C₇-cycloalkyl” in “(CH₂)_{q}—(C₃-C₇-cycloalkyl)” is, unless indicated otherwise, optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O). In case of R² in formula (I) or (Ia), said “C₃-C₄-cycloalkyl” as such or “C₃-C₄-cycloalkyl” in “CH₂—(C₃-C₄-cycloalkyl)” is, unless indicated otherwise, optionally substituted with one or more substituents which are the same or different, at any ring carbon atom and selected from a group consisting of C₁-C₄-alkyl, optionally substituted with 1-5 halogen atoms which are the same or different, a halogen atom, —NR^{a}R^{b}, —COOR⁵ and oxo (═O).

The term “heterocycloalkyl” is to be understood as meaning a saturated, monovalent, monocyclic hydrocarbon ring with the number of ring atoms as specified in which one, two or three ring atoms of the hydrocarbon ring is/are replaced by one, two or three heteroatoms or heteroatom-containing groups independently selected from O, S, S(═O), S(═O)₂, or N.

“4- to 7-membered heterocycloalkyl” is to be understood as meaning a saturated, monovalent, monocyclic “heterocycloalkyl” ring as defined supra which contains 4, 5, 6 or 7 ring atoms.

Similarly, “4- to 6-membered heterocycloalkyl” is to be understood as meaning a saturated, monovalent, monocyclic “heterocycloalkyl” ring as defined supra which contains 4, 5 or 6 ring atoms.

In case of R² in formula (I) or (Ia), said 4- to 7-membered heterocycloalkyl or 4- to 6-membered heterocycloalkyl is, unless indicated otherwise, optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O); and wherein independently any ring nitrogen atom, if present in said 4- to 7-membered or 4- to 6-membered heterocycloalkyl is substituted with R^{c}; it being possible for said 4- to 7-membered or 4- to 6-membered heterocycloalkyl group to be attached to the rest of the molecule via any one of the carbon atoms or, if present, a nitrogen atom. Accordingly, any ring nitrogen atom if present in said 4- to 7-membered or 4- to 6-membered heterocycloalkyl group is only substituted with R^{c}, if the designated atom's normal valency under the existing circumstances is not exceeded.

Particularly, said 4- to 7-membered heterocycloalkyl can contain 3, 4, 5 or 6 carbon atoms, and one or two of the above-mentioned heteroatoms or heteroatom-containing groups provided that the total number of ring atoms is not greater than 7, more particularly said heterocycloalkyl can contain 3, 4 or 5 carbon atoms, and one or two of the above-mentioned heteroatoms or heteroatom-containing groups provided that the total number of ring atoms is not greater than 6 (a “4- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, or a 6-membered ring, such as tetrahydropyranyl, piperidinyl, morpholinyl, dithianyl, thiomorpholinyl, piperazinyl, or a 7-membered ring, such as a diazepanyl ring, for example.

Particularly, without being limited thereto, said heterocycloalkyl can be in a more preferred embodiment (3R)-tetrahydrofuran-3-yl, (3S)-tetrahydrofuran-3-yl, 4-methylmorpholin-2-yl, (2R)-4-methylmorpholin-2-yl, (2S)-4-methylmorpholin-2-yl, 4-methylmorpholin-3-yl, (3R)-4-methylmorpholin-3-yl, or (3S)-4-methylmorpholin-3-yl, most preferred (2R)-4-methylmorpholin-2-yl.

The term “6- to 12-membered heterobicycloalkyl” is to be understood as meaning a saturated, monovalent bicyclic hydrocarbon radical in which the two rings share one or two common ring atoms, and wherein said bicyclic hydrocarbon radical contains 5, 6, 7, 8, 9 or 10 carbon atoms and one, two or three heteroatoms or heteroatom-containing groups independently selected from O, S, S(═O), S(═O)₂, or N, provided that the total number of ring atoms is not greater than 12. Said 6- to 12-membered heterobicycloalkyl is, unless indicated otherwise, optionally substituted with one or more substituents, which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵ and oxo (═O); and wherein independently any ring nitrogen atom, if present in said 6- to 12-membered heterobicycloalkyl is substituted with R^{c}; it being possible for said 6- to 12-membered heterobicycloalkyl to be attached to the rest of the molecule via any one of the carbon atoms or, if present, a nitrogen atom. Accordingly, any ring nitrogen atom if present in said 6- to 12-membered heterobicycloalkyl is only substituted with Rc, if the designated atom's normal valency under the existing circumstances is not exceeded. Said 6- to 12-membered heterobicycoalkyl is, for example, azabicyclo[3.3.0]octyl, azabicyclo-[4.3.0]nonyl, diazabicyclo[4.3.0]nonyl, oxazabicyclo[4.3.0]nonyl, thiazabicyclo-[4.3.0]nonyl or azabicyclo[4.4.0]decyl.

Heterospirocycloalkyl and bridged heterocycloalkyl, as defined below, are also included within the scope of this definition.

The term “heterospirocycloalkyl” is to be understood as meaning a saturated, monovalent bicyclic hydrocarbon radical in which the two rings share one common ring atom, and wherein said bicyclic hydrocarbon radical contains 5, 6, 7, 8, 9 or 10 carbon atoms, and one, two or three heteroatoms or heteroatom-containing groups independently selected from O, S, S(═O), S(═O)₂, or N, provided that the total number of ring atoms is not greater than 12. It is possible for said heterospirocycloalkyl to be attached to the rest of the molecule via any one of the carbon atoms or, if present, a nitrogen atom. Said heterospirocycloalkyl is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, oxazaspiro[5.5]undecyl, diazaspiro[3.3]heptyl, thiazaspiro[3.3]heptyl, thiazaspiro[4.3]octyl, or azaspiro[5.5]decyl.

The term “bridged heterocycloalkyl” is to be understood as meaning a saturated, monovalent bicyclic hydrocarbon radical in which the two rings share two common ring atoms which are not immediately adjacent, and wherein said bicyclic hydrocarbon radical contains 5, 6, 7, 8, 9 or 10 carbon atoms, and one, two or three heteroatoms or heteroatom-containing groups independently selected from O, S, S(═O), S(═O)₂, or N, provided that the total number of ring atoms is not greater than 12. It is possible for said bridged heterocycloalkyl to be attached to the rest of the molecule via any one of the carbon atoms or, if present, a nitrogen atom. Said bridged heterocycloalkyl is, for example, azabicyclo[2.2.1]heptyl, oxazabicyclo[2.2.1]heptyl, thiazabicyclo[2.2.1]heptyl, diazabicyclo[2.2.1]heptyl, azabicyclo[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo-[3.2.1]octyl, thiazabicyclo[3.2.1]octyl, azabicyclo[3.3.1]nonyl, diazabicyclo[3.3.1]-nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl, or azabicyclo[4.2.2]decyl.

The term “heteroaryl” is understood as meaning a monovalent, monocyclic or bicyclic hydrocarbon ring system with at least one aromatic ring with the number of ring system atoms as specified and wherein one, two or three ring atoms of the monovalent, monocyclic or bicyclic hydrocarbon ring system is/are replaced by one, two or three heteroatoms or heteroatom-containing groups independently selected from O, S, S(═O), S(═O)₂, or N.

“5- to 10-membered heteroaryl” is understood as meaning a heteroaryl having 5, 6, 7, 8, 9 or 10 ring atoms (a “5- to 10-membered heteroaryl”) and wherein one, two or three ring atoms of the monovalent, monocyclic or bicyclic hydrocarbon ring system is/are replaced by one, two or three heteroatoms or heteroatom-containing groups independently selected from O, S, S(═O), S(═O)₂, or N. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc. and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridinyl, pyridazinyl, pyrimidinyl, pyrazinyl, triazinyl, etc., and benzo derivatives thereof, such as, for example, quinolinyl, quinazolinyl, isoquinolinyl, etc.; indolizinyl, and benzo derivatives thereof; or cinnolinyl, phthalazinyl, quinazolinyl, quinoxalinyl, etc.

In case of R² of formula (I) or (Ia), said 5- to 10-membered heteroaryl is, unless indicated otherwise, optionally substituted with one or more substituents which are the same or different, and selected from the group consisting of C₁-C₄-alkyl, optionally substituted with 1-5 halogen atoms which are the same or different, a halogen atom, —NR^{a}R^{b} and —COOR⁵.

In case of R² of formula (I) or (Ia), said 5- to 10-membered heteroaryl optionally substituted as described above, can be in particular substituted with C₁-C₂-alkyl at any ring N, if present.

In case of A of formula (I) or (Ia), said 5- to 10-membered heteroaryl is, unless indicated otherwise, optionally substituted with one or more substituents, which are the same or different, and selected from the group consisting of a halogen atom, C₁-C₃-alkyl, and C₁-C₃-alkoxy, wherein C₁-C₃-alkyl and C₁-C₃-alkoxy are optionally substituted with 1-5 halogen atoms which are the same or different.

In case of A of formula (I) or (Ia), a “5- or 6-membered heteroaryl” is understood as meaning a heteroaryl having 5 or 6 ring atoms and wherein one, two or three ring atoms of the hydrocarbon ring system is/are replaced by one, two or three heteroatoms or heteroatom-containing groups independently selected from O, S, S(═O), S(═O)₂, or N. Said “5- or 6-membered heteroaryl” is, unless indicated otherwise, optionally substituted with one or more substituents, which are the same or different, and selected from the group consisting of a halogen atom, C₁-C₃-alkyl, and C₁-C₃-alkoxy, wherein C₁-C₃-alkyl and C₁-C₃ alkoxy are optionally substituted with 1-5 halogen atoms which are the same or different

A 5-membered heteroaryl group is preferably selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl.

A 6-membered heteroaryl group is preferably selected from pyridinyl, pyridazinyl, pyrimidinyl, pyrazinyl, triazinyl.

In particular, said 5- or 6-membered heteroaryl is, optionally substituted with preferably one or two substituents, which are the same or different, and selected from a fluorine or chlorine atom, C₁-C₂-alkyl, optionally substituted with 1-5 fluorine atoms, or C₁-C₂-alkoxy, optionally substituted with 1-5 fluorine atoms.

In particular, said 5- or 6-membered heteroaryl is a 6-membered heteroaryl with one or two nitrogen atom(s) and is optionally substituted with one or two substituents, which are the same or different, and selected from a fluorine or chlorine atom, C₁-C₂-alkyl, optionally substituted with 1-5 fluorine atoms, or C₁-C₂-alkoxy, optionally substituted with 1-5 fluorine atoms.

Preferably said 6-membered heteroaryl is CF₃-pyrimidinyl, most preferably 2-CF₃-pyrimidin-5-yl. Also preferred is CF₃-pyridazinyl, most preferably 6-CF₃-pyridazin-3-yl.

In general, and unless otherwise mentioned, the term “heteroaryl” includes all possible isomeric forms thereof, e.g. the positional isomers thereof. Thus, for some illustrative non-restricting example, the term pyridyl includes pyridin-2-yl, pyridin-3-yl, and pyridin-4-yl; or the term pyrimidinyl includes pyrimidin-2-yl, pyrimidin-4-yl and pyrimidin-5-yl; or the term pyridazinyl includes pyridazin-3-yl and pyridazin-4-yl; or the term thiazolyl includes 1,3-thiazol-5-yl, 1,3-thiazol-4-yl and 1,3-thiazol-2-yl.

The term “C₁-C₄” as used throughout this text is to be understood as meaning a group having a finite number of carbon atoms of 1 to 4, i.e. 1, 2, 3 or 4 carbon atoms, e.g. in the context of the definition of “C₁-C₄-alkyl”, it is to be understood as meaning an alkyl group having a finite number of carbon atoms of 1 to 4, i.e. 1, 2, 3 or 4 carbon atoms.

The term “C₂-C₆” as used throughout this text is to be understood as meaning a group having a finite number of carbon atoms of 2 to 6, i.e. 2, 3, 4, 5 or 6 carbon atoms, e.g. in the context of the definition of “C₂-C₆-alkyl”, it is to be understood as meaning an alkyl group having a finite number of carbon atoms of 2 to 6, i.e. 2, 3, 4, 5 or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

The term “C₁-C₃” as used in the context of the definition “C₁-C₃-alkoxy” is to be understood as meaning an alkoxy group, having a finite number of carbon atoms of 1 to 3, i.e. 1, 2 or 3 carbon atoms.

The same applies to other mentioned “alkyl”, alkynyl or “alkoxy” as mentioned herein and as it is to be understood by a skilled person.

It is to be understood further that for example a term “C₁-C₆” is to be interpreted as any sub-range comprised therein, e.g. C₁-C₆, C₂-C₃, C₂-C₆, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄.

Similarly, the mentioned above applies to “C₁-C₄-alkyl”, “C₁-C₃-alkyl”, “C₁-C₃-alkoxy”, “C₁-C₂-alkyl” or “C₁-C₂-alkoxy” optionally substituted with 1-5 halogen which are the same or different.

Similarly, as used herein, the term “C₂-C₆”, as used throughout this text, e.g. in the context of the definitions of “C₂-C₆-alkynyl”, is to be understood as meaning an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃ and C₂-C₄.

Further, as used herein, the term “C₃-C₇”, as used throughout this text, is to be understood as meaning a group having a finite number of carbon atoms of 3 to 7, i.e. 3, 4, 5, 6 or 7 carbon atoms, e.g. in the context of the definition of “C₃-C₇-cycloalkyl”, it is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 5, in particular from 1 to 3.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, sulphur, fluorine and chlorine such as ²H (deuterium), ³H (tritium), ¹¹C, ¹³C, ¹⁴C, ¹⁵N, ¹⁷O, ¹⁸O, ³³S, ³⁴S, ³⁵S, ³⁶S, ¹⁸F and ³⁶Cl, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active forms of compounds of formula (I) can likewise be obtained by chiral syntheses utilizing optically active starting materials.

In order to limit different types of isomers from each other reference is made to IUPAC Rules Section E (Pure Appl Chem 45, 11-30, 1976).

Further, compounds may exist as tautomers.

The present compounds of formula (I) includes all possible tautomers as single tautomers, or as any mixture of said tautomers, in any ratio.

The present invention also relates to the use of useful forms of compounds of formula (I), such as metabolites, hydrates, solvates, prodrugs, salts, in particular pharmaceutically acceptable salts, and co-precipitates.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

Compounds of formula (I) can exist as a hydrate, or as a solvate, wherein compounds of formula (I) contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present compounds include all such hydrates or solvates.

Further, compounds of formula (I) can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of compounds of formula (I). For example, see S. M. Berge, et al. “Pharmaceutical Salts,” J. Pharm. Sci. 1977, 66, 1-19. A suitable pharmaceutically acceptable salt of the compounds of the present invention may be, for example, an acid-addition salt of compounds of formula (I) bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethanesulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of formula (I) which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of compounds of formula (I) may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of compounds of formula (I) as single salts, or as any mixture of said salts, in any ratio.

Unless otherwise indicated, compounds of formula (I) are also referred to isomers, enantiomers, diastereomers, racemates, hydrates, solvates, or a salt thereof, or a mixture of same.

As used herein, the term “in vivo hydrolysable ester” is understood as meaning an in vivo hydrolysable ester of a compound of formula (I) containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of formula (I). An in vivo hydrolysable ester of a compound of formula (I) containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of compounds of formula (I), either as single polymorphs, or as a mixture of more than one polymorph, in any ratio.

Heart failure is defined by the European Socient of Cardiology treatment guidelines as a symptomatic clinical manifestation of heart systolic or diastolic functional abnormalities resulting in reduced cardiac output and/or elevated intracardiac pressures at rest or during stress. Systolic and/or diastolic functional abnormalities can also result from genetic predisposition, abnormalities in the myocardium, valves, endocardium, pericardium and conduction system of the heart. Heart failure with preserved, mid-range, and reduced ejection fraction are defined as ejection fractions >50%, 40-49%, and <40% respectively (Ponikowski et al. Eur Heart J. 2016.37:27(2129-200)).

Hypertension is the most common cardiovascular disease comorbidity and is clearly associated with increased event rates in cardiovascular disease and heart failure. Hypertension is defined by the European Society of Hypertension as arterial systolic blood pressure values >140 mmHg and/or diastolic blood pressure values >90 mmHg (Mancia et al. J Hypertens 2013. 31:7(1281-357)).

Cheyne-Stokes respiration is an abnormal breathing pattern that can occur during sleep or wakefulness. Cheyne-Stokes respiration is characterized by a periodic crescendo/decreshendo breathing pattern—gradually increasing speed and depth of breathing followed by a decrease in depth and frequency of breathing. This pattern also results in periodic apnea and cessation of breathing.

Central sleep apnea is characterized by periodic diminished or lack of respiratory effort during sleep. It is normally associated with symptoms such as frequent arousal during sleep and daytime sleepiness or both.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

“Treating” or “treatment” of a disease state includes: (i) inhibiting the disease state, i.e. arresting the development of the disease state or its clinical symptoms, or (ii) relieving the disease state, i.e. causing temporary or permanent regression of the diseasestate or its clinical symptoms.

“Preventing” or “prevention” of a disease state includes causing the clinical symptoms of the disease state not to develop in a subject that may be exposed to or predisposed to the disease state, but does not yet experience or display symptoms of the disease state. For example, treating or preventing a respiratory disease or disorder includes treating or preventing the symptoms the disorder such as cough and/or urge to cough associated with a respiratory disease.

Another embodiment of the present invention relates to a method for using compounds of general formula (Ia),

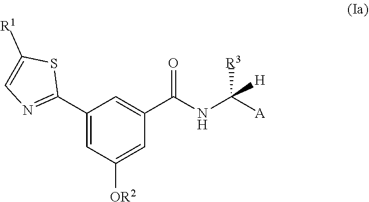

in which A, R¹, R² and R³ have the meanings as defined in formula (I), preferably R³ represents C₁-C₄-alkyl, more preferably methyl;
or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents an optionally substituted 5- or 6-membered heteroaryl, preferably an optionally substituted 6-membered heteroaryl; and
   in which R¹, R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents an optionally substituted 5- or 6-membered heteroaryl, preferably an optionally substituted 6-membered heteroaryl; and
   in which R¹, R² and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Additionally, an embodiment of the present invention relates to a method for using compounds of general formula (I), wherein
R¹ represents C₁-C₄-alkyl, preferably methyl or ethyl; and
   in which A, R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using are compounds of general formula (Ia), wherein
R¹ represents C₁-C₄-alkyl, preferably methyl or ethyl; and
   in which A, R² and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R¹ represents a halogen atom, preferably chloro; and
   in which A, R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R¹ represents a halogen atom, preferably chloro; and
   in which A, R² and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
R³ represents C₁-C₄-alkyl, preferably methyl; and
   in which R¹, R² and A have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and
   in which A, R^{c}, R¹ and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and
   in which A, R^{c}, R¹ and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and
   in which A, R^{c}, R¹ and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
q represents an integer of 0; and
   in which A, R^{c}, R¹ and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R² represents —(CH₂)_{q}-(4- to 6-membered heterocycloalkyl); and wherein (CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4 to 6-membered heterocycloalkyl) is substituted with R^{c}; and wherein —(CH₂)_{q}-(4- to 6-membered heterocycloalkyl) is preferably —(CH₂)_{q}-morpholinyl; and
q represents an integer of 1; and
   in which A, R^{c}, R¹ and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R² represents-(CH₂)_{q}-morpholinyl, wherein the ring nitrogen atom is substituted with R^{c}; and
R^{c} represents methyl;
q represents an integer of 1; and
   in which A, R¹ and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R² represents —(CH₂)_{q}-(4- to 6-membered heterocycloalkyl); and wherein (CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4 to 6-membered heterocycloalkyl) is substituted with R^{c}; and wherein —(CH₂)_{q}-(4- to 6-membered heterocycloalkyl) is preferably —(CH₂)_{q}-morpholinyl; and
q represents an integer of 1; and
   in which A, R^{c}, R¹ and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R² represents-(CH₂)_{q}-morpholinyl, wherein the ring nitrogen atom is substituted with R^{c}; and
R^{c} represents methyl;
q represents an integer of 1; and
   in which A, R¹ and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R² represents —C₂-C₄-alkyl-OH; and
   in which A, R¹ and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using to compounds of general formula (Ia), wherein
R² represents —C₂-C₄-alkyl-OH; and
   in which A, R¹ and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents an optionally substituted 5- or 6-membered heteroaryl, preferably an optionally substituted 6-membered heteroaryl; and
R¹ represents C₁-C₄-alkyl, preferably methyl or ethyl; and
   in which R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents an optionally substituted 5- or 6-membered heteroaryl, preferably an optionally substituted 6-membered heteroaryl; and
R¹ represents C₁-C₄-alkyl, preferably methyl or ethyl; and
   in which R² and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents an optionally substituted 5- or 6-membered heteroaryl, preferably an optionally substituted 6-membered heteroaryl; and
R¹ represents a halogen atom, preferably chloro; and
   in which R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents an optionally substituted 5- or 6-membered heteroaryl, preferably an optionally substituted 6-membered heteroaryl; and
R¹ represents a halogen atom, preferably chloro; and
   in which R² and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl; and
R³ represents C₁-C₄-alkyl, preferably methyl; and
   in which R¹ and R² have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄-alkyl, preferably methyl or ethyl; and
R³ represents C₁-C₄-alkyl, preferably methyl; and
   in which R² has the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents a halogen atom, preferably chloro; and
R³ represents C₁-C₄-alkyl, preferably methyl; and
   in which R² has the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄.alkyl, preferably methyl or ethyl;
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c};
R³ represents C₁-C₄-alkyl, preferably methyl; and
q represents an integer of 0,
   wherein R^{c} is defined as in formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄.alkyl, preferably methyl or ethyl;
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl; and
   wherein said —CH₂—(C₃-C₄-cycloalkyl), C₃-C₄-cycloalkyl and —(CH₂)_{q}-(4- to 6-membered heterocycloalkyl) are optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c}; and
R³ represents C₁-C₄-alkyl, preferably methyl; and
q represents an integer of 0,
   wherein R^{c} is defined as in formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄.alkyl, preferably methyl or ethyl;
R² represents —(CH₂)_{q}-(4- to 6-membered heterocycloalkyl); and wherein (CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4 to 6-membered heterocycloalkyl) is substituted with R^{c}; wherein —(CH₂)_{q}-(4- to 6-membered heterocycloalkyl) is preferably —(CH₂)_{q}-morpholinyl;
R³ represents C₁-C₄-alkyl, preferably methyl; and
q represents an integer of 1;
   wherein R^{c} is defined as in formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents C₁-C₄.alkyl, preferably methyl or ethyl;
R² represents —(CH₂)_{q}-morpholinyl, wherein the ring nitrogen atom is substituted with R^{c}; and
R^{c} represents methyl;
R³ represents C₁-C₄-alkyl, preferably methyl; and
q represents an integer of 1;
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents an optionally substituted 5- or 6-membered heteroaryl, preferably an optionally substituted 6-membered heteroaryl;
R¹ represents a halogen atom, preferably chloro;
R² represents —C₂-C₄-alkyl-OH, preferably 3-hydroxybutan-2-yl;
R³ represents C₁-C₄-alkyl, preferably methyl;
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl;
R² represents —C₂-C₃-alkyl-OR⁴, unsubstituted —CH₂—(C₃-C₄-cycloalkyl), unsubstituted C₃-C₄-cycloalkyl, unsubstituted (CH₂)_{q}-(4- to 6-membered heterocycloalkyl), or —C₂-C₄-alkynyl;
R³ represents methyl; and
q represents an integer of 0,
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl;
R² represents optionally substituted (CH₂)_{q}-(4- to 6-membered heterocycloalkyl), wherein —(CH₂)_{q}-(4- to 6-membered heterocycloalkyl) is optionally substituted with one or more substituents which are the same or different, at any ring carbon atom; and
   wherein independently any ring nitrogen atom, if present in said —(CH₂)_{q}-(4- to 6-membered heterocycloalkyl) is substituted with R^{c}; wherein —(CH₂)_{q}-(4- to 6-membered heterocycloalkyl) is preferably —(CH₂)_{q}-morpholinyl;
R³ represents methyl; and
q represents an integer of 1,
   wherein R^{c} is defined as in formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl;
R² represents —(CH₂)_{q}-morpholinyl, wherein the ring nitrogen atom is substituted with R^{c} as defined in formula (I), preferably substituted with methyl;
R³ represents methyl; and
q represents an integer of 1,
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably compounds of general formula (Ia), wherein
A represents 5- or 6-membered heteroaryl at least containing one or two nitrogen atom(s), preferably a 6-membered heteroaryl with one or two nitrogen atom(s),
   wherein said 5- or 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents a chloro atom;
R² represents —C₂-C₄-alkyl-OH, preferably 3-hydroxybutan-2-yl; and
R³ represents methyl,
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents pyrimidinyl, pyridazinyl, pyridinyl, pyrazinyl, thiazolyl or thiadiazolyl, preferably pyrimidinyl, pyridazinyl, thiazolyl or thiadiazolyl, more preferably pyrimidinyl, pyridazinyl or thiadiazolyl, wherein said pyrimidinyl, pyridazinyl, pyridinyl, pyrazinyl, thiazolyl and thiadiazolyl are optionally substituted; and
   in which R¹, R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

In another preferred embodiment, the invention relates to use of compounds of general formula (Ia), wherein
A represents pyrimidinyl, pyridazinyl, pyridinyl, pyrazinyl, thiazolyl or thiadiazolyl, preferably pyrimidinyl, pyridazinyl, thiazolyl or thiadiazolyl, more preferably pyrimidinyl, pyridazinyl or thiadiazolyl, wherein said pyrimidinyl, pyridazinyl, pyridinyl, pyrazinyl, thiazolyl and thiadiazolyl are optionally substituted; and
   in which R¹, R² and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents CF₃-pyrimidinyl, preferably 2-CF₃-pyrimidin-5-yl; and
   in which R¹, R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

In another preferred embodiment, the invention relates to compounds of general formula (Ia), wherein
A represents CF₃-pyrimidinyl, preferably 2-CF₃-pyrimidin-5-yl; and
   in which R¹, R² and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents CF₃-pyridazinyl, preferably 6-CF₃-pyridazin-3-yl; and
   in which R¹, R² and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents CF₃-pyridazinyl, preferably 6-CF₃-pyridazin-3-yl; and
   in which R¹, R² and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using to compounds of general formula (I), wherein
R² represents cyclopropylmethyl, tetrahydrofuran-3-yl, tetrahydrofuran-2-ylmethyl, tetrahydrofuran-3-ylmethyl, prop-2-yn-1-yl, but-2-yn-1-yl, oxetan-3-yl, tetrahydropyran-4-yl, tetrahydro-2H-pyran-4-ylmethyl, pyridin-4-yl, pyridin-3-yl, 1,3,4-thiadiazol-2-yl, 1,3-thiazol-2-yl, 2,2-dimethyl-2-methoxyethyl, methoxyethyl, piperidin-4-yl, pyrrolidin-3-yl or azetidin-3-yl which are optionally substituted, preferably unsubstituted cyclopropylmethyl, unsubstituted oxetan-3-yl, unsubstituted tetrahydrofuran-3-yl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
R² represents 3-hydroxybutan-2-yl, prop-2-yn-1-yl, but-2-yn-1-yl, 2,2-dimethyl-2-methoxyethyl, methoxyethyl; or
   cyclopropylmethyl, tetrahydrofuran-3-yl, tetrahydrofuran-2-ylmethyl, tetrahydrofuran-3-ylmethyl, oxetan-3-yl, tetrahydropyran-4-yl, tetrahydro-2H-pyran-4-ylmethyl, (4-methylmorpholin-2-yl)methyl, pyridin-4-yl, pyridin-3-yl, 1,3,4-thiadiazol-2-yl, 1,3-thiazol-2-yl, piperidin-4-yl, pyrrolidin-3-yl or azetidin-3-yl which are optionally substituted,
   preferably unsubstituted cyclopropylmethyl, unsubstituted oxetan-3-yl, unsubstituted (3R)-tetrahydrofuran-3-yl, unsubstituted (3S)-tetrahydrofuran-3-yl, [(2R)-4-methylmorpholin-2-yl]methyl, [(2S)-4-methylmorpholin-2-yl]methyl, (2R,3R)-3-hydroxybutan-2-yl, (2S,3S)-3-hydroxybutan-2-yl, (2S,3R)-3-hydroxybutan-2-yl or (2R,3S)-3-hydroxybutan-2-yl; and
      in which R¹, A and R³ have the same meaning as defined in general formula (I),
      or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R² represents cyclopropylmethyl, tetrahydrofuran-3-yl, tetrahydrofuran-2-ylmethyl, tetrahydrofuran-3-ylmethyl, prop-2-yn-1-yl, but-2-yn-1-yl, oxetan-3-yl, tetrahydropyran-4-yl, tetrahydro-2H-pyran-4-ylmethyl, pyridin-4-yl, pyridin-3-yl, 1,3,4-thiadiazol-2-yl, 1,3-thiazol-2-yl, 2,2-dimethyl-2-methoxyethyl, methoxyethyl, piperidin-4-yl, pyrrolidin-3-yl or azetidin-3-yl which are optionally substituted, preferably unsubstituted cyclopropylmethyl, unsubstituted oxetan-3-yl, unsubstituted tetrahydrofuran-3-yl; and
   in which R¹, A and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
R² represents 3-hydroxybutan-2-yl, prop-2-yn-1-yl, but-2-yn-1-yl, 2,2-dimethyl-2-methoxyethyl, methoxyethyl; or
   cyclopropylmethyl, tetrahydrofuran-3-yl, tetrahydrofuran-2-ylmethyl, tetrahydrofuran-3-ylmethyl, oxetan-3-yl, tetrahydropyran-4-yl, tetrahydro-2H-pyran-4-ylmethyl, (4-methylmorpholin-2-yl)methyl, pyridin-4-yl, pyridin-3-yl, 1,3,4-thiadiazol-2-yl, 1,3-thiazol-2-yl, piperidin-4-yl, pyrrolidin-3-yl or azetidin-3-yl which are optionally substituted,
   preferably unsubstituted cyclopropylmethyl, unsubstituted oxetan-3-yl, unsubstituted (3R)-tetrahydrofuran-3-yl, unsubstituted (3S)-tetrahydrofuran-3-yl, [(2R)-4-methylmorpholin-2-yl]methyl, [(2S)-4-methylmorpholin-2-yl]methyl, (2R,3R)-3-hydroxybutan-2-yl, (2S,3S)-3-hydroxybutan-2-yl, (2S,3R)-3-hydroxybutan-2-yl or (2R,3S)-3-hydroxybutan-2-yl; and
      in which R¹, A and R³ have the same meaning as defined in general formula (a),
      or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (I), wherein
R² represents unsubstituted tetrahydrofuran-3-yl or unsubstituted oxetan-3-yl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (I), wherein
R² represents unsubstituted (3R)-tetrahydrofuran-3-yl, (3S)-tetrahydrofuran-3-yl or unsubstituted oxetan-3-yl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (I), wherein
R² represents unsubstituted (3R)-tetrahydrofuran-3-yl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (I), wherein
R² represents [(2R)-4-methylmorpholin-2-yl]methyl, (2R,3R)-3-hydroxybutan-2-yl, or (2S,3S)-3-hydroxybutan-2-yl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (I), wherein
R² represents [(2R)-4-methylmorpholin-2-yl]methyl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (I), wherein
R² represents (2R,3R)-3-hydroxybutan-2-yl, or (2S,3S)-3-hydroxybutan-2-yl; and
   in which R¹, A and R³ have the same meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (Ia), wherein
R² represents unsubstituted tetrahydrofuran-3-yl or unsubstituted oxetan-3-yl; and
   in which R¹, A and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (Ia), wherein
R² represents unsubstituted (3R)-tetrahydrofuran-3-yl, (3S)-tetrahydrofuran-3-yl or unsubstituted oxetan-3-yl; and
   in which R¹, A and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (Ia), wherein
R² represents unsubstituted (3R)-tetrahydrofuran-3-yl; and
   in which R¹, A and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (Ia), wherein
R² represents [(2R)-4-methylmorpholin-2-yl]methyl, (2R,3R)-3-hydroxybutan-2-yl or (2S,3S)-3-hydroxybutan-2-yl; and
   in which R¹, A and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (Ia), wherein
R² represents [(2R)-4-methylmorpholin-2-yl]methyl; and
   in which R¹, A and R³ have the same meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of formula (Ia), wherein
R² represents (2R,3R)-3-hydroxybutan-2-yl, or (2S,3S)-3-hydroxybutan-2-yl; and
   in which R¹, A and R³ have the same meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl; and
   in which R² and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R¹ represents methyl or ethyl; and
   in which R² and R³ have the meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R³ represents a methyl group; and
   in which R¹ and R² have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c}, R¹ and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c}, R¹ and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c}, R¹ and R³ have the meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₃-alkyl-OR⁴, —CH₂—(C₃-C₄-cycloalkyl), C₃-C₄-cycloalkyl, —(CH₂)_{q}-(4- to 6-membered heterocycloalkyl), or —C₂-C₄-alkynyl;
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R; and
q represents an integer of 0; and
   in which R^{c}, R¹ and R³ have the meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl; and
q represents an integer of 1; and
   in which R^{c}, R¹ and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-morpholinyl, wherein the ring nitrogen atom is substituted with R^{c}; and
R^{c} represents methyl; and
q represents an integer of 1; and
   in which R¹ and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl; and
q represents an integer of 1; and
   in which R^{c}, R¹ and R³ have the meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —(CH₂)_{q}-morpholinyl, wherein the ring nitrogen atom is substituted with R^{c}; and
R^{c} represents methyl; and
q represents an integer of 1; and
   in which R¹ and R³ have the meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OH; and
   in which R¹ and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1-5 fluorine atoms, or C₁-C₂-alkoxy, optionally substituted with 1-5 fluorine atoms;
R² represents —C₂-C₄-alkyl-OH; and
   in which R¹ and R³ have the meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R³ represents methyl; and
   in which R² has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

In another preferred embodiment, the invention relates to compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R³ represents methyl; and
   in which R² has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —C₂-C₄-alkyl-OR⁴, CH₂—(C₃-C₄-cycloalkyl), C₃-C₄-cycloalkyl, —(CH₂)_{q}-(4 to 6-membered heterocycloalkyl), or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted one or two times, identically or differently, at any ring carbon atom with a substituent selected from C₁-C₄-alkyl, optionally substituted with 1-5 halogen atoms which are the same or different, a halogen atom, —NR^{a}R^{b} or —COOR⁵; and
   wherein independently any ring nitrogen atom, if present in said —(CH₂)_{q}-(4- to 6-membered heterocycloalkyl) is substituted with R^{c}; and
q represents an integer of 0; and
   in which R^{c} and R¹ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —C₂-C₃-alkyl-OR⁴, CH₂—(C₃-C₄-cycloalkyl), C₃-C₄-cycloalkyl, —(CH₂)_{q}-(4 to 6-membered heterocycloalkyl), or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted one or two times, identically or differently, at any ring carbon atom with a substituent selected from C₁-C₄-alkyl, optionally substituted with 1-5 halogen atoms which are the same or different, a halogen atom, —NR^{a}R^{b} or —COOR⁵; and
   wherein independently any ring nitrogen atom, if present in said —(CH₂)_{q}-(4- to 6-membered heterocycloalkyl) is substituted with R^{c}; and
      in which R^{c} and R¹ have the meaning as defined in general formula (I),
      or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with Rc; and wherein said —(CH₂)_{q}-(4 to 6-membered heterocycloalkyl) is preferably —(CH₂)_{q}-morpholinyl;
q represents an integer of 1; and
   in which R^{c} and R¹ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents —(CH₂)_{q}-morpholinyl, wherein the ring nitrogen atom is substituted with R^{c}; and
R^{c} represents methyl;
q represents an integer of 1; and
   in which R¹ has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R³ represents methyl;
R² represents C₂-C₄-alkyl-OH, preferably 3-hydroxybutan-2-yl; and
   in which R¹ has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₄-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c} and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using to compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₄-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c} and R³ have the meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₃-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c} and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₃-alkyl-OR⁴, —CH₂—(C₃-C₄-cycloalkyl), C₃-C₄-cycloalkyl, —(CH₂)_{q}-(4 to 6-membered heterocycloalkyl) or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
q represents an integer of 0; and
   in which R^{c} and R³ have the meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl;
q represents an integer of 1; and
   in which R^{c} and R³ have the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-morpholinyl, wherein the ring nitrogen atom is substituted with R^{c}; and
R^{c} represents methyl;
q represents an integer of 1; and
   in which R³ has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein said —(CH₂)_{q}-(4 to 6-membered heterocycloalkyl) is preferably —(CH₂)_{q}-morpholinyl;
q represents an integer of 1; and
   in which R^{c} and R³ have the meaning as defined in general formula (Ia),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-morpholinyl, wherein the ring nitrogen atom is substituted with R^{c}; and
R^{c} represents methyl;
q represents an integer of 1; and
   in which R³ has the meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using to compounds of general formula (I), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R² represents C₂-C₄-alkyl-OH, preferably 3-hydroxybutan-2-yl; and
   in which R³ has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (Ia), wherein
A represents a 6-membered heteroaryl, in particular pyrimidinyl or pyridazinyl,
   wherein said 6-membered heteroaryl is optionally substituted one or two times, identically or differently, with a substituent selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R² represents C₂-C₄-alkyl-OH, preferably 3-hydroxybutan-2-yl; and
   in which R³ has the meaning as defined in general formula (a),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —C₂-C₃-alkyl-OR⁴, CH₂—(C₃-C₄-cycloalkyl), C₃-C₄-cycloalkyl, —(CH₂)_{q}-(4- to 6-membered heterocycloalkyl), or —C₂-C₄-alkynyl,
   wherein said —CH₂—(C₃-C₄-cycloalkyl), C₃-C₄-cycloalkyl and —(CH₂)_{q}-(4- to 6-membered heterocycloalkyl) are optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and wherein independently any ring nitrogen atom, if present in said —(CH₂)_{q}-(4- to 6-membered heterocycloalkyl) is substituted with R^{c}; and
R³ represents methyl; and
q represents an integer of 0,
   in which R^{c} has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-(4- to 6-membered heterocycloalkyl); and wherein (CH₂)_{q}-(4- to 6-membered heterocycloalkyl) is optionally substituted with one or two substituents which are the same or different, at any ring carbon atom and selected from the group consisting of C₁-C₄-alkyl, optionally substituted with 1-5 halogen atoms which are the same or different, a halogen atom, —NR^{a}R^{b} and —COOR⁵; and
   wherein independently any ring nitrogen atom, if present in said —(CH₂)_{q}-(4 to 6-membered heterocycloalkyl) is substituted with R^{c}; and wherein —(CH₂)_{q}-(4- to 6-membered heterocycloalkyl) is preferably —(CH₂)_{q}-morpholinyl;
R³ represents methyl; and
q represents an integer of 1,
   in which R^{c} has the meaning as defined in general formula (I),
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents methyl or ethyl;
R² represents —(CH₂)_{q}-morpholinyl, wherein the ring nitrogen atom is substituted with R^{c}; and
R^{c} represents methyl;
R³ represents methyl; and
q represents an integer of 1,
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another embodiment of the present invention relates to a method for using compounds of general formula (I), more preferably to compounds of general formula (Ia), wherein
A represents a 6-membered heteroaryl, in particular pyrimidinyl or pyridazinyl;
   wherein said 6-membered heteroaryl is optionally substituted with one or two substituents which are the same or different, and selected from a fluorine or chlorine atom, C₁-C₂-alkyl, optionally substituted with 1 to 5 fluorine atoms, or C₁-C₂-alkoxy, optionally substituted with 1 to 5 fluorine atoms;
R¹ represents chloro;
R² represents —C₂-C₄-alkyl-OH, preferably 3-hydroxybutan-2-yl; and
R³ represents methyl,
   or an isomer, enantiomer, diastereomer, racemate, hydrate, solvate, or a salt thereof, or a mixture of same for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

The use of following compounds for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors, is disclosed, namely use of
1) 3-(cyclopropylmethoxy)-5-(5-methyl-1,3-thiazol-2-yl)-N-{(1R)-1-[2-(trifluoromethyl)pyrimidin-5-yl]ethyl}benzamide
2) 3-(cyclopropylmethoxy)-N-[(6-methylpyridazin-3-yl)methyl]-5-(5-methyl-1,3-thiazol-2-yl)benzamide
3) 3-(cyclopropylmethoxy)-N-[(5-methylpyrazin-2-yl)methyl]-5-(5-methyl-1,3-thiazol-2-yl)benzamide
4) 3-(cyclopropylmethoxy)-N-[(1R)-1-(5-methylpyrazin-2-yl)ethyl]-5-(5-methyl-1,3-thiazol-2-yl)benzamide
5) N-[1-(3-chloro-5-fluoropyridin-2-yl)ethyl]-3-(cyclopropylmethoxy)-5-(5-methyl-1,3-thiazol-2-yl)benzamide
6) N-[1-(5-chloro-3-fluoropyridin-2-yl)ethyl]-3-(cyclopropylmethoxy)-5-(5-methyl-1,3-thiazol-2-yl)benzamide
7) 3-(cyclopropylmethoxy)-N-[(1R)-1-(6-methylpyridazin-3-yl)ethyl]-5-(5-methyl-1,3-thiazol-2-yl)benzamide
8) 3-(cyclopropylmethoxy)-5-(5-methyl-1,3-thiazol-2-yl)-N-{(1R)-1-[6-(trifluoromethyl)pyridazin-3-yl]ethyl}benzamide
9) 3-(cyclopropylmethoxy)-N-[(1R)-1-(2-methylpyrimidin-5-yl)ethyl]-5-(5-methyl-1,3-thiazol-2-yl)benzamide
10) 3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide
11) N-[(5-methylpyrazin-2-yl)methyl]-3-(5-methyl-1,3-thiazol-2-yl)-5-[(3R)-tetrahydrofuran-3-yloxy]benzamide
12) N-[1-(3-chloro-5-fluoropyridin-2-yl)ethyl]-3-(5-methyl-1,3-thiazol-2-yl)-5-[(3R)-tetrahydrofuran-3-yloxy]benzamide
13) N-[1-(5-chloro-3-fluoropyridin-2-yl)ethyl]-3-(5-methyl-1,3-thiazol-2-yl)-5-[(3R)-tetrahydrofuran-3-yloxy]benzamide
14) N-[(1R)-1-(5-chloropyridin-2-yl)ethyl]-3-(5-methyl-1,3-thiazol-2-yl)-5-[(3R)-tetrahydrofuran-3-yloxy]benzamide
15) N-[(1R)-1-(5-methylpyrazin-2-yl)ethyl]-3-(5-methyl-1,3-thiazol-2-yl)-5-[(3R)-tetrahydrofuran-3-yloxy]benzamide
16) N-[(6-methylpyridazin-3-yl)methyl]-3-(5-methyl-1,3-thiazol-2-yl)-5-[(3R)-tetrahydrofuran-3-yloxy]benzamide
17) N-[(1R)-1-(6-methylpyridazin-3-yl)ethyl]-3-(5-methyl-1,3-thiazol-2-yl)-5-[(3R)-tetrahydrofuran-3-yloxy]benzamide
18) 3-(5-methyl-1,3-thiazol-2-yl)-5-[(3R)-tetrahydrofuran-3-yloxy]-N-{(1R)-1-[6-(trifluoromethyl)pyridazin-3-yl]ethyl}benzamide
19) 3-(5-methyl-1,3-thiazol-2-yl)-5-[(3R)-tetrahydrofuran-3-yloxy]-N-{[6-(trifluoromethyl)pyridazin-3-yl]methyl}benzamide
20) 3-(5-methyl-1,3-thiazol-2-yl)-5-[(3R)-tetrahydrofuran-3-yloxy]-N-{(1R)-1-[2-(trifluoromethyl)pyrimidin-5-yl]propyl}benzamide
21) N-[(1R)-1-(2-methylpyrimidin-5-yl)ethyl]-3-(5-methyl-1,3-thiazol-2-yl)-5-[(3R)-tetrahydrofuran-3-yloxy]benzamide
22) N-[(1R)-1-(6-methylpyridin-3-yl)ethyl]-3-(5-methyl-1,3-thiazol-2-yl)-5-[(3R)-tetrahydrofuran-3-yloxy]benzamide
23) N-[(1R)-1-(6-methylpyridazin-3-yl)ethyl]-3-(5-methyl-1,3-thiazol-2-yl)-5-[(3S)-tetrahydrofuran-3-yloxy]benzamide
24) 3-(5-methyl-1,3-thiazol-2-yl)-5-[(3S)-tetrahydrofuran-3-yloxy]-N-{(1R)-1-[2-(trifluoromethyl)pyrimidin-5-yl]ethyl}benzamide
25) N-[(1R)-1-(5-chloropyridin-2-yl)ethyl]-3-(5-methyl-1,3-thiazol-2-yl)-5-[(3S)-tetrahydrofuran-3-yloxy]benzamide
26) N-[(1R)-1-(5-methylpyridin-2-yl)ethyl]-3-(5-methyl-1,3-thiazol-2-yl)-5-[(3S)-tetrahydrofuran-3-yloxy]benzamide
27) N-[(1R)-1-(5-methylpyrazin-2-yl)ethyl]-3-(5-methyl-1,3-thiazol-2-yl)-5-[(3S)-tetrahydrofuran-3-yloxy]benzamide
28) N-[(1R)-1-(6-methylpyridin-3-yl)ethyl]-3-(5-methyl-1,3-thiazol-2-yl)-5-[(3S)-tetrahydrofuran-3-yloxy]benzamide
29) N-[(6-methylpyridazin-3-yl)methyl]-3-(5-methyl-1,3-thiazol-2-yl)-5-(prop-2-yn-1-yloxy)benzamide
30) N-[(5-chloro-3-fluoropyridin-2-yl)methyl]-3-(5-methyl-1,3-thiazol-2-yl)-5-(prop-2-yn-1-yloxy)benzamide
31) N-[(1R)-1-(6-methylpyridin-3-yl)ethyl]-3-(5-methyl-1,3-thiazol-2-yl)-5-(prop-2-yn-1-yloxy)benzamide
32) N-[(1R)-1-(5-methylpyrazin-2-yl)ethyl]-3-(5-methyl-1,3-thiazol-2-yl)-5-(prop-2-yn-1-yloxy)benzamide
33) N-[(5-methylpyrazin-2-yl)methyl]-3-(5-methyl-1,3-thiazol-2-yl)-5-(prop-2-yn-1-yloxy)benzamide
34) 3-(5-methyl-1,3-thiazol-2-yl)-5-(prop-2-yn-1-yloxy)-N-{(1R)-1-[2-(trifluoromethyl)pyrimidin-5-yl]ethyl}benzamide
35) N-[(1R)-1-(6-methylpyridazin-3-yl)ethyl]-3-(5-methyl-1,3-thiazol-2-yl)-5-(prop-2-yn-1-yloxy)benzamide
36) 3-(5-methyl-1,3-thiazol-2-yl)-5-(prop-2-yn-1-yloxy)-N-{(1R)-1-[6-(trifluoromethyl)pyridazin-3-yl]ethyl}benzamide
37) N-[(1R)-1-(2-methylpyrimidin-5-yl)ethyl]-3-(5-methyl-1,3-thiazol-2-yl)-5-(prop-2-yn-1-yloxy)benzamide
38) 3-(but-2-yn-1-yloxy)-5-(5-methyl-1,3-thiazol-2-yl)-N-{(1R)-1-[2-(trifluoromethyl)pyrimidin-5-yl]ethyl}benzamide
39) N-[(1R)-1-(5-methylpyrazin-2-yl)ethyl]-3-(5-methyl-1,3-thiazol-2-yl)-5-(oxetan-3-yloxy)benzamide
40) N-[(1R)-1-(2-methylpyrimidin-5-yl)ethyl]-3-(5-methyl-1,3-thiazol-2-yl)-5-(oxetan-3-yloxy)benzamide
41) N-[1-(5-chloro-3-fluoropyridin-2-yl)ethyl]-3-(5-methyl-1,3-thiazol-2-yl)-5-(oxetan-3-yloxy)benzamide
42) N-[(6-methylpyridazin-3-yl)methyl]-3-(5-methyl-1,3-thiazol-2-yl)-5-(oxetan-3-yloxy)benzamide
43) N-[(1R)-1-(5-chloropyridin-2-yl)ethyl]-3-(5-methyl-1,3-thiazol-2-yl)-5-(oxetan-3-yloxy)benzamide
44) N-[(1R)-1-(6-methylpyridazin-3-yl)ethyl]-3-(5-methyl-1,3-thiazol-2-yl)-5-(oxetan-3-yloxy)benzamide
45) 3-(5-methyl-1,3-thiazol-2-yl)-5-(oxetan-3-yloxy)-N-{(1R)-1-[2-(trifluoromethyl)pyrimidin-5-yl]ethyl}benzamide
46) N-[1-(3-chloro-5-fluoropyridin-2-yl)ethyl]-3-(5-methyl-1,3-thiazol-2-yl)-5-(oxetan-3-yloxy)benzamide
47) N-[(1R)-1-(6-methylpyridin-3-yl)ethyl]-3-(5-methyl-1,3-thiazol-2-yl)-5-(oxetan-3-yloxy)benzamide
48) 3-(5-methyl-1,3-thiazol-2-yl)-5-(oxetan-3-yloxy)-N-{[6-(trifluoromethyl)pyridazin-3-yl]methyl}benzamide
49) 3-(5-methyl-1,3-thiazol-2-yl)-5-(oxetan-3-yloxy)-N-{(1R)-1-[2-(trifluoromethyl)pyrimidin-5-yl]propyl}benzamide
50) N-[(6-methylpyridazin-3-yl)methyl]-3-(5-methyl-1,3-thiazol-2-yl)-5-[(2S)-tetrahydrofuran-2-ylmethoxy]benzamide
51) N-[(5-chloro-3-fluoropyridin-2-yl)methyl]-3-(5-methyl-1,3-thiazol-2-yl)-5-[(2S)-tetrahydrofuran-2-ylmethoxy]benzamide
52) N-[(5-methylpyrazin-2-yl)methyl]-3-(5-methyl-1,3-thiazol-2-yl)-5-[(2S)-tetrahydrofuran-2-ylmethoxy]benzamide
53) N-[(1R)-1-(5-methylpyrazin-2-yl)ethyl]-3-(5-methyl-1,3-thiazol-2-yl)-5-[(2S)-tetrahydrofuran-2-ylmethoxy]benzamide
54) 3-(5-methyl-1,3-thiazol-2-yl)-5-[(2S)-tetrahydrofuran-2-ylmethoxy]-N-{(1R)-1-[2-(trifluoromethyl)pyrimidin-5-yl]ethyl}benzamide
55) N-[(1R)-1-(6-methylpyridazin-3-yl)ethyl]-3-(5-methyl-1,3-thiazol-2-yl)-5-[(2S)-tetrahydrofuran-2-ylmethoxy]benzamide
56) 3-(5-methyl-1,3-thiazol-2-yl)-5-[(2S)-tetrahydrofuran-2-ylmethoxy]-N-{(1R)-1-[6-(trifluoromethyl)pyridazin-3-yl]ethyl}benzamide
57) N-[(1R)-1-(6-methylpyridazin-3-yl)ethyl]-3-(5-methyl-1,3-thiazol-2-yl)-5-[(2R)-tetrahydrofuran-2-ylmethoxy]benzamide
58) N-[(6-methylpyridazin-3-yl)methyl]-3-(5-methyl-1,3-thiazol-2-yl)-5-[(2R)-tetrahydrofuran-2-ylmethoxy]benzamide
59) 3-(5-methyl-1,3-thiazol-2-yl)-5-[(2R)-tetrahydrofuran-2-ylmethoxy]-N-{(1R)-1-[2-(trifluoromethyl)pyrimidin-5-yl]ethyl}benzamide
60) N-[(1R)-1-(5-methylpyrazin-2-yl)ethyl]-3-(5-methyl-1,3-thiazol-2-yl)-5-[(2R)-tetrahydrofuran-2-ylmethoxy]benzamide
61) N-[1-(5-chloro-3-fluoropyridin-2-yl)ethyl]-3-(5-methyl-1,3-thiazol-2-yl)-5-[(3S)-tetrahydrofuran-3-ylmethoxy]benzamide
62) N-[(6-methylpyridazin-3-yl)methyl]-3-(5-methyl-1,3-thiazol-2-yl)-5-[(3S)-tetrahydrofuran-3-ylmethoxy]benzamide
63) N-[(5-methylpyrazin-2-yl)methyl]-3-(5-methyl-1,3-thiazol-2-yl)-5-[(3S)-tetrahydrofuran-3-ylmethoxy]benzamide
64) N-[(1R)-1-(5-methylpyrazin-2-yl)ethyl]-3-(5-methyl-1,3-thiazol-2-yl)-5-[(3S)-tetrahydrofuran-3-ylmethoxy]benzamide
65) N-[1-(3-chloro-5-fluoropyridin-2-yl)ethyl]-3-(5-methyl-1,3-thiazol-2-yl)-5-[(3R)-tetrahydrofuran-3-ylmethoxy]benzamide
66) 3-(5-methyl-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
67) N-[(1R)-1-(6-methylpyridazin-3-yl)ethyl]-3-(5-methyl-1,3-thiazol-2-yl)-5-[(3S)-tetrahydrofuran-3-ylmethoxy]benzamide
68) 3-(5-methyl-1,3-thiazol-2-yl)-5-[(3S)-tetrahydrofuran-3-ylmethoxy]-N-{(1R)-1-[6-(trifluoromethyl)pyridazin-3-yl]ethyl}benzamide
69) 3-(5-methyl-1,3-thiazol-2-yl)-5-[(3R)-tetrahydrofuran-3-ylmethoxy]-N-{(1R)-1-[2-(trifluoromethyl)pyrimidin-5-yl]ethyl}benzamide
70) N-[(1R)-1-(5-methylpyrazin-2-yl)ethyl]-3-(5-methyl-1,3-thiazol-2-yl)-5-[(3R)-tetrahydrofuran-3-ylmethoxy]benzamide
71) N-[(6-methylpyridazin-3-yl)methyl]-3-(5-methyl-1,3-thiazol-2-yl)-5-[(3R)-tetrahydrofuran-3-ylmethoxy]benzamide
72) N-[(5-methylpyrazin-2-yl)methyl]-3-(5-methyl-1,3-thiazol-2-yl)-5-[(3R)-tetrahydrofuran-3-ylmethoxy]benzamide
73) N-[(1R)-1-(6-methylpyridazin-3-yl)ethyl]-3-(5-methyl-1,3-thiazol-2-yl)-5-[(3R)-tetrahydrofuran-3-ylmethoxy]benzamide
74) N-[1-(5-chloro-3-fluoropyridin-2-yl)ethyl]-3-(5-methyl-1,3-thiazol-2-yl)-5-[(3R)-tetrahydrofuran-3-ylmethoxy] benzamide
75) 3-(5-methyl-1,3-thiazol-2-yl)-5-[(3R)-tetrahydrofuran-3-ylmethoxy]-N-{(1R)-1-[6-(trifluoromethyl)pyridazin-3-yl]ethyl}benzamide
76) N-[1-(3-chloro-5-fluoropyridin-2-yl)ethyl]-3-(5-methyl-1,3-thiazol-2-yl)-5-(tetrahydro-2H-pyran-4-yloxy)benzamide
77) N-[1-(5-chloro-3-fluoropyridin-2-yl)ethyl]-3-(5-methyl-1,3-thiazol-2-yl)-5-(tetrahydro-2H-pyran-4-yloxy)benzamide
78) N-[(6-methylpyridazin-3-yl)methyl]-3-(5-methyl-1,3-thiazol-2-yl)-5-(tetrahydro-2H-pyran-4-yloxy)benzamide
79) N-[(5-methylpyrazin-2-yl)methyl]-3-(5-methyl-1,3-thiazol-2-yl)-5-(tetrahydro-2H-pyran-4-yloxy)benzamide
80) N-[(1R)-1-(5-methylpyrazin-2-yl)ethyl]-3-(5-methyl-1,3-thiazol-2-yl)-5-(tetrahydro-2H-pyran-4-yloxy)benzamide
81) 3-(5-methyl-1,3-thiazol-2-yl)-5-(tetrahydro-2H-pyran-4-yloxy)-N-{(1R)-1-[2-(trifluoromethyl)pyrimidin-5-yl]ethyl}benzamide
82) N-[(1R)-1-(6-methoxypyridin-3-yl)ethyl]-3-(5-methyl-1,3-thiazol-2-yl)-5-(tetrahydro-2H-pyran-4-yloxy)benzamide
83) N-[(1R)-1-(6-methylpyridazin-3-yl)ethyl]-3-(5-methyl-1,3-thiazol-2-yl)-5-(tetrahydro-2H-pyran-4-yloxy)benzamide
84) N-[(6-methoxypyridazin-3-yl)methyl]-3-(5-methyl-1,3-thiazol-2-yl)-5-(tetrahydro-2H-pyran-4-yloxy)benzamide
85) 3-(5-methyl-1,3-thiazol-2-yl)-5-(tetrahydro-2H-pyran-4-yloxy)-N-{(1R)-1-[6-(trifluoromethyl)pyridazin-3-yl]ethyl}benzamide
86) 3-(5-methyl-1,3-thiazol-2-yl)-5-(tetrahydro-2H-pyran-4-yloxy)-N-{(1R)-1-[2-(trifluoromethyl)pyrimidin-5-yl]propyl}benzamide
87) N-[(1R)-1-(6-methylpyridin-3-yl)ethyl]-3-(5-methyl-1,3-thiazol-2-yl)-5-(tetrahydro-2H-pyran-4-yloxy)benzamide
88) N-[(6-methylpyridazin-3-yl)methyl]-3-(5-methyl-1,3-thiazol-2-yl)-5-(tetrahydro-2H-pyran-4-ylmethoxy)benzamide
89) N-[(1R)-1-(5-methylpyrazin-2-yl)ethyl]-3-(5-methyl-1,3-thiazol-2-yl)-5-(tetrahydro-2H-pyran-4-ylmethoxy)benzamide
90) N-[1-(5-chloro-3-fluoropyridin-2-yl)ethyl]-3-(5-methyl-1,3-thiazol-2-yl)-5-(tetrahydro-2H-pyran-4-ylmethoxy)benzamide
91) N-[1-(3-chloro-5-fluoropyridin-2-yl)ethyl]-3-(5-methyl-1,3-thiazol-2-yl)-5-(tetrahydro-2H-pyran-4-ylmethoxy)benzamide
92) N-[(5-methylpyrazin-2-yl)methyl]-3-(5-methyl-1,3-thiazol-2-yl)-5-(tetrahydro-2H-pyran-4-ylmethoxy)benzamide
93) 3-(5-methyl-1,3-thiazol-2-yl)-5-(tetrahydro-2H-pyran-4-ylmethoxy)-N-{(1R)-1-[2-(trifluoromethyl)pyrimidin-5-yl]ethyl}benzamide
94) N-[(1R)-1-(6-methylpyridazin-3-yl)ethyl]-3-(5-methyl-1,3-thiazol-2-yl)-5-(tetrahydro-2H-pyran-4-ylmethoxy)benzamide
95) N-[(1R)-1-(6-methylpyridazin-3-yl)ethyl]-3-[(2-methylpyridin-4-yl)oxy]-5-(5-methyl-1,3-thiazol-2-yl)benzamide
96) N-[(6-methylpyridazin-3-yl)methyl]-3-[(2-methylpyridin-4-yl)oxy]-5-(5-methyl-1,3-thiazol-2-yl)benzamide
97) N-[(1R)-1-(5-methylpyrazin-2-yl)ethyl]-3-[(2-methylpyridin-4-yl)oxy]-5-(5-methyl-1,3-thiazol-2-yl)benzamide
98) 3-[(2-methylpyridin-4-yl)oxy]-5-(5-methyl-1,3-thiazol-2-yl)-N-{(1R)-1-[2-(trifluoromethyl)pyrimidin-5-yl]ethyl}benzamide
99) N-[(5-methylpyrazin-2-yl)methyl]-3-[(2-methylpyridin-4-yl)oxy]-5-(5-methyl-1,3-thiazol-2-yl)benzamide
100) 3-[(2-methylpyridin-4-yl)oxy]-5-(5-methyl-1,3-thiazol-2-yl)-N-{(1R)-1-[6-(trifluoromethyl)pyridazin-3-yl]ethyl}benzamide
101) 3-[(2-methylpyridin-4-yl)oxy]-N-[(1R)-1-(2-methylpyrimidin-5-yl)ethyl]-5-(5-methyl-1,3-thiazol-2-yl)benzamide
102) N-[(1R)-1-(6-methylpyridin-3-yl)ethyl]-3-[(2-methylpyridin-4-yl)oxy]-5-(5-methyl-1,3-thiazol-2-yl)benzamide
103) 3-[(6-methylpyridin-3-yl)oxy]-5-(5-methyl-1,3-thiazol-2-yl)-N-{(1R)-1-[2-(trifluoromethyl)pyrimidin-5-yl]ethyl}benzamide
104) N-[(1R)-1-(5-methylpyrazin-2-yl)ethyl]-3-[(5-methyl-1,3,4-thiadiazol-2-yl)oxy]-5-(5-methyl-1,3-thiazol-2-yl)benzamide
105) N-[(1R)-1-(6-methylpyridazin-3-yl)ethyl]-3-[(5-methyl-1,3,4-thiadiazol-2-yl)oxy]-5-(5-methyl-1,3-thiazol-2-yl)benzamide
106) 3-[(5-methyl-1,3,4-thiadiazol-2-yl)oxy]-5-(5-methyl-1,3-thiazol-2-yl)-N-{(1R)-1-[2-(trifluoromethyl)pyrimidin-5-yl]ethyl}benzamide
107) N-[(1R)-1-(5-methylpyrazin-2-yl)ethyl]-3-(5-methyl-1,3-thiazol-2-yl)-5-(1,3-thiazol-2-yloxy)benzamide
108) N-[(1R)-1-(6-methylpyridazin-3-yl)ethyl]-3-(5-methyl-1,3-thiazol-2-yl)-5-(1,3-thiazol-2-yloxy)benzamide
109) N-[(1R)-1-(6-methylpyridin-3-yl)ethyl]-3-(5-methyl-1,3-thiazol-2-yl)-5-(1,3-thiazol-2-yloxy)benzamide
110) N-[(1R)-1-(5-chloropyridin-2-yl)ethyl]-3-(5-chloro-1,3-thiazol-2-yl)-5-(2-methoxy-2-methylpropoxy)benzamide
111) 3-(5-chloro-1,3-thiazol-2-yl)-5-(2-methoxy-2-methylpropoxy)-N-{(1R)-1-[2-(trifluoromethyl)pyrimidin-5-yl]ethyl}benzamide
112) 3-(5-chloro-1,3-thiazol-2-yl)-5-(2-methoxy-2-methylpropoxy)-N-[(1R)-1-(5-methylpyrazin-2-yl)ethyl]benzamide
113) N-[(6-methylpyridazin-3-yl)methyl]-3-(tetrahydro-2H-pyran-4-ylmethoxy)-5-[5-(trifluoromethyl)-1,3-thiazol-2-yl]benzamide
114) 3-(5-cyclobutyl-1,3-thiazol-2-yl)-N-[(6-methylpyridazin-3-yl)methyl]-5-(tetrahydro-2H-pyran-4-ylmethoxy)benzamide
115) 3-(5-cyclobutyl-1,3-thiazol-2-yl)-5-(tetrahydro-2H-pyran-4-ylmethoxy)-N-{(1R)-1-[2-(trifluoromethyl)pyrimidin-5-yl]ethyl}benzamide
116) 3-(5-cyclobutyl-1,3-thiazol-2-yl)-N-[(1R)-1-(5-methylpyrazin-2-yl)ethyl]-5-[(3S)-tetrahydrofuran-3-ylmethoxy]benzamide
117) 3-(5-cyclobutyl-1,3-thiazol-2-yl)-N-[(6-methylpyridazin-3-yl)methyl]-5-[(3S)-tetrahydrofuran-3-ylmethoxy]benzamide
118) 3-(5-cyclobutyl-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
119) 3-(5-cyclobutyl-1,3-thiazol-2-yl)-N-[(1R)-1-(2-methylpyrimidin-5-yl)ethyl]-5-(tetrahydro-2H-pyran-4-ylmethoxy)benzamide
120) 3-(5-ethyl-1,3-thiazol-2-yl)-N-[(1R)-1-(2-methylpyrimidin-5-yl)ethyl]-5-(tetrahydro-2H-pyran-4-ylmethoxy)benzamide
121) N-[(1R)-1-(2-methylpyrimidin-5-yl)ethyl]-3-[5-(propan-2-yl)-1,3-thiazol-2-yl]-5-(tetrahydro-2H-pyran-4-ylmethoxy)benzamide
122) 3-(5-cyclobutyl-1,3-thiazol-2-yl)-N-[(1R)-1-(6-methylpyridazin-3-yl)ethyl]-5-(tetrahydro-2H-pyran-4-yloxy)benzamide
123) 3-(5-ethyl-1,3-thiazol-2-yl)-N-[(1R)-1-(6-methylpyridazin-3-yl)ethyl]-5-(tetrahydro-2H-pyran-4-yloxy)benzamide
124) 3-(5-ethyl-1,3-thiazol-2-yl)-5-(tetrahydro-2H-pyran-4-yloxy)-N-{(1R)-1-[2-(trifluoromethyl)pyrimidin-5-yl]ethyl}benzamide
125) N-[(1R)-1-(2-methylpyrimidin-5-yl)ethyl]-3-(5-methyl-1,3-thiazol-2-yl)-5-[(3S)-tetrahydrofuran-3-yloxy]benzamide
126) N-[(1R)-1-(5-methylpyrazin-2-yl)ethyl]-3-[(6-methylpyridin-3-yl)oxy]-5-(5-methyl-1,3-thiazol-2-yl)benzamide
127) N-[1-(3-chloro-5-fluoropyridin-2-yl)ethyl]-3-(5-methyl-1,3-thiazol-2-yl)-5-[(3S)-tetrahydrofuran-3-yloxy]benzamide
128) N-[(6-methylpyridazin-3-yl)methyl]-3-(5-methyl-1,3-thiazol-2-yl)-5-[(3S)-tetrahydrofuran-3-yloxy]benzamide
129) N-[1-(5-chloro-3-fluoropyridin-2-yl)ethyl]-3-(5-methyl-1,3-thiazol-2-yl)-5-[(3S)-tetrahydrofuran-3-yloxy]benzamide
130) 3-(5-cyclobutyl-1,3-thiazol-2-yl)-N-[(1R)-1-(6-methylpyridazin-3-yl)ethyl]-5-[(3S)-tetrahydrofuran-3-ylmethoxy]benzamide
131) 3-(2-methoxyethoxy)-N-[(1R)-1-(2-methylpyrimidin-5-yl)ethyl]-5-(5-methyl-1,3-thiazol-2-yl)benzamide
132) tert-butyl 4-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate
133) 3-(5-methyl-1,3-thiazol-2-yl)-5-(piperidin-4-yloxy)-N-{(1R)-1-[2-(trifluoromethyl)pyrimidin-5-yl]ethyl}benzamide
134) 3-[(1-methylpiperidin-4-yl)oxy]-5-(5-methyl-1,3-thiazol-2-yl)-N-{(1R)-1-[2-(trifluoromethyl)pyrimidin-5-yl]ethyl}benzamide
135) 3-(5-methyl-1,3-thiazol-2-yl)-5-{[1-(propan-2-yl)piperidin-4-yl]oxy}-N-{(1R)-1-[2-(trifluoromethyl)pyrimidin-5-yl]ethyl}benzamide
136) 3-{[(3R)-1-methylpyrrolidin-3-yl]oxy}-5-(5-methyl-1,3-thiazol-2-yl)-N-{(1R)-1-[2-(trifluoromethyl)pyrimidin-5-yl]ethyl}benzamide
137) 3-{[(3S)-1-methylpyrrolidin-3-yl]oxy}-5-(5-methyl-1,3-thiazol-2-yl)-N-{(1R)-1-[2-(trifluoromethyl)pyrimidin-5-yl]ethyl}benzamide
138) 3-[(1-methylazetidin-3-yl)oxy]-5-(5-methyl-1,3-thiazol-2-yl)-N-{(1R)-1-[2-(trifluoromethyl)pyrimidin-5-yl]ethyl}benzamide
139) 3-(5-methyl-1,3-thiazol-2-yl)-5-(prop-2-yn-1-yloxy)-N-{(1S)-1-[2-(trifluoromethyl)pyrimidin-5-yl]ethyl}benzamide
140) 3-(5-methyl-1,3-thiazol-2-yl)-5-(tetrahydro-2H-pyran-4-yloxy)-N-{(1S)-1-[2-(trifluoromethyl)pyrimidin-5-yl]ethyl}benzamide
141) tert-butyl 6-[3-(5-methyl-1,3-thiazol-2-yl)-5-({(1R)-1-[2-(trifluoromethyl)pyrimidin-5-yl]ethyl}carbamoyl)phenoxy]-2-azaspiro[3.3]heptane-2-carboxylate
142) 3-(5-methyl-1,3-thiazol-2-yl)-5-[(3R)-tetrahydrofuran-3-yloxy]-N-{(1R)-1-[5-(trifluoromethyl)pyrazin-2-yl]ethyl}benzamide
143) 3-(5-methyl-1,3-thiazol-2-yl)-5-(oxetan-3-yloxy)-N-{(1R)-1-[6-(trifluoromethyl)pyridin-3-yl]ethyl}benzamide

Also disclosed are the following compounds, namely:
144) 3-(1-azabicyclo[2.2.2]oct-4-yloxy)-5-(5-methyl-1,3-thiazol-2-yl)-N-{(1R)-1-[2-(trifluoromethyl)pyrimidin-5-yl]ethyl}benzamide
145) 3-[(1-acetylpiperidin-4-yl)oxy]-5-(5-methyl-1,3-thiazol-2-yl)-N-{(1R)-1-[2-(trifluoromethyl)pyrimidin-5-yl]ethyl}benzamide
146) N-{(1R)-1-[2-(difluoromethyl)pyrimidin-5-yl]ethyl}-3-(5-methyl-1,3-thiazol-2-yl)-5-[(3S)-tetrahydrofuran-3-yloxy]benzamide
147) N-{(1R)-1-[2-(difluoromethyl)pyrimidin-5-yl]ethyl}-3-(5-methyl-1,3-thiazol-2-yl)-5-(oxetan-3-yloxy)benzamide
148) N-{(1R)-1-[2-(difluoromethyl)pyrimidin-5-yl]ethyl}-3-(5-methyl-1,3-thiazol-2-yl)-5-[(3R)-tetrahydrofuran-3-yloxy]benzamide
149) N-{(1R)-1-[2-(difluoromethyl)pyrimidin-5-yl]ethyl}-3-(5-methyl-1,3-thiazol-2-yl)-5-(tetrahydro-2H-pyran-4-yloxy)benzamide
150) 3-{[(3S)-1-methylpiperidin-3-yl]oxy}-5-(5-methyl-1,3-thiazol-2-yl)-N-{(1R)-1-[2-(trifluoromethyl)pyrimidin-5-yl]ethyl}benzamide
151) 3-[(3-methyloxetan-3-yl)oxy]-5-(5-methyl-1,3-thiazol-2-yl)-N-{(1R)-1-[2-(trifluoromethyl)pyrimidin-5-yl]ethyl}benzamide
152) 3-(5-methyl-1,3-thiazol-2-yl)-5-[(3R)-tetrahydrofuran-3-yloxy]-N-{(1R)-1-[6-(trifluoromethyl)pyridin-3-yl]ethyl}benzamide
153) 3-{[(3R)-1-methylpiperidin-3-yl]oxy}-5-(5-methyl-1,3-thiazol-2-yl)-N-{(1R)-1-[2-(trifluoromethyl)pyrimidin-5-yl]ethyl}benzamide
154) 3-(5-methyl-1,3-thiazol-2-yl)-5-[2-(1H-1,2,4-triazol-1-yl)ethoxy]-N-{(1R)-1-[2-(trifluoromethyl)pyrimidin-5-yl]ethyl}benzamide
155) 3-(5-methyl-1,3-thiazol-2-yl)-5-[2-(1H-1,2,4-triazol-1-yl)ethoxy]-N-{(1R)-1-[6-(trifluoromethyl)pyridin-3-yl]ethyl}benzamide
156) 3-(5-methyl-1,3-thiazol-2-yl)-5-(oxetan-3-yloxy)-N-{(1R)-1-[6-(trifluoromethyl)pyridazin-3-yl]ethyl}benzamide
157) Trans Isomer 1; 3-{[3-hydroxybutan-2-yl]oxy}-5-(5-methyl-1,3-thiazol-2-yl)-N-{(1R)-1-[2-(trifluoromethyl)pyrimidin-5-yl]ethyl}benzamide
158) Trans Isomer 2; 3-{[3-hydroxybutan-2-yl]oxy}-5-(5-methyl-1,3-thiazol-2-yl)-N-{(1R)-1-[2-(trifluoromethyl)pyrimidin-5-yl]ethyl}benzamide
159) N-{(1R)-1-[6-(difluoromethyl)pyridin-3-yl]ethyl}-3-(5-methyl-1,3-thiazol-2-yl)-5-(oxetan-3-yloxy)benzamide
160) 3-{[trans-3-(dimethylamino)cyclobutyl]oxy}-5-(5-methyl-1,3-thiazol-2-yl)-N-{(1R)-1-[2-(trifluoromethyl)pyrimidin-5-yl]ethyl}benzamide
161) 3-(5-methyl-1,3-thiazol-2-yl)-5-[(3R)-tetrahydrofuran-3-yloxy]-N-{[2-(trifluoromethyl)pyrimidin-5-yl]methyl}benzamide
162) 3-(5-methyl-1,3-thiazol-2-yl)-5-(oxetan-3-yloxy)-N-{[2-(trifluoromethyl)pyrimidin-5-yl]methyl}benzamide
163) 3-[(3R)-1-azabicyclo[2.2.2]oct-3-yloxy]-5-(5-methyl-1,3-thiazol-2-yl)-N-{(1R)-1-[2-(trifluoromethyl)pyrimidin-5-yl]ethyl}benzamide
164) 3-(5-ethyl-1,3-thiazol-2-yl)-5-(oxetan-3-yloxy)-N-{(1R)-1-[2-(trifluoromethyl)pyrimidin-5-yl]ethyl}benzamide
165) 3-[(6-methylpyridazin-3-yl)oxy]-5-(5-methyl-1,3-thiazol-2-yl)-N-{(1R)-1-[2-(trifluoromethyl)pyrimidin-5-yl]ethyl}benzamide
166) N-{(1R)-1-[6-(difluoromethyl)pyridin-3-yl]ethyl}-3-(5-methyl-1,3-thiazol-2-yl)-5-[(3R)-tetrahydrofuran-3-yloxy]benzamide
167) 3-[(3R)-1-azabicyclo[2.2.2]oct-3-yloxy]-5-(5-methyl-1,3-thiazol-2-yl)-N-{(1R)-1-[6-(trifluoromethyl)pyridin-3-yl]ethyl}benzamide
168) 3-[(3S)-1-azabicyclo[2.2.2]oct-3-yloxy]-5-(5-ethyl-1,3-thiazol-2-yl)-N-{(1R)-1-[2-(trifluoromethyl)pyrimidin-5-yl]ethyl}benzamide
169) 3-[(3R)-1-azabicyclo[2.2.2]oct-3-yloxy]-5-(5-ethyl-1,3-thiazol-2-yl)-N-{(1R)-1-[2-(trifluoromethyl)pyrimidin-5-yl]ethyl}benzamide
170) 3-[(3S)-1-azabicyclo[2.2.2]oct-3-yloxy]-5-(5-methyl-1,3-thiazol-2-yl)-N-{(1R)-1-[6-(trifluoromethyl)pyridin-3-yl]ethyl}benzamide
171) 3-[(5-methyl-1,3,4-thiadiazol-2-yl)oxy]-5-(5-methyl-1,3-thiazol-2-yl)-N-{(1R)-1-[6-(trifluoromethyl)pyridin-3-yl]ethyl}benzamide
172) 3-[(2R)-1,4-dioxan-2-ylmethoxy]-5-(5-methyl-1,3-thiazol-2-yl)-N-{(1R)-1-[6-(trifluoromethyl)pyridin-3-yl]ethyl}benzamide
173) 3-[(2R)-1,4-dioxan-2-ylmethoxy]-5-(5-methyl-1,3-thiazol-2-yl)-N-{(1R)-1-[2-(trifluoromethyl)pyrimidin-5-yl]ethyl}benzamide
174) 3-[(2R)-1,4-dioxan-2-ylmethoxy]-5-(5-methyl-1,3-thiazol-2-yl)-N-{(1R)-1-[6-(trifluoromethyl)pyridazin-3-yl]ethyl}benzamide
175) 3-[(2S)-1,4-dioxan-2-ylmethoxy]-5-(5-methyl-1,3-thiazol-2-yl)-N-{(1R)-1-[6-(trifluoromethyl)pyridin-3-yl]ethyl}benzamide
176) 3-[(2S)-1,4-dioxan-2-ylmethoxy]-5-(5-methyl-1,3-thiazol-2-yl)-N-{(1R)-1-[2-(trifluoromethyl)pyrimidin-5-yl]ethyl}benzamide
177) 3-[(2S)-1,4-dioxan-2-ylmethoxy]-5-(5-methyl-1,3-thiazol-2-yl)-N-{(1R)-1-[6-(trifluoromethyl)pyridazin-3-yl]ethyl}benzamide
178) Trans Isomer 1; 3-{[3-hydroxybutan-2-yl]oxy}-5-(5-methyl-1,3-thiazol-2-yl)-N-{(1R)-1-[6-(trifluoromethyl)pyridazin-3-yl]ethyl}benzamide
179) Trans Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
180) Cis Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
181) Trans Isomer 1; 3-(5-chloro-1,3-thiazol-2-yl)-5-{[3-hydroxybutan-2-yl]oxy}-N-{(1R)-1-[6-(trifluoromethyl)pyridin-3-yl]ethyl}benzamide
182) Cis Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
183) Trans Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide
184) Trans Isomer 2; 3-(5-chloro-1,3-thiazol-2-yl)-5-{[3-hydroxybutan-2-yl]oxy}-N-{(1R)-1-[6-(trifluoromethyl)pyridin-3-yl]ethyl}benzamide
185) tert-Butyl (3R)-3-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate, as a mixture of diastereoisomers
186) 3-(but-2-yn-1-yloxy)-N-[(1R)-1-(6-methylpyridazin-3-yl)ethyl]-5-(5-methyl-1,3-thiazol-2-yl)benzamide
187) 3-[(3S)-1-azabicyclo[2.2.2]oct-3-yloxy]-5-(5-methyl-1,3-thiazol-2-yl)-N-{(1R)-1-[2-(trifluoromethyl)pyrimidin-5-yl]ethyl}benzamide
188) 3-(5-methyl-1,3-thiazol-2-yl)-5-(piperidin-4-yloxy)-N-{(1R)-1-[6-(trifluoromethyl)pyridazin-3-yl]ethyl}benzamide
189) 3-(2-azaspiro[3.3]hept-6-yloxy)-5-(5-methyl-1,3-thiazol-2-yl)-N-{(1R)-1-[2-(trifluoromethyl)pyrimidin-5-yl]ethyl}benzamide
190) 3-(5-methyl-1,3-thiazol-2-yl)-5-[(3S)-pyrrolidin-3-yloxy]-N-{(1R)-1-[2-(trifluoromethyl)pyrimidin-5-yl]ethyl}benzamide
191) 3-{[3-fluoropiperidin-4-yl]oxy}-5-(5-methyl-1,3-thiazol-2-yl)-N-{(1R)-1-[2-(trifluoromethyl)pyrimidin-5-yl]ethyl}benzamide, as a mixture of cis isomers
192) Diastereoisomer 1; 3-(5-methyl-1,3-thiazol-2-yl)-5-(piperidin-3-yloxy)-N-{(1R)-1-[2-(trifluoromethyl)pyrimidin-5-yl]ethyl}benzamide
193) Diastereoisomer 2; 3-(5-methyl-1,3-thiazol-2-yl)-5-(piperidin-3-yloxy)-N-{(1R)-1-[2-(trifluoromethyl)pyrimidin-5-yl]ethyl}benzamide
194) Cis Isomer 1; 3-(5-methyl-1,3-thiazol-2-yl)-5-{[2-(trifluoromethyl)piperidin-4-yl]oxy}-N-{(1R)-1-[2-(trifluoromethyl)pyrimidin-5-yl]ethyl}benzamide
195) Cis Isomer 2; 3-(5-methyl-1,3-thiazol-2-yl)-5-{[2-(trifluoromethyl)piperidin-4-yl]oxy}-N-{(1R)-1-[2-(trifluoromethyl)pyrimidin-5-yl]ethyl}benzamide
196) 3-{[2-methyl-2-azabicyclo[2.2.1]hept-5-yl]oxy}-5-(5-methyl-1,3-thiazol-2-yl)-N-{(1R)-1-[2-(trifluoromethyl)pyrimidin-5-yl]ethyl}benzamide
197) 3-[(1-methylpiperidin-4-yl)oxy]-5-(5-methyl-1,3-thiazol-2-yl)-N-{(1R)-1-[6-(trifluoromethyl)pyridazin-3-yl]ethyl}benzamide
198) 3-[(1-methylazetidin-3-yl)oxy]-5-(5-methyl-1,3-thiazol-2-yl)-N-{(1R)-1-[6-(trifluoromethyl)pyridazin-3-yl]ethyl}benzamide
199) 3-[(3-fluoro-1-methylpiperidin-4-yl)oxy]-5-(5-methyl-1,3-thiazol-2-yl)-N-{(1R)-1-[2-(trifluoromethyl)pyrimidin-5-yl]ethyl}benzamide, as a single unknown isomer
200) 3-{[1-(dimethylamino)cyclopropyl]methoxy}-5-(5-methyl-1,3-thiazol-2-yl)-N-{(1R)-1-[2-(trifluoromethyl)pyrimidin-5-yl]ethyl}benzamide
201) 3-[(2-methyl-2-azaspiro[3.3]hept-6-yl)oxy]-5-(5-methyl-1,3-thiazol-2-yl)-N-{(1R)-1-[2-(trifluoromethyl)pyrimidin-5-yl]ethyl}benzamide
202) N-{(1R)-1-[2-(difluoromethyl)pyrimidin-5-yl]ethyl}-3-[(1-methylpiperidin-4-yl)oxy]-5-(5-methyl-1,3-thiazol-2-yl)benzamide
203) 3-{[(3-endo)-8-methyl-8-azabicyclo[3.2.1]oct-3-yl]oxy}-5-(5-methyl-1,3-thiazol-2-yl)-N-{(1R)-1-[2-(trifluoromethyl)pyrimidin-5-yl]ethyl}benzamide
204) 3-{[(3-exo)-8-methyl-8-azabicyclo[3.2.1]oct-3-yl]oxy}-5-(5-methyl-1,3-thiazol-2-yl)-N-{(1R)-1-[2-(trifluoromethyl)pyrimidin-5-yl]ethyl}benzamide
205) 3-{[(4aS,7R,7aR)-4-methyloctahydrocyclopenta[b][1,4]oxazin-7-yl]oxy}-5-(5-methyl-1,3-thiazol-2-yl)-N-{(1R)-1-[2-(trifluoromethyl)pyrimidin-5-yl]ethyl}benzamide
206) 3-{[(4aS,7S,7aR)-4-methyloctahydrocyclopenta[b][1,4]oxazin-7-yl]oxy}-5-(5-methyl-1,3-thiazol-2-yl)-N-{(1R)-1-[2-(trifluoromethyl)pyrimidin-5-yl]ethyl}benzamide
207) Diastereoisomer 1; 3-[(1-methylpiperidin-3-yl)oxy]-5-(5-methyl-1,3-thiazol-2-yl)-N-{(1R)-1-[2-(trifluoromethyl)pyrimidin-5-yl]ethyl}benzamide
208) Diastereoisomer 2; 3-[(1-methylpiperidin-3-yl)oxy]-5-(5-methyl-1,3-thiazol-2-yl)-N-{(1R)-1-[2-(trifluoromethyl)pyrimidin-5-yl]ethyl}benzamide
209) Cis Isomer 1; 3-(5-methyl-1,3-thiazol-2-yl)-5-{[1-methyl-2-(trifluoromethyl)piperidin-4-yl]oxy}-N-{(1R)-1-[2-(trifluoromethyl)pyrimidin-5-yl]ethyl}benzamide
210) Cis Isomer 2; 3-(5-methyl-1,3-thiazol-2-yl)-5-{[1-methyl-2-(trifluoromethyl)piperidin-4-yl]oxy}-N-{(1R)-1-[2-(trifluoromethyl)pyrimidin-5-yl]ethyl}benzamide
211) 3-(5-methyl-1,3-thiazol-2-yl)-5-{[1-(propan-2-yl)piperidin-4-yl]oxy}-N-{(1R)-1-[6-(trifluoromethyl)pyridazin-3-yl]ethyl}benzamide
212) 3-(5-methyl-1,3-thiazol-2-yl)-5-{[(3S)-1-(propan-2-yl)pyrrolidin-3-yl]oxy}-N-{(1R)-1-[2-(trifluoromethyl)pyrimidin-5-yl]ethyl}benzamide
213) methyl 4-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate
214) ethyl 4-[3-(5-methyl-1,3-thiazol-2-yl)-5-({(1R)-1-[2-(trifluoromethyl)pyrimidin-5-yl]ethyl}carbamoyl)phenoxy]piperidine-1-carboxylate
215) ethyl (3S)-3-[3-(5-methyl-1,3-thiazol-2-yl)-5-({(1R)-1-[2-(trifluoromethyl)pyrimidin-5-yl]ethyl}carbamoyl)phenoxy]pyrrolidine-1-carboxylate
216) 3-(5-methyl-1,3-thiazol-2-yl)-5-{[1-(propan-2-yl)azetidin-3-yl]oxy}-N-{(1R)-1-[2-(trifluoromethyl)pyrimidin-5-yl]ethyl}benzamide
217) Cis Isomer 1; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide
218) Cis Isomer 2; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide
219) 3-[(1,1-dioxidotetrahydro-2H-thiopyran-4-yl)oxy]-5-(5-methyl-1,3-thiazol-2-yl)-N-{(1R)-1-[2-(trifluoromethyl)pyrimidin-5-yl]ethyl}benzamide
220) 3-[(1,1-dioxidotetrahydro-2H-thiopyran-4-yl)oxy]-5-(5-methyl-1,3-thiazol-2-yl)-N-{(1R)-1-[6-(trifluoromethyl)pyridin-3-yl]ethyl}benzamide
221) 3-(5-ethyl-1,3-thiazol-2-yl)-N-[(1R)-1-(5-methylpyrazin-2-yl)ethyl]-5-(tetrahydro-2H-pyran-4-yloxy)benzamide
222) 3-(5-ethyl-1,3-thiazol-2-yl)-N-[(6-methylpyridazin-3-yl)methyl]-5-(tetrahydro-2H-pyran-4-yloxy)benzamide
223) 3-(5-cyclobutyl-1,3-thiazol-2-yl)-N-[(1R)-1-(5-methylpyrazin-2-yl)ethyl]-5-(tetrahydro-2H-pyran-4-yloxy)benzamide
224) 3-(5-cyclobutyl-1,3-thiazol-2-yl)-N-[(6-methylpyridazin-3-yl)methyl]-5-(tetrahydro-2H-pyran-4-yloxy)benzamide
225) 3-(5-cyclobutyl-1,3-thiazol-2-yl)-5-(tetrahydro-2H-pyran-4-yloxy)-N-{(1R)-1-[2-(trifluoromethyl)pyrimidin-5-yl]ethyl}benzamide
226) 3-(5-cyclobutyl-1,3-thiazol-2-yl)-5-[(3S)-tetrahydrofuran-3-ylmethoxy]-N-{(1S)-1-[2-(trifluoromethyl)pyrimidin-5-yl]ethyl}benzamide
227) 3-(5-ethyl-1,3-thiazol-2-yl)-N-[(1R)-1-(6-methylpyridazin-3-yl)ethyl]-5-[(3R)-tetrahydrofuran-3-yloxy]benzamide
228) 3-(5-ethyl-1,3-thiazol-2-yl)-N-[(1R)-1-(5-methylpyrazin-2-yl)ethyl]-5-[(3R)-tetrahydrofuran-3-yloxy]benzamide
229) 3-(5-ethyl-1,3-thiazol-2-yl)-5-[(3R)-tetrahydrofuran-3-yloxy]-N-{(1R)-1-[2-(trifluoromethyl)pyrimidin-5-yl]ethyl}benzamide
230) 3-(5-cyclobutyl-1,3-thiazol-2-yl)-N-[(1R)-1-(6-methylpyridazin-3-yl)ethyl]-5-[(3R)-tetrahydrofuran-3-yloxy]benzamide
231) 3-(5-cyclobutyl-1,3-thiazol-2-yl)-N-[(1R)-1-(5-methylpyrazin-2-yl)ethyl]-5-[(3R)-tetrahydrofuran-3-yloxy]benzamide
232) 3-(5-cyclobutyl-1,3-thiazol-2-yl)-5-[(3R)-tetrahydrofuran-3-yloxy]-N-{(1R)-1-[2-(trifluoromethyl)pyrimidin-5-yl]ethyl}benzamide
233) N-[(1R)-1-(6-methylpyridazin-3-yl)ethyl]-3-[5-(propan-2-yl)-1,3-thiazol-2-yl]-5-[(3R)-tetrahydrofuran-3-yloxy]benzamide
234) N-[(1R)-1-(5-methylpyrazin-2-yl)ethyl]-3-[5-(propan-2-yl)-1,3-thiazol-2-yl]-5-[(3R)-tetrahydrofuran-3-yloxy]benzamide
235) 3-[5-(propan-2-yl)-1,3-thiazol-2-yl]-5-[(3R)-tetrahydrofuran-3-yloxy]-N-{(1R)-1-[2-(trifluoromethyl)pyrimidin-5-yl]ethyl}benzamide
236) 3-(5-ethyl-1,3-thiazol-2-yl)-N-[(1R)-1-(6-methylpyridazin-3-yl)ethyl]-5-[(3S)-tetrahydrofuran-3-yloxy]benzamide
237) 3-(5-ethyl-1,3-thiazol-2-yl)-N-[(1R)-1-(5-methylpyrazin-2-yl)ethyl]-5-[(3S)-tetrahydrofuran-3-yloxy]benzamide
238) 3-(5-ethyl-1,3-thiazol-2-yl)-5-[(3S)-tetrahydrofuran-3-yloxy]-N-{(1R)-1-[2-(trifluoromethyl)pyrimidin-5-yl]ethyl}benzamide
239) 3-(5-cyclobutyl-1,3-thiazol-2-yl)-N-[(1R)-1-(6-methylpyridazin-3-yl)ethyl]-5-[(3S)-tetrahydrofuran-3-yloxy]benzamide
240) 3-(5-cyclobutyl-1,3-thiazol-2-yl)-N-[(1R)-1-(5-methylpyrazin-2-yl)ethyl]-5-[(3S)-tetrahydrofuran-3-yloxy]benzamide
241) 3-(5-cyclobutyl-1,3-thiazol-2-yl)-5-[(3S)-tetrahydrofuran-3-yloxy]-N-{(1R)-1-[2-(trifluoromethyl)pyrimidin-5-yl]ethyl}benzamide
242) N-[(1R)-1-(6-methylpyridazin-3-yl)ethyl]-3-[5-(propan-2-yl)-1,3-thiazol-2-yl]-5-[(3S)-tetrahydrofuran-3-yloxy]benzamide
243) N-[(1R)-1-(5-methylpyrazin-2-yl)ethyl]-3-[5-(propan-2-yl)-1,3-thiazol-2-yl]-5-[(3S)-tetrahydrofuran-3-yloxy]benzamide
244) 3-[5-(propan-2-yl)-1,3-thiazol-2-yl]-5-[(3S)-tetrahydrofuran-3-yloxy]-N-{(1R)-1-[2-(trifluoromethyl)pyrimidin-5-yl]ethyl}benzamide
245) 3-(5-ethyl-1,3-thiazol-2-yl)-N-[(1R)-1-(6-methylpyridazin-3-yl)ethyl]-5-[(3R)-tetrahydrofuran-3-ylmethoxy]benzamide
246) 3-(5-ethyl-1,3-thiazol-2-yl)-N-[(1R)-1-(5-methylpyrazin-2-yl)ethyl]-5-[(3R)-tetrahydrofuran-3-ylmethoxy]benzamide
247) 3-(5-ethyl-1,3-thiazol-2-yl)-5-[(3R)-tetrahydrofuran-3-ylmethoxy]-N-{(1R)-1-[2-(trifluoromethyl)pyrimidin-5-yl]ethyl}benzamide
248) 3-(5-cyclobutyl-1,3-thiazol-2-yl)-N-[(1R)-1-(6-methylpyridazin-3-yl)ethyl]-5-[(3R)-tetrahydrofuran-3-ylmethoxy]benzamide
249) 3-(5-cyclobutyl-1,3-thiazol-2-yl)-N-[(1R)-1-(5-methylpyrazin-2-yl)ethyl]-5-[(3R)-tetrahydrofuran-3-ylmethoxy]benzamide
250) 3-(5-cyclobutyl-1,3-thiazol-2-yl)-5-[(3R)-tetrahydrofuran-3-ylmethoxy]-N-{(1R)-1-[2-(trifluoromethyl)pyrimidin-5-yl]ethyl}benzamide
251) N-[(1R)-1-(6-methylpyridazin-3-yl)ethyl]-3-[5-(propan-2-yl)-1,3-thiazol-2-yl]-5-[(3R)-tetrahydrofuran-3-ylmethoxy]benzamide
252) N-[(1R)-1-(5-methylpyrazin-2-yl)ethyl]-3-[5-(propan-2-yl)-1,3-thiazol-2-yl]-5-[(3R)-tetrahydrofuran-3-ylmethoxy]benzamide
253) 3-[5-(propan-2-yl)-1,3-thiazol-2-yl]-5-[(3R)-tetrahydrofuran-3-ylmethoxy]-N-{(1R)-1-[2-(trifluoromethyl)pyrimidin-5-yl]ethyl}benzamide
254) 3-(5-ethyl-1,3-thiazol-2-yl)-N-[(1R)-1-(6-methylpyridazin-3-yl)ethyl]-5-[(2R)-tetrahydrofuran-2-ylmethoxy]benzamide
255) 3-(5-ethyl-1,3-thiazol-2-yl)-N-[(1R)-1-(5-methylpyrazin-2-yl)ethyl]-5-[(2R)-tetrahydrofuran-2-ylmethoxy]benzamide
256) 3-(5-ethyl-1,3-thiazol-2-yl)-5-[(2R)-tetrahydrofuran-2-ylmethoxy]-N-{(1R)-1-[2-(trifluoromethyl)pyrimidin-5-yl]ethyl}benzamide
257) 3-(5-cyclobutyl-1,3-thiazol-2-yl)-N-[(1R)-1-(6-methylpyridazin-3-yl)ethyl]-5-[(2R)-tetrahydrofuran-2-ylmethoxy]benzamide
258) 3-(5-cyclobutyl-1,3-thiazol-2-yl)-N-[(1R)-1-(5-methylpyrazin-2-yl)ethyl]-5-[(2R)-tetrahydrofuran-2-ylmethoxy]benzamide
259) 3-(5-cyclobutyl-1,3-thiazol-2-yl)-5-[(2R)-tetrahydrofuran-2-ylmethoxy]-N-{(1R)-1-[2-(trifluoromethyl)pyrimidin-5-yl]ethyl}benzamide
260) N-[(1R)-1-(6-methylpyridazin-3-yl)ethyl]-3-[5-(propan-2-yl)-1,3-thiazol-2-yl]-5-[(2R)-tetrahydrofuran-2-ylmethoxy]benzamide
261) N-[(1R)-1-(5-methylpyrazin-2-yl)ethyl]-3-[5-(propan-2-yl)-1,3-thiazol-2-yl]-5-[(2R)-tetrahydrofuran-2-ylmethoxy]benzamide
262) 3-[5-(propan-2-yl)-1,3-thiazol-2-yl]-5-[(2R)-tetrahydrofuran-2-ylmethoxy]-N-{(1R)-1-[2-(trifluoromethyl)pyrimidin-5-yl]ethyl}benzamide
263) 3-(5-ethyl-1,3-thiazol-2-yl)-N-[(1R)-1-(6-methylpyridazin-3-yl)ethyl]-5-[(3S)-tetrahydrofuran-3-ylmethoxy]benzamide
264) 3-(5-ethyl-1,3-thiazol-2-yl)-N-[(1R)-1-(5-methylpyrazin-2-yl)ethyl]-5-[(3S)-tetrahydrofuran-3-ylmethoxy]benzamide
265) 3-(5-ethyl-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
266) N-[(1R)-1-(6-methylpyridazin-3-yl)ethyl]-3-[5-(propan-2-yl)-1,3-thiazol-2-yl]-5-[(3S)-tetrahydrofuran-3-ylmethoxy]benzamide
267) N-[(1R)-1-(5-methylpyrazin-2-yl)ethyl]-3-[5-(propan-2-yl)-1,3-thiazol-2-yl]-5-[(3S)-tetrahydrofuran-3-ylmethoxy]benzamide
268) 3-[5-(propan-2-yl)-1,3-thiazol-2-yl]-5-[(3S)-tetrahydrofuran-3-ylmethoxy]-N-{(1R)-1-[2-(trifluoromethyl)pyrimidin-5-yl]ethyl}benzamide
269) 3-(5-ethyl-1,3-thiazol-2-yl)-N-[(1R)-1-(6-methylpyridazin-3-yl)ethyl]-5-[(2S)-tetrahydrofuran-2-ylmethoxy]benzamide
270) 3-(5-ethyl-1,3-thiazol-2-yl)-N-[(1R)-1-(5-methylpyrazin-2-yl)ethyl]-5-[(2S)-tetrahydrofuran-2-ylmethoxy]benzamide
271) 3-(5-ethyl-1,3-thiazol-2-yl)-5-[(2S)-tetrahydrofuran-2-ylmethoxy]-N-{(1R)-1-[2-(trifluoromethyl)pyrimidin-5-yl]ethyl}benzamide
272) 3-(5-cyclobutyl-1,3-thiazol-2-yl)-N-[(1R)-1-(6-methylpyridazin-3-yl)ethyl]-5-[(2S)-tetrahydrofuran-2-ylmethoxy]benzamide
273) 3-(5-cyclobutyl-1,3-thiazol-2-yl)-N-[(1R)-1-(5-methylpyrazin-2-yl)ethyl]-5-[(2S)-tetrahydrofuran-2-ylmethoxy]benzamide
274) 3-(5-cyclobutyl-1,3-thiazol-2-yl)-5-[(2S)-tetrahydrofuran-2-ylmethoxy]-N-{(1R)-1-[2-(trifluoromethyl)pyrimidin-5-yl]ethyl}benzamide
275) N-[(1R)-1-(6-methylpyridazin-3-yl)ethyl]-3-[5-(propan-2-yl)-1,3-thiazol-2-yl]-5-[(2S)-tetrahydrofuran-2-ylmethoxy]benzamide
276) N-[(1R)-1-(5-methylpyrazin-2-yl)ethyl]-3-[5-(propan-2-yl)-1,3-thiazol-2-yl]-5-[(2S)-tetrahydrofuran-2-ylmethoxy]benzamide
277) 3-[5-(propan-2-yl)-1,3-thiazol-2-yl]-5-[(2S)-tetrahydrofuran-2-ylmethoxy]-N-{(1R)-1-[2-(trifluoromethyl)pyrimidin-5-yl]ethyl}benzamide
278) 3-(5-methyl-1,3-thiazol-2-yl)-5-[(3R)-tetrahydrofuran-3-yloxy]-N-{(1S)-1-[2-(trifluoromethyl)pyrimidin-5-yl]ethyl}benzamide
279) 3-(5-ethyl-1,3-thiazol-2-yl)-5-[(3R)-tetrahydrofuran-3-ylmethoxy]-N-{(1R)-1-[6-(trifluoromethyl)pyridazin-3-yl]ethyl}benzamide
280) 3-[5-(propan-2-yl)-1,3-thiazol-2-yl]-5-[(3R)-tetrahydrofuran-3-ylmethoxy]-N-{(1R)-1-[6-(trifluoromethyl)pyridazin-3-yl]ethyl}benzamide
281) 3-[5-(propan-2-yl)-1,3-thiazol-2-yl]-5-[(3R)-tetrahydrofuran-3-yloxy]-N-{(1R)-1-[6-(trifluoromethyl)pyridazin-3-yl]ethyl}benzamide
282) 3-(5-cyclobutyl-1,3-thiazol-2-yl)-5-[(3S)-tetrahydrofuran-3-yloxy]-N-{(1R)-1-[6-(trifluoromethyl)pyridazin-3-yl]ethyl}benzamide
283) 3-(5-ethyl-1,3-thiazol-2-yl)-5-[(3S)-tetrahydrofuran-3-ylmethoxy]-N-{(1R)-1-[6-(trifluoromethyl)pyridazin-3-yl]ethyl}benzamide
284) 3-(5-cyclobutyl-1,3-thiazol-2-yl)-5-[(3R)-tetrahydrofuran-3-ylmethoxy]-N-{(1R)-1-[6-(trifluoromethyl)pyridazin-3-yl]ethyl}benzamide
285) 3-[5-(propan-2-yl)-1,3-thiazol-2-yl]-5-[(3S)-tetrahydrofuran-3-ylmethoxy]-N-{(1R)-1-[6-(trifluoromethyl)pyridazin-3-yl]ethyl}benzamide
286) 3-[5-(propan-2-yl)-1,3-thiazol-2-yl]-5-[(2R)-tetrahydrofuran-2-ylmethoxy]-N-{(1R)-1-[6-(trifluoromethyl)pyridazin-3-yl]ethyl}benzamide
287) 3-(5-cyclobutyl-1,3-thiazol-2-yl)-5-[(2S)-tetrahydrofuran-2-ylmethoxy]-N-{(1R)-1-[6-(trifluoromethyl)pyridazin-3-yl]ethyl}benzamide
288) 3-[5-(propan-2-yl)-1,3-thiazol-2-yl]-5-[(2S)-tetrahydrofuran-2-ylmethoxy]-N-{(1R)-1-[6-(trifluoromethyl)pyridazin-3-yl]ethyl}benzamide
289) 3-(5-ethyl-1,3-thiazol-2-yl)-5-[(2S)-tetrahydrofuran-2-ylmethoxy]-N-{(1R)-1-[6-(trifluoromethyl)pyridazin-3-yl]ethyl}benzamide
290) 3-(5-methyl-1,3-thiazol-2-yl)-5-{[1-(2,2,2-trifluoroethyl)piperidin-4-yl]oxy}-N-{(1R)-1-[2-(trifluoromethyl)pyrimidin-5-yl]ethyl}benzamide
291) 3-{[1-(2,2-difluoroethyl)piperidin-4-yl]oxy}-5-(5-methyl-1,3-thiazol-2-yl)-N-{(1R)-1-[2-(trifluoromethyl)pyrimidin-5-yl]ethyl}benzamide
292) 3-{[1-(2,2-difluoroethyl)piperidin-4-yl]oxy}-N-[(1R)-1-(6-methylpyridazin-3-yl)ethyl]-5-(5-methyl-1,3-thiazol-2-yl)benzamide
293) 3-{[1-(2,2-difluoroethyl)piperidin-4-yl]oxy}-N-[(1R)-1-(5-methylpyrazin-2-yl)ethyl]-5-(5-methyl-1,3-thiazol-2-yl)benzamide
294) 3-{[1-(2,2-difluoroethyl)piperidin-4-yl]oxy}-N-[(6-methylpyridazin-3-yl)methyl]-5-(5-methyl-1,3-thiazol-2-yl)benzamide
295) 3-{[1-(2,2-difluoroethyl)piperidin-4-yl]oxy}-5-(5-methyl-1,3-thiazol-2-yl)-N-{(1S)-1-[2-(trifluoromethyl)pyrimidin-5-yl]ethyl}benzamide
296) 3-{[1-(2,2-difluoroethyl)piperidin-4-yl]oxy}-N-[(1R)-1-(2-methylpyrimidin-5-yl)ethyl]-5-(5-methyl-1,3-thiazol-2-yl)benzamide
297) 3-(5-methyl-1,3-thiazol-2-yl)-5-{[1-(2,2,2-trifluoroethyl)piperidin-4-yl]oxy}-N-{(1S)-1-[2-(trifluoromethyl)pyrimidin-5-yl]ethyl}benzamide
298) N-[(1R)-1-(6-methylpyridazin-3-yl)ethyl]-3-(5-methyl-1,3-thiazol-2-yl)-5-{[1-(2,2,2-trifluoroethyl)piperidin-4-yl]oxy}benzamide
299) 3-(5-methyl-1,3-thiazol-2-yl)-5-{[1-(2,2,2-trifluoroethyl)piperidin-4-yl]oxy}-N-{(1R)-1-[6-(trifluoromethyl)pyridazin-3-yl]ethyl}benzamide
300) N-[(1R)-1-(5-methylpyrazin-2-yl)ethyl]-3-(5-methyl-1,3-thiazol-2-yl)-5-{[1-(2,2,2-trifluoroethyl)piperidin-4-yl]oxy}benzamide
301) N-[(6-methylpyridazin-3-yl)methyl]-3-(5-methyl-1,3-thiazol-2-yl)-5-{[1-(2,2,2-trifluoroethyl)piperidin-4-yl]oxy}benzamide
302) N-[(1R)-1-(2-methylpyrimidin-5-yl)ethyl]-3-(5-methyl-1,3-thiazol-2-yl)-5-{[1-(2,2,2-trifluoroethyl)piperidin-4-yl]oxy}benzamide
303) 3-(5-chloro-1,3-thiazol-2-yl)-N-[(1R)-1-(6-methylpyridazin-3-yl)ethyl]-5-[(3S)-tetrahydrofuran-3-ylmethoxy]benzamide
304) 3-(5-chloro-1,3-thiazol-2-yl)-N-[(1R)-1-(6-methylpyridazin-3-yl)ethyl]-5-[(3R)-tetrahydrofuran-3-yloxy]benzamide
305) 3-(5-chloro-1,3-thiazol-2-yl)-5-[(3R)-tetrahydrofuran-3-yloxy]-N-{(1R)-1-[6-(trifluoromethyl)pyridazin-3-yl]ethyl}benzamide
306) 3-(5-chloro-1,3-thiazol-2-yl)-N-[(1R)-1-(5-methylpyrazin-2-yl)ethyl]-5-[(3R)-tetrahydrofuran-3-yloxy]benzamide
307) 3-(5-chloro-1,3-thiazol-2-yl)-5-[(3S)-tetrahydrofuran-3-yloxy]-N-{(1R)-1-[6-(trifluoromethyl)pyridazin-3-yl]ethyl}benzamide
308) 3-(5-chloro-1,3-thiazol-2-yl)-N-[(1R)-1-(5-methylpyrazin-2-yl)ethyl]-5-[(3S)-tetrahydrofuran-3-yloxy]benzamide
309) 3-(5-chloro-1,3-thiazol-2-yl)-N-[(1R)-1-(6-methylpyridazin-3-yl)ethyl]-5-[(3S)-tetrahydrofuran-3-yloxy]benzamide
310) 3-(5-chloro-1,3-thiazol-2-yl)-5-[(3S)-tetrahydrofuran-3-ylmethoxy]-N-{(1R)-1-[2-(trifluoromethyl)pyrimidin-5-yl]ethyl}benzamide
311) 3-(5-chloro-1,3-thiazol-2-yl)-N-[(1R)-1-(5-methylpyrazin-2-yl)ethyl]-5-(tetrahydro-2H-pyran-4-yloxy)benzamide
312) 3-(5-chloro-1,3-thiazol-2-yl)-N-[(1R)-1-(6-methylpyridazin-3-yl)ethyl]-5-(tetrahydro-2H-pyran-4-yloxy)benzamide
313) 3-(5-chloro-1,3-thiazol-2-yl)-5-(tetrahydro-2H-pyran-4-yloxy)-N-{(1R)-1-[6-(trifluoromethyl)pyridazin-3-yl]ethyl}benzamide
314) 3-[(3-methyloxetan-3-yl)methoxy]-5-(5-methyl-1,3-thiazol-2-yl)-N-{(1R)-1-[2-(trifluoromethyl)pyrimidin-5-yl]ethyl}benzamide
315) 3-(2-hydroxy-2-methylpropoxy)-5-(5-methyl-1,3-thiazol-2-yl)-N-{(1R)-1-[2-(trifluoromethyl)pyrimidin-5-yl]ethyl}benzamide
316) 3-[(2-methyltetrahydrofuran-2-yl)methoxy]-5-(5-methyl-1,3-thiazol-2-yl)-N-{(1R)-1-[2-(trifluoromethyl)pyrimidin-5-yl]ethyl}benzamide, as a mixture of two diastereoisomers
317) Diastereoisomer 1; 3-[(2-methyltetrahydrofuran-2-yl)methoxy]-5-(5-methyl-1,3-thiazol-2-yl)-N-{(1R)-1-[2-(trifluoromethyl)pyrimidin-5-yl]ethyl}benzamide
318) Diastereoisomer 2; 3-[(2-methyltetrahydrofuran-2-yl)methoxy]-5-(5-methyl-1,3-thiazol-2-yl)-N-{(1R)-1-[2-(trifluoromethyl)pyrimidin-5-yl]ethyl}benzamide
319) 3-[(3-methyltetrahydrofuran-3-yl)methoxy]-5-(5-methyl-1,3-thiazol-2-yl)-N-{(1R)-1-[2-(trifluoromethyl)pyrimidin-5-yl]ethyl}benzamide, as a mixture of two diastereoisomers
320) Diastereoisomer 1; 3-[(3-methyltetrahydrofuran-3-yl)methoxy]-5-(5-methyl-1,3-thiazol-2-yl)-N-{(1R)-1-[2-(trifluoromethyl)pyrimidin-5-yl]ethyl}benzamide
321) Diastereoisomer 2; 3-[(3-methyltetrahydrofuran-3-yl)methoxy]-5-(5-methyl-1,3-thiazol-2-yl)-N-{(1R)-1-[2-(trifluoromethyl)pyrimidin-5-yl]ethyl}benzamide
322) 3-[(1-methyl-6-oxopiperidin-3-yl)oxy]-5-(5-methyl-1,3-thiazol-2-yl)-N-{(1R)-1-[2-(trifluoromethyl)pyrimidin-5-yl]ethyl}benzamide, as a mixture of two diastereoisomers
323) Diastereoisomer 1; 3-[(1-methyl-6-oxopiperidin-3-yl)oxy]-5-(5-methyl-1,3-thiazol-2-yl)-N-{(1R)-1-[2-(trifluoromethyl)pyrimidin-5-yl]ethyl}benzamide
324) Diastereoisomer 2; 3-[(1-methyl-6-oxopiperidin-3-yl)oxy]-5-(5-methyl-1,3-thiazol-2-yl)-N-{(1R)-1-[2-(trifluoromethyl)pyrimidin-5-yl]ethyl}benzamide
325) 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide, as a mixture of cis isomers
326) Cis Isomer 1; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide
327) Cis Isomer 2; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide
328) 3-[(7-methyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide, as a mixture of two stereoisomers
329) Stereoisomer 1; 3-[(7-methyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide
330) Stereoisomer 2; 3-[(7-methyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide
331) 3-[(7-isopropyl-3-oxa-7-azabicyclo[3.3.1]non-9-yl)oxy]-5-(5-methyl-1,3-thiazol-2-yl)-N-{(1R)-1-[2-(trifluoromethyl)pyrimidin-5-yl]ethyl}benzamide, as a mixture of two stereoisomers
332) methyl 9-[3-(5-methyl-1,3-thiazol-2-yl)-5-({(1R)-1-[2-(trifluoromethyl)pyrimidin-5-yl]ethyl}carbamoyl)phenoxy]-3-oxa-7-azabicyclo[3.3.1]nonane-7-carboxylate, as a mixture of two stereoisomers
333) tert-butyl (2R)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
334) 3-(5-methyl-1,3-thiazol-2-yl)-5-[(2R)-morpholin-2-ylmethoxy]-N-{(1R)-1-[2-(trifluoromethyl)pyrimidin-5-yl]ethyl}benzamide
335) 3-{[(2R)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide
336) tert-butyl (2S)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
337) 3-(5-methyl-1,3-thiazol-2-yl)-5-[(2S)-morpholin-2-ylmethoxy]-N-{(1R)-1-[2-(trifluoromethyl)pyrimidin-5-yl]ethyl}benzamide
338) 3-{[(2S)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide
339) 3-(5-methyl-1,3-thiazol-2-yl)-5-[morpholin-2-ylmethoxy]-N-{(1R)-1-[2-(trifluoromethyl)pyrimidin-5-yl]ethyl}benzamide, as a mixture of diastereoisomers
340) 3-{[4-methylmorpholin-2-yl]methoxy}-5-(5-methyl-1,3-thiazol-2-yl)-N-{(1R)-1-[2-(trifluoromethyl)pyrimidin-5-yl]ethyl}benzamide, as a mixture of diastereoisomers
341) Diastereoisomer 1; 3-(fluoropiperidin-3-yl)methoxy}-5-(5-methyl-1,3-thiazol-2-yl)-N-{(1R)-1-[2-(trifluoromethyl)pyrimidin-5-yl]ethyl}benzamide
342) Diastereoisomer 2; 3-(fluoropiperidin-3-yl)methoxy}-5-(5-methyl-1,3-thiazol-2-yl)-N-{(1R)-1-[2-(trifluoromethyl)pyrimidin-5-yl]ethyl}benzamide
343) Diastereoisomer 1; 3-{[3-fluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
344) Diastereoisomer 2; 3-{[3-fluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
345) 3-[(3-fluoroazetidin-3-yl)methoxy]-5-(5-methyl-1,3-thiazol-2-yl)-N-{(1R)-1-[2-(trifluoromethyl)pyrimidin-5-yl]ethyl}benzamide
346) 3-{[4,4-difluoropiperidin-3-yl]methoxy}-5-(5-methyl-1,3-thiazol-2-yl)-N-{(1R)-1-[2-(trifluoromethyl)pyrimidin-5-yl]ethyl}benzamide, as a mixture of 2 diastereoisomers
347) 3-{[(3R)-4-methylmorpholin-3-yl]methoxy}-5-(5-methyl-1,3-thiazol-2-yl)-N-{(1R)-1-[2-(trifluoromethyl)pyrimidin-5-yl]ethyl}benzamide
348) 3-{[(3S)-4-methylmorpholin-3-yl]methoxy}-5-(5-methyl-1,3-thiazol-2-yl)-N-{(1R)-1-[6-(trifluoromethyl)pyridin-3-yl]ethyl}benzamide
349) 3-{[(3S)-4-methylmorpholin-3-yl]methoxy}-5-(5-methyl-1,3-thiazol-2-yl)-N-{(1R)-1-[2-(trifluoromethyl)pyrimidin-5-yl]ethyl}benzamide
350) 3-{[(3R)-4-methylmorpholin-3-yl]methoxy}-5-(5-methyl-1,3-thiazol-2-yl)-N-{(1R)-1-[6-(trifluoromethyl)pyridin-3-yl]ethyl}benzamide
351) 3-{[4-fluoro-1-methylpyrrolidin-2-yl]methoxy}-5-(5-methyl-1,3-thiazol-2-yl)-N-{(1R)-1-[2-(trifluoromethyl)pyrimidin-5-yl]ethyl}benzamide, as a mixture of stereoisomers
352) 3-{[4-fluoro-1-methylpyrrolidin-2-yl]methoxy}-5-(5-methyl-1,3-thiazol-2-yl)-N-{(1R)-1-[6-(trifluoromethyl)pyridin-3-yl]ethyl}benzamide, as a mixture of stereoisomers
353) 3-{[(2R)-4-methylmorpholin-2-yl]methoxy}-5-(5-methyl-1,3-thiazol-2-yl)-N-{(1R)-1-[6-(trifluoromethyl)pyridin-3-yl]ethyl}benzamide
354) 3-(5-chloro-1,3-thiazol-2-yl)-5-{[(2R)-4-methylmorpholin-2-yl]methoxy}-N-{(1R)-1-[2-(trifluoromethyl)pyrimidin-5-yl]ethyl}benzamide
355) 3-{[(2S)-4-methylmorpholin-2-yl]methoxy}-5-(5-methyl-1,3-thiazol-2-yl)-N-{[2-(trifluoromethyl)pyrimidin-5-yl]methyl}benzamide
356) N-{(1R)-1-[6-(difluoromethyl)pyridin-3-yl]ethyl}-3-{[(2R)-4-methylmorpholin-2-yl]methoxy}-5-(5-methyl-1,3-thiazol-2-yl)benzamide
357) 3-{[(2S)-4-methylmorpholin-2-yl]methoxy}-5-(5-methyl-1,3-thiazol-2-yl)-N-{(1R)-1-[6-(trifluoromethyl)pyridin-3-yl]ethyl}benzamide
358) 3-[(3-fluoro-1-methylazetidin-3-yl)methoxy]-5-(5-methyl-1,3-thiazol-2-yl)-N-{(1R)-1-[2-(trifluoromethyl)pyrimidin-5-yl]ethyl}benzamide
359) 3-{[(2R)-4-methylmorpholin-2-yl]methoxy}-5-(5-methyl-1,3-thiazol-2-yl)-N-{[2-(trifluoromethyl)pyrimidin-5-yl]methyl}benzamide
360) 3-{[(2R)-4-methylmorpholin-2-yl]methoxy}-5-(5-methyl-1,3-thiazol-2-yl)-N-{(1R)-1-[6-(trifluoromethyl)pyridazin-3-yl]ethyl}benzamide
361) 3-{[(2S)-4-methylmorpholin-2-yl]methoxy}-5-(5-methyl-1,3-thiazol-2-yl)-N-{(1R)-1-[6-(trifluoromethyl)pyridazin-3-yl]ethyl}benzamide
362) 3-(5-ethyl-1,3-thiazol-2-yl)-5-{[(2S)-4-methylmorpholin-2-yl]methoxy}-N-{(1R)-1-[2-(trifluoromethyl)pyrimidin-5-yl]ethyl}benzamide
363) 3-(5-chloro-1,3-thiazol-2-yl)-5-{[(2S)-4-methylmorpholin-2-yl]methoxy}-N-{(1R)-1-[2-(trifluoromethyl)pyrimidin-5-yl]ethyl}benzamide
364) 3-(5-ethyl-1,3-thiazol-2-yl)-5-{[(2R)-4-methylmorpholin-2-yl]methoxy}-N-{(1R)-1-[2-(trifluoromethyl)pyrimidin-5-yl]ethyl}benzamide
365) 3-{[(2S)-1-methylpyrrolidin-2-yl]methoxy}-5-(5-methyl-1,3-thiazol-2-yl)-N-{(1R)-1-[2-(trifluoromethyl)pyrimidin-5-yl]ethyl}benzamide
366) 3-{[(2R)-1-methylpyrrolidin-2-yl]methoxy}-5-(5-methyl-1,3-thiazol-2-yl)-N-{(1R)-1-[2-(trifluoromethyl)pyrimidin-5-yl]ethyl}benzamide
367) 3-[(1-methylpiperidin-4-yl)methoxy]-5-(5-methyl-1,3-thiazol-2-yl)-N-{(1R)-1-[2-(trifluoromethyl)pyrimidin-5-yl]ethyl}benzamide
368) 3-(5-methyl-1,3-thiazol-2-yl)-5-{[(2R)-4-(propan-2-yl)morpholin-2-yl]methoxy}-N-{(1R)-1-[2-(trifluoromethyl)pyrimidin-5-yl]ethyl}benzamide
369) 3-(5-methyl-1,3-thiazol-2-yl)-5-{[(2S)-4-(propan-2-yl)morpholin-2-yl]methoxy}-N-{(1R)-1-[2-(trifluoromethyl)pyrimidin-5-yl]ethyl}benzamide
370) 3-{[4,4-difluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide, as a mixture of 2 diastereoisomers
371) Diastereoisomer 1; 3-{[4,4-difluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
372) Diastereoisomer 2; 3-{[4,4-difluoro-1-methylpiperidin-3-yl]methoxy}-5-(5-methyl-1,3-thiazol-2-yl)-N-{(1R)-1-[2-(trifluoromethyl)pyrimidin-5-yl]ethyl}benzamide
373) 3-[(3-fluoro-1-methylazetidin-3-yl)methoxy]-5-(5-methyl-1,3-thiazol-2-yl)-N-{(1R)-1-[6-(trifluoromethyl)pyridin-3-yl]ethyl}benzamide
374) 3-(5-ethyl-1,3-thiazol-2-yl)-5-[(3-fluoro-1-methylazetidin-3-yl)methoxy]-N-{(1R)-1-[6-(trifluoromethyl)pyridin-3-yl]ethyl}benzamide
375) 3-{[(3R)-4-methylmorpholin-3-yl]methoxy}-5-(5-methyl-1,3-thiazol-2-yl)-N-{(1R)-1-[6-(trifluoromethyl)pyridazin-3-yl]ethyl}benzamide
376) 3-{[(3S)-4-methylmorpholin-3-yl]methoxy}-5-(5-methyl-1,3-thiazol-2-yl)-N-{(1R)-1-[6-(trifluoromethyl)pyridazin-3-yl]ethyl}benzamide
377) 3-{[(2R)-4-ethylmorpholin-2-yl]methoxy}-5-(5-methyl-1,3-thiazol-2-yl)-N-{(1R)-1-[2-(trifluoromethyl)pyrimidin-5-yl]ethyl}benzamide
378) 3-{[(2R)-4-(2,2-difluoroethyl)morpholin-2-yl]methoxy}-5-(5-methyl-1,3-thiazol-2-yl)-N-{(1R)-1-[2-(trifluoromethyl)pyrimidin-5-yl]ethyl}benzamide
379) methyl (2R)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
380) methyl (2S)-2-{[3-(5-methyl-1,3-thiazol-2-yl)-5-({(1R)-1-[2-(trifluoromethyl)pyrimidin-5-yl]ethyl}carbamoyl)phenoxy]methyl}morpholine-4-carboxylate
381) 3-(azetidin-3-ylmethoxy)-5-(5-methyl-1,3-thiazol-2-yl)-N-{(1R)-1-[2-(trifluoromethyl)pyrimidin-5-yl]ethyl}benzamide
382) 3-{[(3R)-4-methyl-5-oxomorpholin-3-yl]methoxy}-5-(5-methyl-1,3-thiazol-2-yl)-N-{(1R)-1-[2-(trifluoromethyl)pyrimidin-5-yl]ethyl}benzamide
383) 3-(5-methyl-1,3-thiazol-2-yl)-5-{[(3R)-5-oxomorpholin-3-yl]methoxy}-N-{(1R)-1-[2-(trifluoromethyl)pyrimidin-5-yl]ethyl}benzamide
384) 3-{[(5S)-3-methyl-2-oxo-1,3-oxazolidin-5-yl]methoxy}-5-(5-methyl-1,3-thiazol-2-yl)-N-{(1R)-1-[2-(trifluoromethyl)pyrimidin-5-yl]ethyl}benzamide
385) 3-{[(5R)-3-methyl-2-oxo-1,3-oxazolidin-5-yl]methoxy}-5-(5-methyl-1,3-thiazol-2-yl)-N-{(1R)-1-[2-(trifluoromethyl)pyrimidin-5-yl]ethyl}benzamide
386) 3-{[(2R)-4-methyl-5-oxomorpholin-2-yl]methoxy}-5-(5-methyl-1,3-thiazol-2-yl)-N-{(1R)-1-[2-(trifluoromethyl)pyrimidin-5-yl]ethyl}benzamide
387) 3-(5-methyl-1,3-thiazol-2-yl)-5-{[(2S)-5-oxomorpholin-2-yl]methoxy}-N-{(1R)-1-[2-(trifluoromethyl)pyrimidin-5-yl]ethyl}benzamide
388) 3-{[(2S)-4-methyl-5-oxomorpholin-2-yl]methoxy}-5-(5-methyl-1,3-thiazol-2-yl)-N-{(1R)-1-[2-(trifluoromethyl)pyrimidin-5-yl]ethyl}benzamide
389) 3-{[(3S)-4-methyl-5-oxomorpholin-3-yl]methoxy}-5-(5-methyl-1,3-thiazol-2-yl)-N-{(1R)-1-[2-(trifluoromethyl)pyrimidin-5-yl]ethyl}benzamide
390) 3-(5-methyl-1,3-thiazol-2-yl)-5-{[(3S)-5-oxomorpholin-3-yl]methoxy}-N-{(1R)-1-[2-(trifluoromethyl)pyrimidin-5-yl]ethyl}benzamide
391) tert-butyl 1-{[3-(5-methyl-1,3-thiazol-2-yl)-5-({(1R)-1-[2-(trifluoromethyl)pyrimidin-5-yl]ethyl}carbamoyl)phenoxy]methyl}-2-oxa-5-azabicyclo[2.2.1]heptane-5-carboxylate, as a mixture of 2 diastereoisomers
392) 3-[(5-isopropyl-2-oxa-5-azabicyclo[2.2.1]hept-1-yl)methoxy]-5-(5-methyl-1,3-thiazol-2-yl)-N-{(1R)-1-[2-(trifluoromethyl)pyrimidin-5-yl]ethyl}benzamide, as a mixture of 2 diastereoisomers
393) 3-[(5-methyl-2-oxa-5-azabicyclo[2.2.1]hept-1-yl)methoxy]-5-(5-methyl-1,3-thiazol-2-yl)-N-{(1R)-1-[2-(trifluoromethyl)pyrimidin-5-yl]ethyl}benzamide, as a mixture of 2 diastereoisomers
394) 3-(5-methyl-1,3-thiazol-2-yl)-5-[(1S,4S)-2-oxa-5-azabicyclo[2.2.1]hept-1-ylmethoxy]-N-{(1R)-1-[2-(trifluoromethyl)pyrimidin-5-yl]ethyl}benzamide, as a mixture of 2 diastereoisomers
395) 3-(5-methyl-1,3-thiazol-2-yl)-5-[(5-propyl-2-oxa-5-azabicyclo[2.2.1]hept-1-yl)methoxy]-N-{(1R)-1-[2-(trifluoromethyl)pyrimidin-5-yl]ethyl}benzamide, as a mixture of 2 diastereoisomers
396) methyl 1-{[3-(5-methyl-1,3-thiazol-2-yl)-5-({(1R)-1-[2-(trifluoromethyl)pyrimidin-5-yl]ethyl}carbamoyl)phenoxy]methyl}-2-oxa-5-azabicyclo[2.2.1]heptane-5-carboxylate, as a mixture of 2 diastereoisomers
397) ethyl 1-{[3-(5-methyl-1,3-thiazol-2-yl)-5-({(1R)-1-[2-(trifluoromethyl)pyrimidin-5-yl]ethyl}carbamoyl)phenoxy]methyl}-2-oxa-5-azabicyclo[2.2.1]heptane-5-carboxylate, as a mixture of 2 diastereoisomers
398) 3-{[(2S)-4-ethylmorpholin-2-yl]methoxy}-5-(5-methyl-1,3-thiazol-2-yl)-N-{(1R)-1-[2-(trifluoromethyl)pyrimidin-5-yl]ethyl}benzamide
399) tert-butyl (2R)-2-{[3-(5-methyl-1,3-thiazol-2-yl)-5-({(1S)-1-[2-(trifluoromethyl)pyrimidin-5-yl]ethyl}carbamoyl)phenoxy]methyl}morpholine-4-carboxylate
400) 3-(5-methyl-1,3-thiazol-2-yl)-5-[(2R)-morpholin-2-ylmethoxy]-N-{(1S)-1-[2-(trifluoromethyl)pyrimidin-5-yl]ethyl}benzamide
401) 3-(5-ethyl-1,3-thiazol-2-yl)-5-[(2S)-morpholin-2-ylmethoxy]-N-{(1R)-1-[6-(trifluoromethyl)pyridazin-3-yl]ethyl}benzamide
402) 3-(5-ethyl-1,3-thiazol-2-yl)-5-[(2R)-morpholin-2-ylmethoxy]-N-{(1R)-1-[6-(trifluoromethyl)pyridazin-3-yl]ethyl}benzamide
403) 3-{[(2R)-4-methylmorpholin-2-yl]methoxy}-5-(5-methyl-1,3-thiazol-2-yl)-N-{(1S)-1-[2-(trifluoromethyl)pyrimidin-5-yl]ethyl}benzamide
404) 3-{[(2S)-4-methylmorpholin-2-yl]methoxy}-5-(5-methyl-1,3-thiazol-2-yl)-N-{(1S)-1-[2-(trifluoromethyl)pyrimidin-5-yl]ethyl}benzamide
405) 3-(5-ethyl-1,3-thiazol-2-yl)-5-{[(2S)-4-methylmorpholin-2-yl]methoxy}-N-{(1R)-1-[6-(trifluoromethyl)pyridazin-3-yl]ethyl}benzamide
406) 3-(5-ethyl-1,3-thiazol-2-yl)-5-{[(2R)-4-methylmorpholin-2-yl]methoxy}-N-{(1R)-1-[6-(trifluoromethyl)pyridazin-3-yl]ethyl}benzamide.

Also disclosed is the use of following compounds for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors,
3-(5-Methyl-1,3-thiazol-2-yl)-5-[(3R)-tetrahydrofuran-3-yloxy]-N-{(1R)-1-[2-(trifluoromethyl)pyrimidin-5-yl]ethyl}benzamide;
3-(5-Methyl-1,3-thiazol-2-yl)-5-[(3S)-tetrahydrofuran-3-yloxy]-N-{(1R)-1-[2-(trifluoromethyl)pyrimidin-5-yl]ethyl}benzamide;
3-(5-Methyl-1,3-thiazol-2-yl)-5-(oxetan-3-yloxy)-N-{(1R)-1-[2-(trifluoromethyl)pyrimidin-5-yl]ethyl}benzamide;
3-(5-Ethyl-1,3-thiazol-2-yl)-5-[(3R)-tetrahydrofuran-3-yloxy]-N-{(1R)-1-[2-(trifluoromethyl)pyrimidin-5-yl]ethyl}benzamide;
3-(5-Ethyl-1,3-thiazol-2-yl)-5-[(3S)-tetrahydrofuran-3-yloxy]-N-{(1R)-1-[2-(trifluoromethyl)pyrimidin-5-yl]ethyl}benzamide;
3-(5-Ethyl-1,3-thiazol-2-yl)-5-(oxetan-3-yloxy)-N-{(1R)-1-[2-(trifluoromethyl)pyrimidin-5-yl]ethyl}benzamide;
3-(5-Methyl-1,3-thiazol-2-yl)-5-[(3R)-tetrahydrofuran-3-yloxy]-N-{(1R)-1-[6-(trifluoromethyl)pyridazin-3-yl]ethyl}benzamide;
3-(5-Methyl-1,3-thiazol-2-yl)-5-[(3S)-tetrahydrofuran-3-yloxy]-N-{(1R)-1-[6-(trifluoromethyl)pyridazin-3-yl]ethyl}benzamide.

Preferred embodiment of the present invention is the use of following compounds for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors, namely
3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide;
3-(5-methyl-1,3-thiazol-2-yl)-5-[(3S)-tetrahydrofuran-3-yloxy]-N-{(1R)-1-[2-(trifluoromethyl)pyrimidin-5-yl]ethyl}benzamide.

An even more preferred embodiment of the present invention is the use of 3-(5-methyl-1,3-thiazol-2-yl)-5-[(3R)-tetrahydrofuran-3-yloxy]-N-{(1R)-1-[2-(trifluoro-methyl)pyrimidin-5-yl]ethyl}benzamide for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another preferred embodiment of the present invention is the use of the following compounds, namely
3-(5-ethyl-1,3-thiazol-2-yl)-5-{[(2R)-4-methylmorpholin-2-yl]methoxy}-N-{(1R)-1-[2-(trifluoromethyl)pyrimidin-5-yl]ethyl}benzamide;
3-{[(2R)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide;
3-(5-ethyl-1,3-thiazol-2-yl)-5-{[(2R)-4-methylmorpholin-2-yl]methoxy}-N-{(1R)-1-[6-(trifluoromethyl)pyridazin-3-yl]ethyl}benzamide
   for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

An even more preferred embodiment of the present invention is the use of 3-{[(2R)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

Another preferred embodiment of the present invention is the use of the following compounds, namely
Trans Isomer 2; 3-{[3-hydroxybutan-2-yl]oxy}-5-(5-methyl-1,3-thiazol-2-yl)-N-{(1R)-1-[2-(trifluoromethyl)pyrimidin-5-yl]ethyl}benzamide;
Trans Isomer 1; 3-{[3-hydroxybutan-2-yl]oxy}-5-(5-methyl-1,3-thiazol-2-yl)-N-{(1R)-1-[6-(trifluoromethyl)pyridazin-3-yl]ethyl}benzamide;
Trans Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Cis Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Cis Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Trans Isomer 2; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide;
Cis Isomer 1; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide;
Cis Isomer 2; 3-[(-3-hydroxybutan-2-yl)oxy]-5-(5-methyl-1,3-thiazol-2-yl)-N-{(1R)-1-[6-(trifluoromethyl)pyridazin-3-yl]ethyl}benzamide;
Cis Isomer 1; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide;
Cis Isomer 2; 3-[(3-hydroxybutan-2-yl)oxy]-5-(5-methyl-1,3-thiazol-2-yl)-N-{(1R)-1-[2-(trifluoromethyl)pyrimidin-5-yl]ethyl}benzamide
   for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

An even more preferred embodiment of the present invention is the use of Cis Isomer 1; 3-(5-chloro-1,3-thiazol-2-yl)-5-{[3-hydroxybutan-2-yl]oxy}-N-{(1R)-1-[2-(trifluoromethyl)pyrimidin-5-yl]ethyl}benzamide for the treatment or prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

It is to be understood that the present invention relates also to the use of any combination of the preferred embodiments described above.

The synthesis of the compounds of formula (I) is described in WO2016/091776.

### Pharmaceutical Compositions of the Compounds of the Invention

This invention also relates to the use of pharmaceutical compositions containing one or more compounds of the general formula (I). These compositions can be utilised to achieve the desired pharmacological effect by administration to a patient in need thereof. A patient, for the purpose of the present invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated.

The present compounds can be administered with pharmaceutically acceptable carriers well known in the art using any effective conventional dosage unit forms, including immediate, slow and timed release preparations, orally, parenterally, topically, inhaled, nasally, sublingually, intravescially, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known in the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the present compounds may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions containing the present compounds may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The present compounds may also be administered parenterally, that is, subcutaneously, intravenously, intravescially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of the present invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxylpropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived from a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition containing the present compounds may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the present compounds in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral and intravesical administration include liposomal, polymeric microsphere and polymeric gel formulations that are known in the art.

A composition containing a compound of general formula (I) may also be administered in the form of Prolonged release, implant, or depot formulation.

Another formulation employed in the methods of the present invention employs an aerosol, which enables the delivery of the compound of the general formula (I) to the airways with minimal systemic drug exposure. There are several aerosol formulations known, which can be used for that purpose, like for example liquid or dry particles generated by nebulizers or dry powder inhalers.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions containing present compounds can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃);
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate);
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid);
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate);
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The present compounds can be administered as the sole pharmaceutical agent or in combination with one or more other pharmaceutical agents where the combination causes no unacceptable adverse effects. The present invention relates also to the use of such combinations containing the present compounds for the use in treating and/or for prophylaxis of diseases or disorders which are associated with nerve fiber sensitization, and/or other pathological conditions associated with autonomic imbalance caused by increased chemoreceptor sensitivity, in particular for the treatment of breathing disorders, Cheyne Stokes respiration, central and obstructive sleep apnea, cardiovascular disease, hypertension, resistant hypertension, and heart failure, which are related to increased activity of P2X3 receptors.

The present compounds can be combined with therapeutic agents or active ingredients, that are already approved or that are still under development for the treatment and/or prophylaxis of diseases, which are related to or mediated by P2X3 receptor.
For the treatment and/or prophylaxis of cardiovascular disease, hypertension, resistant hypertension, and heart failure, the compounds of formula (I) may be administered in combination or as co-medication with antithrombotic agents, for example and preferably from the group of platelet aggregation inhibitors, anticoagulants and profibrinolytic substances;
blood pressure lowering agents, for example and preferably from the group of calcium antagonists, angiotensin All antagonists, ACE inhibitors, vasopeptidase inhibitors, endothelin antagonists, renin inhibitors, alpha-blockers, beta-blockers, mineralocorticoid receptor antagonists such as for example eplerenone, spironolactone and finerenone and diuretics;
sympatholytic agents, for example and preferably from the group of centrally acting sympatholytic agents such as for examples moxonidine, clonidine and alpha-methyldopa.
Vasopressin receptor antagonists, such as for example and preferable Conivaptan, Tolvaptan, Lixivaptan, Mozavaptan, Satavaptan, SR-121463, RWJ 676070 or BAY 86-8050, and also the compounds described in WO 2010/105770, WO2011/104322 und WO 2016/071212,
antiarrhythmic agents, for example and preferably from the group of sodium channel blockers, beta-blockers, potassium channel blockers, calcium channel blockers, I_{f}-channels blockers, Digitalis, parasympathatolytics, sympathomimetics and vernakalant.
antidiabetic agents (hypoglycemic or antihyperglycemic agents), such as for example and preferably insulin and derivatives, sulfonylureas, biguanides, thiazolidinediones, acarbose, DPP4 inhibitors, GLP-1 analogues, or SGLT inhibitors (gliflozins).
organic nitrates and NO-donors, for example sodium nitroprusside, nitroglycerin, isosorbide mononitrate, isosorbide dinitrate, molsidomine or SIN-1, and inhalational NO;
compounds that inhibit the degradation of cyclic guanosine monophosphate (cGMP), for example inhibitors of phosphodiesterases (PDE) 1, 2, 5 and/or 9, in particular PDE-5 inhibitors such as sildenafil, vardenafil, tadalafil, udenafil, dasantafil, avanafil, mirodenafil, lodenafil or PF-00489791;
positive-inotropic agents, such as for example cardiac glycosides (digoxin, digitoxin) and beta-adrenergic and dopaminergic agonists such as isoproterenol, adrenalin, noradrenalin, dopamine or dobutamine and serelaxin
natriuretic peptides, such as for example atrial natriuretic peptide (ANP, anaritide), B-type natriuretic peptide or brain natriuretic peptide (BNP, nesiritide), C-type natriuretic peptide (CNP) or urodilatin;
calcium sensitizers, such as for example and preferably levosimendan;
NO- and heme-independent activators of soluble guanylate cyclase (sGC), such as in particular cinaciguat and also the compounds described in WO01/19355, WO01/19776, WO01/19778, WO01/19780, WO02/070462, WO02/070510; WO2013/157528, WO2015/056663, WO2009/123316, WO2016/001875, WO2016/001876, WO2016/001878, WO2000/02851, WO2012/122340, WO2013/025425, WO2014/039434, WO2016/014463, WO2009/068652, WO2009/071504, WO2010/015652, WO2010/015653, WO2015/033307, WO2016/042536, WO2009/032249, WO2010/099054, WO2012/058132, US2010/0216764, WO02/070459, WO02/070460, WO2007/045366, WO2007/045369, WO2007/045433, WO2007/045370, WO2007/045367, WO2014/012935, WO2014/012934, WO2011/141409, WO2008/119457, WO2008/119458, WO2009/127338, WO2010/102717, WO2011/051165, WO2012/076466, WO2012/139888 and WO2013/174736,
NO-independent, but heme-dependent stimulators of guanylate cyclase (sGC), such as in particular riociguat, vericiguat and also the compounds described in WO00/06568, WO00/06569, WO02/42301, WO03/095451, WO2011/147809, WO2012/004258, WO2012/028647, WO2012/059549, WO2016/081668, WO2015/187470, WO2015/088885, WO2015/088886, WO2011/149921, WO2011119518, WO2010/065275, WO2016/04445, WO2016/044447, WO2016/044446, WO2016/044441, WO2015/089182, WO2014/047111, WO2014/047325, WO2013/101830, WO2012/064559, WO2012/003405, WO2011/115804, WO2014/084312, WO2012/165399, WO03/097063, WO03/09545, WO04/009589, WO03/004503, WO2007/124854, WO2008/031513, WO2008/061657, WO2010/079120, WO2010/102717, WO2012/004259, WO2012/059548, WO2012/152630, WO2014/068099, WO2014/068104, WO2012/143510, WO2012/152629, WO2013/004785, WO2013/104598, WO2013/104597, WO2013/030288, WO2013/104703, WO2013/131923, WO2014/068095, WO2014/195333, WO2014/128109, WO2014/131760, WO2014/131741, WO2015/018808, WO2015/004105, WO2015/018814, WO98/16223, WO98/16507, WO98/23619, WO02/042299, WO02/092596, WO02/042300, WO02/042301, WO02/036120, WO02/042302, WO02/070461, WO2012/165399, WO2014/084312, WO2011115804, WO2012003405, WO2012064559, WO2014/047111, WO2014/047325, WO2011/149921, WO2010/065275 and WO2011/119518,
inhibitors of human neutrophil elastase (HNE), such as for example sivelestat or DX-890 (reltran);
compounds inhibiting the signal transduction cascade, in particular tyrosine and/or serine/threonine kinase inhibitors, such as for example nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
compounds influencing the energy metabolism of the heart, such as for example and preferably etomoxir, dichloroacetate, ranolazine or trimetazidine, bendavia/elamipritide or full or partial adenosine A1 receptor agonists as GS-9667 (previously known as CVT-3619), capadenoson and neladenoson;
compounds influencing the heart rate, such as for example and preferably ivabradine;
cardiac myosin activators, such as for example and preferably omecamtiv mecarbil (CK-1827452);
HIF-PH Inhibitors, for example and preferably Molidustat, Daprodustat, Roxadustat,
bronchodilatory agents, for example and preferably from the group of beta-adrenergic receptor agonists, such as for example Albuterol, Isoproterenol, Metaproterenol, Terbutalin, Formoterol or Salmeterol, and from the group of anticholinergic agents such as for example Ipratropiumbromide;
anti-inflammatory drugs such as non-steroidal anti-inflammatory drugs (NSAIDs) including acetylsalicylic acid (aspirin), ibuprofen and naproxen, glucocorticoids, 5-aminosalicylic acid derivativesleukotriene antagonists, alpha inhibit-TNF, ors and chemokine receptor antagonists such as CCR1, 2 and/or 5 inhibitors;
fat metabolism altering agents, for example and preferably from the group of thyroid receptor agonists, cholesterol synthesis inhibitors, such as for example and preferably HMG-CoA-reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbers, bile acid reabsorption inhibitors, lipoprotein(a) antagonists and agents that inhibit the soluble epoxidhydrolase (sEH) such as for example N,N′-Di-cyclohexylurea, 12-(3-Adamantan-1-yl-ureido)-dodecanacid or 1-Adamantan-1-yl-3-{5-[2-(2-ethoxyethoxy)ethoxy]pentyl}-urea,
Prostacyclin analogs, for example and preferable Iloprost, Beraprost, Treprostinil or Epoprostenol;
agents that mediate the remodeling of extracellular matrix, for example and preferable, matrix-metalloproteinase inhibitors such as inhibitors of MMP-1, MMP-3, MMP-8, MMP-9, MMP-10, MMP-11 and MMP-13, inhibitor of metallo-elastase (MMP-12), Chymase-Inhibitors as disclosed in WO2013/167495, Inhibitors of stromelysin, collagenases, gelatinases und aggrecanases (such as and preferable neutrophil-elastase (HNE), such as Sivelestat or DX-890;
antiepileptic agents for example and preferable of the group of classical and new antiepileptic agents, such as Carbamazepin, Diazepam/Clonazepam, Ethosuximide, Phenobarbital, Primidon, Phenytoin, Valproat, Gabapentin, Labotrigin, Levetiracetam, Oxcarbazepin, Pregabalin, Tiagabin, Topiramat, Vigabatrin.
analgetic agents for example and preferable of the group of non-opiod analgetics for example and preferable of the group of antipyretic analgetics such as ASS, paracetamol, phenacetin, metamizol, propyphenazon, phenylbutazon, and of the group of antiphlogistic analgetics such as diclofenac, indomethacin, piroxica, meloxicam and COX2 inhibitors such as celecoxib, etoricoxib, parecoxib, rofecoxib and valdecoxib, and centrally acting agents such as katadolon, and of the group of opioids such as morphin, heroin, fentanyl, alfentanil, sufentanil, remifentanil, levomethadon, pritramid, pethidin, tramadol, dihydrocodein, tilidin, nalbuphin, pentazocin, buprenorphine, of the group of 5HT1 receptor agonists such as Sumatriptan, Naratriptan, Rizatriptan, Zolmitriptan, Almotriptan, Eletriptan and Frovatriptan.

Antithrombotic agents are preferably to be understood as compounds from the group of platelet aggregation inhibitors, anticoagulants and profibrinolytic substances.

In a preferred embodiment of the invention, the compounds of formula (I) are administered in combination with a platelet aggregation inhibitor, for example and preferably aspirin, clopidogrel, ticlopidine or dipyridamole.

In a preferred embodiment of the invention, the compounds of formula (I) are administered in combination with a thrombin inhibitor, for example and preferably ximelagatran, dabigatran, melagatran, bivalirudin or enoxaparin.

In a preferred embodiment of the invention, the compounds of formula (I) are administered in combination with a GPIIb/IIIa antagonist, for example and preferably tirofiban or abciximab.

In a preferred embodiment of the invention, the compounds of formula (I) are administered in combination with a factor Xa inhibitor, for example and preferably rivaroxaban, apixaban, otamixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of formula (I) are administered in combination with a factor Xa inhibitor.

In a preferred embodiment of the invention, the compounds of formula (I) are administered in combination with heparin or a low molecular weight (LMW) heparin derivative.

In a preferred embodiment of the invention, the compounds of formula (I) are administered in combination with a vitamin K antagonist, for example and preferably coumarin or warfarin.

Blood pressure lowering agents are preferably to be understood as compounds from the group of calcium antagonists, angiotensin All antagonists, ACE inhibitors, vasopeptidase inhibitors, endothelin antagonists, renin inhibitors, alpha-blockers, centrally acting sympatholytics beta-blockers, mineralocorticoid receptor antagonists and diuretics.

In a preferred embodiment of the invention, the compounds of formula (I) are administered in combination with a calcium antagonist, for example and preferably nifedipine, amlodipine, verapamil or diltiazem.

In a preferred embodiment of the invention, the compounds of formula (I) are administered in combination with an alpha-1-receptor blocker, for example and preferably prazosin, tamsulosin, bunazosin, doxazosin, phenoxybenzamin, terazosin or urapidil,

In a preferred embodiment of the invention, the compounds of formula (I) are administered in combination with a centrally acting sympatholytic agent, for example and preferably alpha-methydopa, moxonidine or clonidine.

In a preferred embodiment of the invention, the compounds of formula (I) are administered in combination with a beta-blocker, for example and preferably propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of formula (I) are administered in combination with an angiotensin All receptor antagonist, for example and preferably losartan, candesartan, valsartan, telmisartan, irbesartan, olmesartan, eprosartan or azilsartan.

In a preferred embodiment of the invention, the compounds of formula (I) are administered in combination with a vasopeptidase inhibitor or inhibitor of neutral endopeptidase (NEP), such as for example and preferably sacubitril, omapatrilat or AVE-7688.

In a preferred embodiment of the invention, the compounds of formula (I) are administered in combination with a dual angiotensin All receptor antagonist/NEP inhibitor (ARNI), for example and preferably LCZ696 (Entresto).

In a preferred embodiment of the invention, the compounds of formula (I) are administered in combination with an ACE inhibitor, for example and preferably enalapril, captopril, lisinopril, ramipril, delapril, fosinopril, quinopril, perindopril or trandopril.

In a preferred embodiment of the invention, the compounds of formula (I) are administered in combination with an endothelin antagonist, for example and preferably bosentan, darusentan, ambrisentan, tezosentan, sitaxsentan or atrasentan.

In a preferred embodiment of the invention, the compounds of formula (I) are administered in combination with a renin inhibitor, for example and preferably aliskiren, SPP-600 or SPP-800.

In a preferred embodiment of the invention, the compounds of formula (I) are administered in combination with a mineralocorticoid receptor antagonist, for example and preferably finerenone, spironolactone, canrenone, potassium canrenoate, eplerenone, CS-3150, or MT-3995.

In a preferred embodiment of the invention, the compounds of formula (I) are administered in combination with a diuretic, such as for example and preferably furosemide, bumetanide, piretanide, torsemide, bendroflumethiazide, chlorothiazide, hydrochlorothiazide, xipamide, indapamide, hydroflumethiazide, methyclothiazide, polythiazide, trichloromethiazide, chlorothalidone, metolazone, quinethazone, acetazolamide, dichlorophenamide, methazolamide, glycerine, isosorbide, mannitol, amiloride or triamterene.

sGC modulating agents are preferably to be understood as compounds from the group of NO- and heme-independent activators of soluble guanylate cyclase (sGC) and NO-independent, but heme-dependent stimulators of guanylate cyclase (sGC).

In a preferred embodiment of the invention, the compounds of formula (I) are administered in combination with a NO- and heme-independent activators of soluble guanylate cyclase (sGC), such as in particular cinaciguat.

In a preferred embodiment of the invention, the compounds of formula (I) are administered in combination with a NO-independent, but heme-dependent stimulators of guanylate cyclase (sGC), such as in particular riociguat, vericiguat.

Antiarrhythmic agents are preferably to be understood as compounds from the group of sodium channel blockers, beta-blockers, potassium channel blockers, calcium channel blockers, If-channels blockers, Digitalis, parasympathatolytics, sympathomimetics and vernakalant.

In a preferred embodiment of the invention, the compounds of formula (I) are administered in combination with a sodium channel blocker (class I antiarrhythmic agent) such as Chinidin, Ajmalin, Prajmalin, Disopyramid, Lidocain, Mexiletin, Tocainind, Phenytoin, Aprinidin, Propafenon, Flecainid, Lorcainid.

In a preferred embodiment of the invention, the compounds of formula (I) are administered in combination with a beta receptor blocker (class II antiarrhythmic agent) such as Acebutulol, Atenolol, Betaxolol, Bisoprolol, Metoprolol, Oxprenolol, Pindolol, Propanolol, Sotalol, Timolol.

In a preferred embodiment of the invention, the compounds of formula (I) are administered in combination with a potassium channel blocker (class III antiarrhythmic agent) such as Sotalol, Amiodaron, Dronedaron.

In a preferred embodiment of the invention, the compounds of formula (I) are administered in combination with a potassium channel blocker (class IV antiarrhythmic agent) such as Diltiazem, Gallopramil, Verapamil.

In a preferred embodiment of the invention, the compounds of formula (I) are administered in combination with Digoxin or Vernakalant.

Antiepileptic agents are preferably to be understood as compounds from the group of classic and new antiepileptic agents.

In a preferred embodiment of the invention, the compounds of formula (I) are administered in combination with classic antiepileptic agents such as Carbamazepin, Diazepam/Clonazepam, Ethosuximide, Phenobarbital, Primidon, Phenytoin, Valproat.

In a preferred embodiment of the invention, the compounds of formula (I) are administered in combination with new antiepileptic agents such as Gabapentin, Labotrigin, Levetiracetam, Oxcarbazepin, Pregabalin, Tiagabin, Topiramat, Vigabatrin.

Analgetic agents are preferably to be understood as compounds from the group of non-opiod analgetics, antiphlogistic analgetics, COX2 inhibitors, centrally acting analgetics, opioids and 5HT1 Receptor Agonists.

In a preferred embodiment of the invention, the compounds of formula (I) are administered in combination with non-opiod analgetics such as ASS, paracetamol, phenacetin, metamizol, propyphenazon, phenylbutazon.

In a preferred embodiment of the invention, the compounds of formula (I) are administered in combination with antiphlogistic analgetics such as diclofenac, indomethacin, piroxica, meloxicam.

In a preferred embodiment of the invention, the compounds of formula (I) are administered in combination with COX2 inhibitors such as celecoxib, etoricoxib, parecoxib, rofecoxib and valdecoxib.

In a preferred embodiment of the invention, the compounds of formula (I) re administered in combination with centrally acting agents such as katadolon.

In a preferred embodiment of the invention, the compounds of formula (I) administered in combination with opioids such as morphin, heroin, fentanyl, alfentanil, sufentanil, remifentanil, levomethadon, pritramid, pethidin, tramadol, dihydrocodein, tilidin, nalbuphin, pentazocin, buprenorphine.

In a preferred embodiment of the invention, the compounds of formula (I) are administered in combination with receptor agonists such as Sumatriptan, Naratriptan, Rizatriptan, Zolmitriptan, Almotriptan, Eletriptan and Frovatriptan.

Blood hematocrit increasing agents are preferably to be understood as compounds from the group of HIF-PH Inhibitors.

In a preferred embodiment of the invention, the compounds of formula (I) are administered in combination with HIF-PH Inhibitors such as Molidustat, Daprodustat, Roxadustat. Fat metabolism altering agents are preferably to be understood as compounds from the group of CETP inhibitors, thyroid receptor agonists, cholesterol synthesis inhibitors such as HMG-CoA-reductase or squalene synthesis inhibitors, ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and lipoprotein(a) antagonists.

In a preferred embodiment of the invention, the compounds of formula (I) are administered in combination with a CETP inhibitor, for example and preferably dalcetrapib, anacetrapib, BAY 60-5521 or CETP-vaccine (Avant).

In a preferred embodiment of the invention, the compounds of formula (I) are administered in combination with a thyroid receptor agonist, for example and preferably D-thyroxin, 3,5,3′-triiodothyronin (T3), CGS 23425 or axitirome (CGS 26214).

In a preferred embodiment of the invention, the compounds of formula (I) are administered in combination with an HMG-CoA-reductase inhibitor from the class of statins, for example and preferably lovastatin, simvastatin, pravastatin, fluvastatin, atorvastatin, rosuvastatin or pitavastatin.

In a preferred embodiment of the invention, the compounds of formula (I) administered in combination with a squalene synthesis inhibitor, for example and preferably BMS-188494 or TAK-475.

In a preferred embodiment of the invention, the compounds of formula (I) are administered in combination with an ACAT inhibitor, for example and preferably avasimibe, melinamide, pactimibe, eflucimibe or SMP-797.

In a preferred embodiment of the invention, the compounds of formula (I) are administered in combination with an MTP inhibitor, for example and preferably implitapide, R-103757, BMS-201038 or JTT-130.

In a preferred embodiment of the invention, the compounds of formula (I) are administered in combination with a PPAR-gamma agonist, for example and preferably pioglitazone or rosiglitazone.

In a preferred embodiment of the invention, the compounds of formula (I) are administered in combination with a PPAR-delta agonist, for example and preferably GW 501516 or BAY 68-5042.

In a preferred embodiment of the invention, the compounds of formula (I) are administered in combination with a cholesterol absorption inhibitor, for example and preferably ezetimibe, tiqueside or pamaqueside.

In a preferred embodiment of the invention, the compounds of formula (I) are administered in combination with a lipase inhibitor, for example and preferably orlistat.

In a preferred embodiment of the invention, the compounds of formula (I) are administered in combination with a polymeric bile acid adsorber, for example and preferably cholestyramine, colestipol, colesolvam, CholestaGel or colestimide.

In a preferred embodiment of the invention, the compounds of formula (I) are administered in combination with a bile acid reabsorption inhibitor, for example and preferably ASBT (=IBAT) inhibitors such as AZD-7806, S-8921, AK-105, BARI-1741, SC-435 or SC-635.

In a preferred embodiment of the invention, the compounds of formula (I) e administered in combination with a lipoprotein(a) antagonist, for example and preferably gemcabene calcium (CI-1027) or nicotinic acid.

In a preferred embodiment of the invention, the compounds of formula (I) are administered in combination with antidiabetics (hypoglycemic or antihyperglycemic agents), such as for example and preferably insulin and derivatives, sulfonylureas such as tolbutamide, carbutamide, acetohexamide, chlorpropamide, glipizide, gliclazide, glibenclamide, glyburide, glibornuride, gliquidone, glisoxepide, glyclopyramide, glimepiride, JB253 and JB558, meglitinides such as repaglinide and nateglinide, biguanides such as metformin and buformin, thiazolidinediones such as rosiglitazone and pioglitazone, alpha-glucosidase inhibitors such as miglitol, acarbose and voglibose, DPP4 inhibitors such as vildagliptin, sitagliptin, saxagliptin, linagliptin, alogliptin, septagliptin and teneligliptin, GLP-1 analogues such as exenatide (also exendin-4, liraglutide, lixisenatide and taspoglutide, or SGLT inhibitors (gliflozins) such as canagliflozin, dapagliflozin and empagliflozin.

In a particularly preferred embodiment, the compounds of formula (I) are administered in combination with one or more additional therapeutic agents selected from the group consisting of diuretics, angiotensin All antagonists, ACE inhibitors, beta-receptor blockers, mineralocorticoid receptor antagonists, antidiabetics, organic nitrates and NO donors, activators and stimulators of the soluble guanylate cyclase (sGC), and positive-inotropic agents.

Thus, in a further embodiment, the present invention relates to pharmaceutical compositions comprising at least one of the compounds of formula (I) according to the invention and one or more additional therapeutic agents for the treatment and/or prevention of diseases, especially of the aforementioned diseases.

### Methods of Treating

The present invention relates to a method for using the present compounds and compositions thereof, to inhibit the P2X3 receptor and therefore achieve an efficacious treatment of breathing disorders, Cheyne Stokes respiration, central and obstructive sleep apnea, cardiovascular disease, hypertension, resistant hypertension, and heart failure. The present invention also provides a method for using the compounds of formula (I) and compositions thereof, to selectively inhibit the P2X3 receptor over the P2X2/3 receptor which means at least 10-fold selectivity over the P2X2/3 receptor. The advantage of having such selective compounds results in a is the access to a method of treating breathing disorders, Cheyne Stokes respiration, central and obstructive sleep apnea, cardiovascular disease, hypertension, resistant hypertension, and heart failure with less or no effect to the taste sensitivity of the patient in need of a chronic treatment of breathing disorders, Cheyne Stokes respiration, central and obstructive sleep apnea, cardiovascular disease, hypertension, resistant hypertension, and heart failure. This provides the advantage of having an effective treatment for breathing disorders, Cheyne Stokes respiration, central and obstructive sleep apnea, cardiovascular disease, hypertension, resistant hypertension, and heart failure with less or no effect to the taste sensitivity of the patient in need of a treatment of breathing disorders, Cheyne Stokes respiration, central and obstructive sleep apnea, cardiovascular disease, hypertension, resistant hypertension, and heart failure available which may be used as chronic treatment if necessary.

Therefore, the quality of life of patients with breathing disorders, Cheyne Stokes respiration, central and obstructive sleep apnea, cardiovascular disease, hypertension, resistant hypertension, and heart failure with less or no effect to the taste sensitivity of the patient in need of a treatment of breathing disorders, Cheyne Stokes respiration, central and obstructive sleep apnea, cardiovascular disease, hypertension, resistant hypertension, and heart failure may be highly improved.

The compounds of formula (I) are also useful in a method for using the present compounds and compositions thereof, to selectively inhibit the P2X3 receptor over the P2X2/3 receptor with at least 10-fold selectivity over the P2X2/3 receptor. In addition to that, the present invention also provides a method of treating mammalian including human disorders and diseases, i.e. breathing disorders, Cheyne Stokes respiration, central and obstructive sleep apnea, cardiovascular disease, hypertension, resistant hypertension, and heart failure may be highly improved.

The present invention relates to a method for using the present compounds and compositions thereof, to treat mammalian, including human, disorders and diseases which include but are not limited to:
cardiovascular diseases including but not limited to acute and chronic heart failure in¬cluding worsening chronic heart failure (or hospitalization for heart failure) and congestive heart failure, heart failure with reduced ejection fraction, systolic heart failure, heart failure with preserved ejection fraction, diastolic heart failure, heart failure with normal ejection fraction, post-myocardial infarction heart failure, right heart failure, left heart failure, ischemic cardiomyopathy, dilatative cardiomyopathy, hypertrophic cardiomyopathy, valvular heart failure, diabetic cardiomyopathy, augmented chemoreflex, autonomic imbalance, arterial hypertension, resistant hypertension, arterial pul¬mo¬nary hypertension, coronary heart disease, stable and unstable angina pectoris, acute coronary syndrome (ACS), acute myocardial infarction, STEMI, NSTEMI, disturbances of atrial and ventricular rhythm and conduction disturbances, for example atrioventricular blocks of degree I-III (AVB I-III), supraventricular tachyarrhythmia, atrial fibrillation, paroxysmal atrial fibrillation, persistant atrial fibrillation, permanent atrial fibrillation, atrial flutter, sinus arrhythmia, ven¬tricular fibrillation, ventricular flutter, ventricular tachy¬arrhyth¬mia, torsade-de-pointes tachy¬cardia, atrial and ventricular extrasystoles, AV-junction extrasystoles, sick-sinus syndrome, syncopes, cardiac death, AV-node re-entry tachycardia and Wolff-Parkinson-White syn¬drome, autoimmune heart diseases, pericarditis, endocarditis, valvu¬litis, aortitis, cardiomyo¬pathies, myocarditis, shock such as cardiogenic shock, septic shock and ana¬phy¬lactic shock, aneurysms, Boxer cardiomyopathy (premature ventricular contraction), further¬more thromboembolic diseases and ischaemias such as peripheral perfusion disturbances, reperfusion injury, arterial and venous throm¬boses, myocardial insufficiency, endothelial dys¬function, micro- and macrovascular damage (vasculitis) and for preventing restenoses such as after thrombolysis therapies, percutaneous trans¬luminal angioplasty (PTA), percutaneous transluminal coronary angio¬plasty (PTCA), heart trans¬plan¬tation and bypass opera¬tions, arterio¬sclerosis, disturbances of lipid metabolism, hypo¬lipo¬protein¬aemias, dyslipidemias, hypertriglyceridemias, hyperlipid¬emias and combined hyperlipid-emias, hyper¬cholesterolaemias, abetalipoproteinaemia, sitosterol¬emia, xantho-matosis, Tangier disease, adipositas, obesity, metabolic syndrome, diabetes, insulin resistance, transi¬tory ischemic attacks, stroke, inflam¬ma¬tory cardiovascular diseases, myocarditis, peripheral and cardiac vascular diseases, peri¬pheral circulation disorders, peripheral artery disease, spas¬ms of the coronary arteries and peripheral arteries, and edema such as, for example, pulmonary edema, cerebral edema, renal edema and heart failure-related edema, periodic breathing disorders, central sleep apnea, obstructive sleep apnea, combined central and obstructive sleep apnea, Cheyne-Stokes respiration, and Chaga's disease.

An embodiment of the present invention relates to a method for using the compounds of formula (I) and compositions thereof, to treat breathing disorders, Cheyne Stokes respiration, central and obstructive sleep apnea, cardiovascular disease, hypertension, resistant hypertension, and heart failure.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving a condition, disease or disorder such as a gynaecological disease, urinary tract disease, respiratory disorder or arthritis.

In the sense of the present invention, the term heart failure also includes more specific or related disease forms such as right heart failure, left heart failure, global insufficiency, ischemic cardio¬myopathy, dilatative cardiomyopathy, congenital heart defects, heart valve defects, heart failure with heart valve defects, mitral valve stenosis, mitral valve insufficiency, aortic valve stenosis, aortic valve insufficiency, tricuspidal stenosis, tricuspidal¬insufficiency, pulmonary valve stenosis, pulmonary valve insufficiency, combined heart valve defects, heart muscle inflammation (myo¬carditis), chronic myocarditis, acute myocarditis, viral myocarditis, diabetic heart failure, alcohol-toxic cardiomyopathy, cardiac storage diseases, heart failure with preserved ejection fraction (HFpEF or dia¬stolic heart failure), and heart failure with reduced ejection fraction (HFrEF or systolic heart failure) and heart failure with normal ejection fraction (HFnEF).

### Dose and Administration

Based upon standard laboratory techniques known to evaluate compounds useful for the treatment of disorders and/or disease which are mediated by the P2X3 receptor, by standard toxicity tests and by standard pharmacological assays for the determination of treatment of the conditions identified above in mammals, and by comparison of these results with the results of known medicaments that are used to treat these conditions, the effective dosage of the present compounds can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, preferably from about 0.01 mg/kg to about 20 mg/kg body weight per day. A preferred administration of present compounds includes but is not limited to 0.1 mg/kg to about 10 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. A preferred oral unit dosage for an administration of the present compounds includes but is not limited to 0.1 mg/kg to about 10 mg/kg body weight one to three times a day to once a week. The average daily dosage for administration by injection, including intravenous, intravesical, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily.

The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of the present compounds or a pharmaceutically acceptable salt or ester or composition thereof can be ascertained by those skilled in the art using conventional treatment tests.

Methods of testing for a particular pharmacological or pharmaceutical property are well known to persons skilled in the art.

The example testing experiments described herein serve to illustrate the present invention and the invention is not limited to the examples given.

### Biological Assays

Examples were tested in selected biological assays one or more times. Unless stated otherwise, when tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

Data of compounds of formula (I) obtained from intracellular calcium measurement to assess antagonist activity at human P2X3 and human P2X2/3 receptors are described in WO2016/091776.

### In Vivo Assay for Ventilatory Response Under Anesthesia

The aim of this study is to test the effect of P2X3 antagonism on chemoreflex sensitivity in anesthetized animals. Reduction in breathing rate increase in response to hypoxic challenge is interpreted as a reduction in chemoreflex sensitivity.

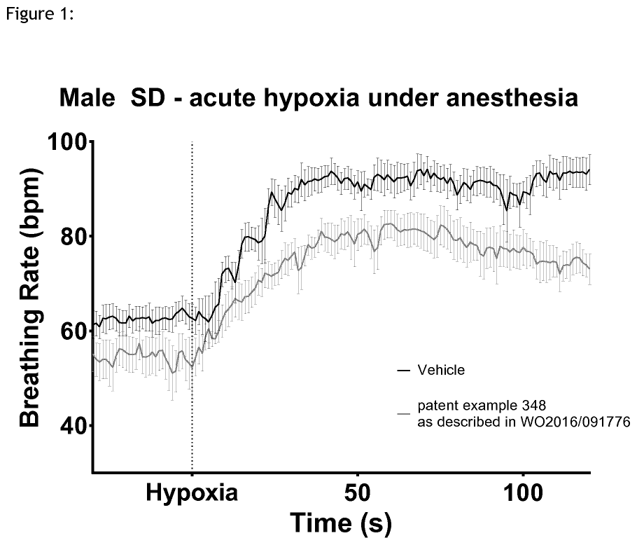

FIG. 1 illustrates the breathing rate response of anesthetized adult male Sprague Dawley rats to acute hypocapnic hypoxia. Rats are treated with compounds and exposed to acute hypocapnic hypoxia (12% O₂ balanced with N₂) under anesthesia. Animals can be additionally instrumented with ECG electrodes, invasive arterial blood pressure catheters, pulse oximeters and esophageal catheters as a surrogate for pleural pressure. Changes in pleural pressure measured by esophageal catheter are used to approximate breathing rate. Blood pressure, heart rate, and breathing rate were monitored during baseline conditions (21% O₂ balanced with N₂) and during exposure to hypoxia. Response to hypoxia in vehicle treated rats is shown in FIG. 1.

This model system is used to evaluate the tonic activity of the peripheral chemoreflex, as well as the sensitivity of the peripheral chemoreflex to isocapnic hypoxia, hypocapnic hypoxia, hypercapnic hypoxia, normoxic hypercapnia, or hyperoxia.

### In Vivo Assay for Ventilatory Response in Conscious Animals by Whole-Body Plethysmography

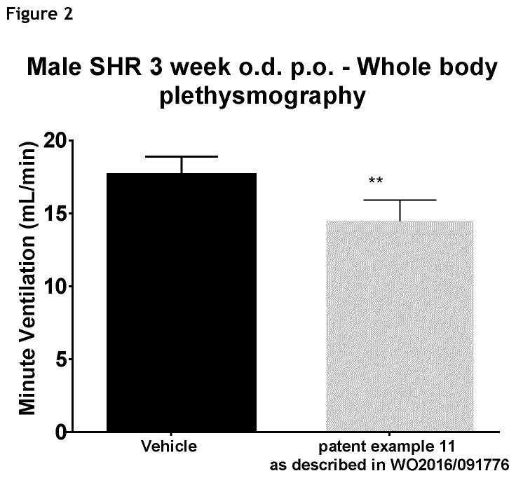

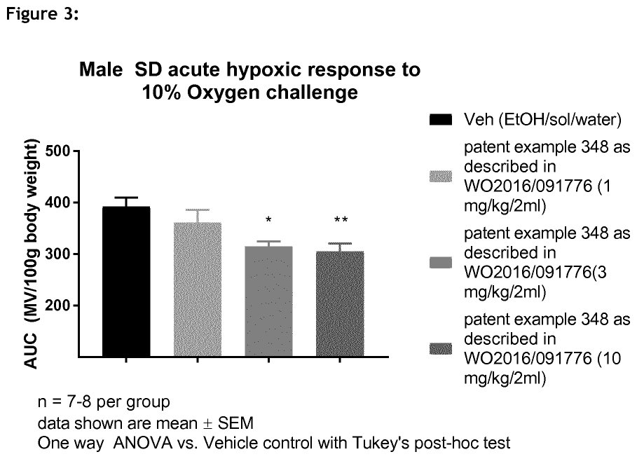

The aim of this study is to test the effect of P2X3 inhibitors on the chemoreflex sensitivity in awake animals. Reduction in ventilation at rest is interpreted as a reduction in chemoreflex sensitivity. In addition, reduction in ventilatory response (as measured by minute ventilation) to an acute hypoxic challenge is interpreted as a reduction in chemoreflex sensitivity. Using this system, we demonstrate reduced minute ventilation in spontaneously hypertensive rats treated for three weeks with P2X3 inhibitor patent example 11 as described in WO2016/091776 (FIG. 2) and after single administration in Sprague dawley rats with P2X3 inbibitor patent example 348 as described in WO2016/091776 (FIG. 3).

Whole body plethysmography involves measuring the flow from breathing of animals in a closed chamber. Animals are placed in small chambers with controlled atmosphere and their breathing is measured in response to changing air composition. In this case, a small animal whole body plethysmography system purchased from Data Sciences International was used. With this method the breathing rate, inhalation and exhalation volume can be measured simultaneously. The minute ventilation, calculated as the product of tidal volume and breathing rate, is a measurement of the respiratory drive of the subject. Whole body plethysmography can be combined with exposure to defined mixtures of gas to measure the response of the subject to specific atmospheric conditions. For example, “normoxia” represents a normal atmospheric oxygen concentration from sea level of 21%. “Hypoxia” represents oxygen concentrations under 21%. Generally for testing physiological responses to hypoxia concentrations of 10-12% oxygen are used. These conditions can be achieved either with an oxygen scrubber to remove oxygen from room air, or by controlling the flow of nitrogen and oxygen to specific concentrations.

### In Vivo Assay for Blood Pressure Monitoring in Conscious Animals by Radiotelemetry

The aim of this study is to evaluate the effect of P2X3 inhibitors on the systemic blood pressure of experimental animals. For P2X3 inhibitors are reported to lower the blood pressure in animal models of hypertension (spontaneously hypertensive rats, SHR). Therefore, a reduction in blood pressure in SHR but not in healthy normotensive animals is interpreted as an on-target antihypertensive effect.

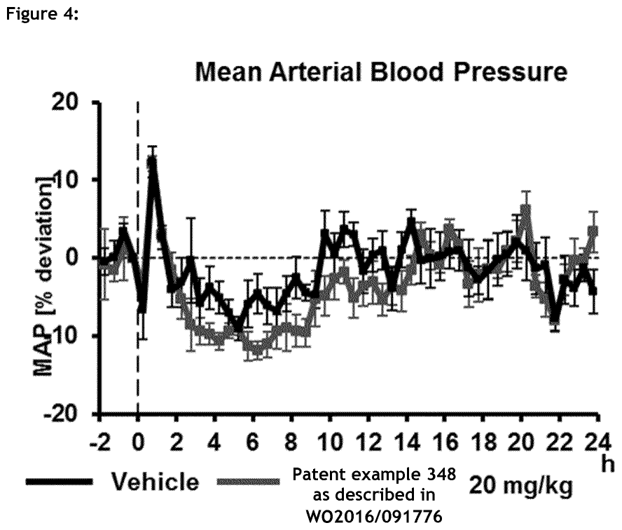

Female SHR or healthy control Wistar Kyoto rats are instrumented with radiotelemetric devices measuring systemic blood pressure by pressure catheters permanently fixed in the abdominal aorta of the animals. Blood pressure is monitored continuously for 24 hours prior to substance application and 48 hours post substance application. Animals are treated p.o. with substance or placebo. Reference to In these experiments we observed a slight reduction in blood pressure in spontaneously hypertensive rats, indicating a mild on-target antihypertensive effect (FIG. 4).

1: A method for treatment or prophylaxis of a disease or a disorder associated with nerve fiber sensitization, and/or other pathological conditions associated with autonomic imbalance caused by increased chemoreceptor sensitivity in a subject in need thereof, in particular for the treatment of breathing disorders, Cheyne Stokes respiration, central and obstructive sleep apnea, cardiovascular disease, hypertension, resistant hypertension, and heart failure, which are related to increased activity of P2X3 receptors, comprising administering to the subject an effective amount of a compound of formula (I): 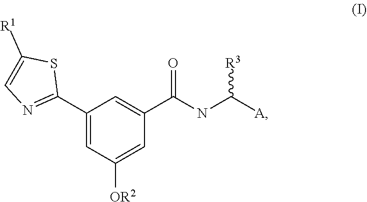
wherein
R¹ is a halogen atom, C₁-C₄-alkyl or C₃-C₆-cycloalkyl,
wherein said C₁-C₄-alkyl is optionally substituted with 1-5 halogen atoms which are the same or different;
R² is —C₂-C₆-alkyl-OR⁴, —(CH₂)_{q}—(C₃-C₇-cycloalkyl), —(CH₂)_{q}-(6- to 12-membered heterobicycloalkyl), —(CH₂)_{q}-(4- to 7-membered heterocycloalkyl), —(CH₂)_{q}-(5- to 10-membered heteroaryl) or —C₂-C₆-alkynyl;
wherein said —(CH₂)_{q}—(C₃-C₇-cycloalkyl), —(CH₂)_{q}-(6- to 12-membered heterobicycloalkyl) and —(CH₂)_{q}-(4- to 7-membered heterocycloalkyl) are optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R, COOR⁵, and oxo (═O),
wherein any ring nitrogen atom, if present in said —(CH₂)_{q}-(6- to 12-membered heterobicycloalkyl) and —(CH₂)_{q}-(4- to 7-membered heterocycloalkyl), is independently substituted with R^{c}; and
wherein said —(CH₂)_{q}-(5- to 10-membered heteroaryl) is optionally substituted with one or more substituents which are the same or different, and selected from the group consisting of C₁-C₄-alkyl optionally substituted with 1-5 halogen atoms which are the same or different, a halogen atom, —NR^{a}R^{b} and —COOR⁵;
R³ is hydrogen or C₁-C₄-alkyl optionally substituted with 1-5 halogen atoms which are the same or different;
R⁴ and R⁵ are independently hydrogen or C₁-C₄-alkyl;
R^{a} and R^{b} are independently hydrogen or C₁-C₄-alkyl;
R^{c} is hydrogen, C₁-C₄-alkyl optionally substituted with 1-5 halogen atoms which are the same or different, —C(O)O—C₁-C₄-alkyl, or —C(O)—C₁-C₄-alkyl;
A is 5- to 10-membered heteroaryl which is optionally substituted with one or more substituents, which are the same or different, and selected from the group consisting of a halogen atom, C₁-C₃-alkyl, and C₁-C₃-alkoxy,
wherein said C₁-C₃-alkyl and C₁-C₃-alkoxy are optionally substituted with 1-5 halogen atoms which are the same or different; and
q is an integer of 0, 1, or 2,
or an isomer, enantiomer, diastereomer, racemate, hydrate, solvate, or salt thereof, or a mixture of any of the foregoing.

2: The method according to claim 1, wherein the compound is of formula (Ia): 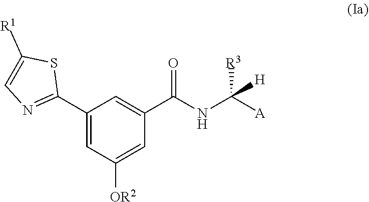
wherein A, R¹, R² and R³ are as defined in claim 1
or an isomer, enantiomer, diastereomer, racemate, hydrate, solvate, or salt thereof, or a mixture of any of the foregoing.

3: The method according to claim 1, wherein the compound is
3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide; or
3-(5-methyl-1,3-thiazol-2-yl)-5-[(3S)-tetrahydrofuran-3-yloxy]-N-{(1R)-1-[2-(trifluoromethyl)pyrimidin-5-yl]ethyl}benzamide;
or a hydrate, solvate, or salt thereof, or a mixture of any of the foregoing.

4: The method according to claim 3, wherein the compound is 3-(5-methyl-1,3-thiazol-2-yl)-5-[(3R)-tetrahydrofuran-3-yloxy]-N-{(1R)-1-[2-(trifluoromethyl)pyrimidin-5-yl]ethyl}benzamide or a hydrate, solvate, or salt thereof, or a mixture of any of the foregoing.

5: The method according to claim 1 wherein the compound is
3-{[(2S)-4-methylmorpholin-2-yl]methoxy}-5-(5-methyl-1,3-thiazol-2-yl)-N-{(1R)-1-[2-(trifluoromethyl)pyrimidin-5-yl]ethyl}benzamide; or
3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide;
or a hydrate, solvate, or salt thereof, or a mixture of any of the foregoing.

6: The method according to claim 5, wherein the compound is 3-{[(2R)-4-methylmorpholin-2-yl]methoxy}-5-(5-methyl-1,3-thiazol-2-yl)-N-{(1R)-1-[2-(trifluoromethyl)-pyrimidin-5-yl]ethyl}benzamide or a hydrate, solvate, or salt thereof, or a mixture of any of the foregoing.

7: A method for treatment or prophylaxis of a disease or a disorder associated with nerve fiber sensitization, and/or other pathological conditions associated with autonomic imbalance caused by increased chemoreceptor sensitivity in a subject in need thereof, in particular for the treatment of breathing disorders, Cheyne Stokes respiration, central and obstructive sleep apnea, cardiovascular disease, hypertension, resistant hypertension, and heart failure, which are related to increased activity of P2X3 receptors, comprising administering to the subject an effective amount of a pharmaceutical composition comprising a compound of formula (I): 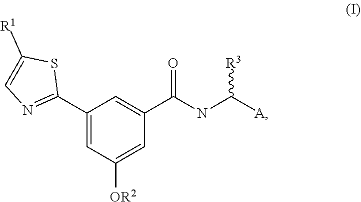
wherein
R¹ is a halogen atom, C₁-C₄-alkyl or C₃-C₆-cycloalkyl,
wherein said C₁-C₄-alkyl is optionally substituted with 1-5 halogen atoms which are the same or different;
R² is —C₂-C₆-alkyl-OR⁴, —(CH₂)_{q}—(C₃-C₇-cycloalkyl), —(CH₂)_{q}-(6- to 12-membered heterobicycloalkyl), —(CH₂)_{q}-(4- to 7-membered heterocycloalkyl), —(CH₂)_{q}-(5- to 10-membered heteroaryl) or —C₂-C₆-alkynyl,
wherein said —(CH₂)_{q}—(C₃-C₇-cycloalkyl), —(CH₂)_{q}-(6- to 12-membered heterobicycloalkyl) and —(CH₂)_{q}-(4- to 7-membered heterocycloalkyl) are optionally substituted with one or more substituents which are the same or different, at any ring carbon atom and selected from the group consisting of C₁-C₄ alkyl, optionally substituted with 1-5 halogen atoms which are the same or different, a halogen atom, —NR^{a}R^{b}, COOR⁵, and oxo (═O),
wherein any ring nitrogen atom, if present in said —(CH₂)_{q}-(6- to 12-membered heterobicycloalkyl) and —(CH₂)_{q}-(4- to 7-membered heterocycloalkyl), is independently substituted with R^{c}; and
wherein said —(CH₂)_{q}-(5- to 10-membered heteroaryl) is optionally substituted with one or more substituents which are the same or different, and selected from the group consisting of C₁-C₄-alkyl optionally substituted with 1-5 halogen atoms which are the same or different, a halogen atom, —NR^{a}R^{b} and —COOR⁵;
R³ is hydrogen or C₁-C₄-alkyl optionally substituted with 1-5 halogen atoms which are the same or different;
R⁴ and R⁵ are independently hydrogen or C₁-C₄-alkyl;
R^{a} and R^{b} are independently hydrogen or C₁-C₄-alkyl;
R^{c} is hydrogen, C₁-C₄-alkyl optionally substituted with 1-5 halogen atoms which are the same or different, —C(O)O—C₁-C₄-alkyl, or —C(O)—C₁-C₄-alkyl;
A is 5- to 10-membered heteroaryl which is optionally substituted with one or more substituents, which are the same or different, and selected from the group consisting of a halogen atom, C₁-C₃-alkyl, and C₁-C₃-alkoxy,
wherein said C₁-C₃-alkyl and C₁-C₃-alkoxy are optionally substituted with 1-5 halogen atoms which are the same or different; and
q is an integer of 0, 1, or 2,
or an isomer, enantiomer, diastereomer, racemate, hydrate, solvate, or salt thereof, or a mixture of any of the foregoing, and a pharmaceutically acceptable diluent or carrier.

8: The method according to claim 1, wherein the method is a method of long-term treatment or prophylaxis.

9: The method according to claim 1, wherein the method is a method of oral treatment or prophylaxis.

10: The method according to claim 7, wherein the method is a method of long-term treatment or prophylaxis.

11: The method according to claim 7, wherein the method is a method of oral treatment or prophylaxis.
